(19) United States
(12) Patent Application Publication
(10) Pub. No.: US 2015/0132301 A1
(43) Pub. Date: May 14, 2015

(54) Combination Therapy for Treatment of Cancer

(71) Applicant: OncoMed Pharmaceuticals, Inc., Redwood City, CA (US)

(72) Inventors: Timothy C. Hoey, Hillsborough, CA (US); Christopher Lamond Murriel, San Francisco, CA (US)

(21) Appl. No.: 14/362,991

(22) PCT Filed: Dec. 7, 2012
(86) PCT No.: PCT/US2012/068351

§ 371 Date: Jun. 5, 2014

Related U.S. Application Data
(60) Provisional application No. 61/568,844, filed on Dec. 9, 2011.
(60) Provisional application No. 61/698,030, filed on Sep. 7, 2012.

(51) Int. Cl. A61K 39/395; A61K 38/17; A61K 31/18

(52) U.S. Cl. CPC A61K 39/3955; A61K 31/18; A61K 38/177
USPC 424/134.1; 424/139.1; 424/133.1; 424/136.1

(57) ABSTRACT

The present invention provides methods comprising combination therapy for treating cancer. In particular, the present invention provides Wnt pathway inhibitors in combination with MAPK pathway inhibitors for the treatment of cancer and other diseases. In some embodiments, the MAPK pathway signaling activation is due to a mutation in a MAPK pathway component. In some embodiments, the MAPK pathway signaling component is Ras, Raf, MEK, or ERK.

### CROSS-REFERENCE TO RELATED APPLICATIONS

This application claims priority benefit of U.S. Provisional Application No. 61/568,844, filed Dec. 9, 2011 and U.S. Provisional Application No. 61/698,030, filed Sep. 7, 2012, each of which is hereby incorporated by reference herein in its entirety.

### FIELD OF THE INVENTION

The present invention provides methods comprising combination therapy for treating cancer. In particular, the present invention provides Wnt pathway inhibitors in combination with MAPK pathway inhibitors for the treatment of cancer and other diseases.

### BACKGROUND OF THE INVENTION

Cancer is one of the leading causes of death in the developed world, with over one million people diagnosed with cancer and 500,000 deaths per year in the United States alone. Overall it is estimated that more than 1 in 3 people will develop some form of cancer during their lifetime. There are more than 200 different types of cancer, four of which—breast, lung, colorectal, and prostate—account for over half of all new cases (Siegel et al., 2011, CA: Cancer J. Clin., 61:212-236). Skin cancer is the most common of all cancers and melanoma is the most serious and aggressive type of skin cancer. Melanoma accounts for less than 5% of skin cancer cases, yet it is responsible for a large majority of the deaths associated with skin cancer. The survival rate is fairly high for individuals who are diagnosed with early stage melanoma and receive appropriate treatment. However, metastatic melanoma is highly aggressive and remains one of the most difficult cancers to treat. Individuals with this advanced form have overall survival rates of less than 10% and median survival times of only six to nine months.

Signaling pathways normally connect extracellular signals to the nucleus leading to expression of genes that directly or indirectly control cell growth, differentiation, survival, and death. In a wide variety of cancers, signaling pathways are dysregulated and may be linked to tumor initiation and/or progression. Signaling pathways implicated in human oncogenesis include, but are not limited to, the Wnt pathway, the Ras-Raf-MEK-ERK or MAPK pathway, the PI3K-AKT pathway, the CDKN2A/CDK4 pathway, the Bcl-2/TP53 pathway, and the Notch pathway.

The Wnt signaling pathway has been identified as a potential target for cancer therapy. The Wnt signaling pathway is one of several critical regulators of embryonic pattern formation, post-embryonic tissue maintenance, and stem cell biology. More specifically, Wnt signaling plays an important role in the generation of cell polarity and cell fate specification including self-renewal by stem cell populations. Unregulated activation of the Wnt pathway is associated with numerous human cancers where it is believed the activation can alter the developmental fate of cells. The activation of the Wnt pathway may maintain tumor cells in an undifferentiated state and/or lead to uncontrolled proliferation. Thus carcinogenesis can proceed by overtaking homeostatic mechanisms which control normal development and tissue repair (reviewed in Reya & Clevers, 2005, Nature, 434:843-50; Beachy et al., 2004, Nature, 432:324-31).

The Wnt signaling pathway was first elucidated in the Drosophila developmental mutant wingless (wg) and from the murine proto-oncogene int-1, now Wnt1 (Nusse & Varmus, 1982, Cell, 31:99-109; Van Ooyen & Nusse, 1984, Cell, 39:233-40; Cabrera et al., 1987, Cell, 50:659-63; Rijsewijk et al., 1987, Cell, 50:649-57), Wnt genes encode secreted lipid-modified glycoproteins of which 19 have been identified in mammals. These secreted ligands activate a receptor complex consisting of a Frizzled (FZD) receptor family member and low-density lipoprotein (LDL) receptor-related protein 5 or 6 (LRP5/6). The FZD receptors are seven transmembrane domain proteins of the G-protein coupled receptor (GPCR) superfamily and contain a large extracellular N-terminal ligand binding domain with 10 conserved cysteines, known as a cysteine-rich domain (CRD) or Fri domain. There are ten human FZD receptors, FZD1, FZD2, FZD3, FZD4, FZD5, FZD6, FZD7, FZD8, FZD9, and FZD10. Different FZD CRDs have different binding affinities for specific Wnt proteins (Wu & Nusse, 2002, J. Biol. Chem., 277:41762-9), and FZD receptors have been grouped into those that activate the canonical β-catenin pathway and those that activate non-canonical pathways (Miller et al., 1999, Oncogene, 18:7860-72).

A role for Wnt signaling in cancer was first uncovered with the identification of Wnt1 (originally int1) as an oncogene in mammary tumors transformed by the nearby insertion of a murine virus (Nusse & Varmus, 1982, Cell, 31:99-109). Additional evidence for the role of Wnt signaling in breast cancer has since accumulated. For instance, transgenic over-expression of β-catenin in the mammary glands results in hyperplasias and adenocarcinomas (Imbert et al., 2001, J. Cell Biol., 153:555-68; Michaelson & Leder, 2001, Oncogene, 20:5093-9) whereas loss of Wnt signaling disrupts normal mammary gland development (Tepera et al., 2003, J. Cell Sci., 116:1137-49; Hatsell et al., 2003, J. Mammary Gland Biol. Neoplasia, 8:145-58). In human breast cancer, β-catenin accumulation implicates activated Wnt signaling in over 50% of carcinomas, and though specific mutations have not been identified, up-regulation of Frizzled receptor expression has been observed (Brennan & Brown, 2004, J. Mammary Gland Biol. Neoplasia, 9:119-31; Malovanovic et al., 2004, Int. J. Oncol., 25:1337-42).

Activation of the Wnt pathway is also associated with colorectal cancer. Approximately 5-10% of all colorectal cancers are hereditary with one of the main forms being familial adenomatous polyposis (FAP), an autosomal dominant disease in which about 80% of affected individuals contain a germline mutation in the adenomatous polyposis coli (APC) gene. Mutations have also been identified in other Wnt pathway components including Axin and β-catenin. Individual adenomas are clonal outgrowths of epithelial cells containing a second inactivated allele, and the large number of FAP adenomas inevitably results in the development of adenocarcinomas through additional mutations in oncogenes and/or tumor suppressor genes. Furthermore, activation of the Wnt signaling pathway, including loss-of-function mutations in APC and stabilizing mutations in β-catenin, can induce hyperplastic development and tumor growth in mouse models (Oshima et al., 1997, Cancer Res., 57:1644-9; Harada et al., 1999, EMBO J., 18:5931-42).

Similar to breast cancer and colon cancer, melanoma often has constitutive activation of the Wnt pathway, as indicated by the nuclear accumulation of β-catenin. Activation of the Wnt/β-catenin pathway in some melanoma tumors and cell lines is due to modifications in pathway components, such as APC, ICAT, LEF1 and β-catenin (see e.g., Larue et al., 2006, Frontiers Biosci., 11:733-742). However, there are conflicting reports in the literature as to the exact role of Wnt/β-catenin signaling in melanoma. For example, one study found that elevated levels of nuclear β-catenin correlated with improved survival from melanoma, and that activated Wnt/β-catenin signaling was associated with decreased cell proliferation (Chien et al., 2009, PNAS, 106:1193-1198).

The MAPK (mitogen-activated protein kinase) pathway has been shown to play a key role in a number of normal physiological processes such as cellular metabolism, cell cycle progression, cell death, and neurological function. The MAPK pathway is constitutively activated in a significant proportion of human tumors often through gain-of-function mutations in Ras or Raf gene family members. Mutations in the MAPK pathway have been shown to be very important in melanoma development in that up to 90% of melanomas and benign melanocytic neoplasms carry activating mutations in B-Raf, K-Ras, or N-Ras. In addition, it has been reported that 30-70% of malignant melanomas contain B-Raf mutations and that a valine to glutamate change at position 600 accounts for approximately 80% of the mutations. (Davies et al., 2002, Nature, 417:949-954). Mutations in Ras genes are also found in lung cancers, such as non-small cell lung cancer (NSCLC) and studies have shown that patients with mutant K-Ras lung tumors do not respond to EGFR inhibitors (Riely et al., 2009, Proc. Am. Thorac. Soc.; 6:201-205).

The focus of cancer drug research is shifting toward targeted therapies aimed at genes and pathways involved in human cancer. For example, numerous efforts to develop therapeutic agents that specifically target the mutated B-Raf kinase are currently underway for melanoma treatment. However, the development of resistance to the B-Raf inhibitors has proven to be a major challenge. Furthermore, these agents have little or no effect in patients whose tumors have a wild-type B-Raf. In fact, patients without the V600E B-Raf mutation have been excluded from on-going clinical trials.

Thus, there is a need for new agents targeting signaling pathways and new combinations of agents that target multiple pathways that could provide therapeutic benefit for cancer patients.

### BRIEF SUMMARY OF THE INVENTION

The present invention provides methods of treating diseases comprising administering a Wnt pathway inhibitor in combination with a MAPK pathway inhibitor to a subject in need thereof. Combination therapy with at least two therapeutic agents often uses agents that work by different mechanisms of action, and/or target different pathways and may result in additive or synergetic effects. Importantly, combination therapy may allow for a lower dose of each agent than used in monotherapy, thereby reducing toxic side effects and/or increasing the therapeutic index of the agent(s). Also, combination therapy may decrease the likelihood that resistance to an agent will develop.

The invention provides Wnt pathway inhibitors, including but not limited to, antibodies and other polypeptides that bind at least one Wnt protein(s), antibodies and other polypeptides that bind at least one FZD protein(s), and Wnt-binding soluble receptors. The invention also provides Wnt pathway inhibitors that are small molecules. The invention provides MAPK pathway inhibitors, including but not limited to, small molecules that are MEK inhibitors, Raf inhibitors, Ras inhibitors, and ERK inhibitors. Compositions (e.g., pharmaceutical compositions) comprising the Wnt pathway inhibitors and/or the MAPK pathway inhibitors are also provided.

Thus in one aspect, the invention provides methods of inhibiting tumor growth. In some embodiments, the method comprises contacting tumor cells with an effective amount of a Wnt pathway inhibitor in combination with an effective amount of a MAPK pathway inhibitor. The method may be in vivo or in vitro. In certain embodiments, the tumor is in a subject, and contacting tumor cells with the Wnt pathway inhibitor and the MAPK pathway inhibitor comprises administering a therapeutically effective amount of each of the inhibitors to the subject.

In another aspect, the invention provides methods of treating cancer in a subject comprising administering to the subject a therapeutically effective amount of a Wnt pathway inhibitor in combination with a therapeutically effective amount of a MAPK pathway inhibitor.

In another aspect, the invention provides methods of treating a disease associated with Wnt pathway activation, comprising administering a therapeutically effective amount of a Wnt pathway inhibitor and a therapeutically effective amount of a MAPK pathway inhibitor to a subject.

In another aspect, the invention provides methods of treating a disease associated with MAPK pathway activation, comprising administering a therapeutically effective amount of a Wnt pathway inhibitor in combination with a therapeutically effective amount of a MAPK pathway inhibitor to a subject. In some embodiments, the MAPK pathway signaling activation is due to a mutation in a MAPK pathway component. In some embodiments, the MAPK pathway component is Ras, Raf, MEK, or ERK.

In another aspect, the invention provides methods of treating a human subject, comprising (a) determining if the subject has a cancer or tumor comprising a mutation in the MAPK pathway, and (b) administering to the subject a therapeutically effective amount of a Wnt pathway inhibitor in combination with a therapeutically effective amount of a MAPK pathway inhibitor. In some embodiments, the MAPK pathway comprises a wild-type B-Raf. In some embodiments, the MAPK pathway comprises a B-Raf mutation. In some embodiments, the MAPK pathway comprises a wild-type Ras. In some embodiments, the MAPK pathway comprises a Ras mutation. In some embodiments, the MAPK pathway comprises a wild-type N-Ras. In some embodiments, the MAPK pathway comprises an N-Ras mutation. In some embodiments, the MAPK pathway comprises a wild-type K-Ras. In some embodiments, the MAPK pathway comprises a K-Ras mutation.

In another aspect, the invention provides methods of treating a human subject, comprising (a) selecting a subject for treatment based on, at least in part, the subject having a tumor or cancer that comprises a mutation in the MAPK pathway, and (b) administering to the subject a therapeutically effective amount of a Wnt pathway inhibitor in combination with a therapeutically effective amount of a MAPK pathway inhibitor. In some embodiments, the MAPK pathway comprises a wild-type B-Raf. In some embodiments, the MAPK pathway comprises a B-Raf mutation. In some embodiments, the MAPK pathway comprises a wild-type Ras. In some embodiments, the MAPK pathway comprises a Ras mutation. In some embodiments, the MAPK pathway comprises a wild-type N-Ras. In some embodiments, the MAPK pathway comprises an N-Ras mutation. In some embodiments, the MAPK pathway comprises a wild-type K-Ras. In some embodiments, the MAPK pathway comprises a K-Ras mutation.

In another aspect, the invention provides methods of treating a human subject who has a tumor or cancer, wherein the tumor or cancer is substantially non-responsive to at least one B-Raf inhibitor, and wherein the method comprises administering to the subject a therapeutically effective amount of a Wnt pathway inhibitor in combination with a therapeutically effective amount of a MAPK pathway inhibitor.

In another aspect, the invention provides methods of treating a human subject, comprising (a) selecting a subject for treatment based on, at least in part, the subject having a tumor or cancer which is substantially non-responsive to at least one B-Raf inhibitor, and (b) administering to the subject a therapeutically effective amount of a Wnt pathway inhibitor in combination with a therapeutically effective amount of a MAPK pathway inhibitor. In some embodiments, the cancer or tumor that is substantially non-responsive to at least one B-Raf inhibitor comprises a wild-type B-Raf. In some embodiments, the cancer or tumor that is substantially non-responsive to at least one B-Raf inhibitor has been previously treated with a B-Raf inhibitor. In some embodiments, the subject has been previously treated with a B-Raf inhibitor.

In another aspect, the invention provides methods of treating a human subject who has a tumor or cancer comprising a B-Raf mutation, comprising administering to the subject a therapeutically effective amount of a Wnt pathway inhibitor in combination with a therapeutically effective amount of a MAPK pathway inhibitor.

In another aspect, the invention provides methods of treating a human subject who has a tumor or cancer comprising a N-Ras mutation, comprising administering to the subject a therapeutically effective amount of a Wnt pathway inhibitor in combination with a therapeutically effective amount of a MAPK pathway inhibitor.

In another aspect, the invention provides methods of treating a human subject who has a tumor or cancer comprising a K-Ras mutation, comprising administering to the subject a therapeutically effective amount of a Wnt pathway inhibitor in combination with a therapeutically effective amount of a MAPK pathway inhibitor.

In certain embodiments of each of the aforementioned aspects, as well as other aspects and embodiments described elsewhere herein, a mutation (or lack thereof) in the MAPK pathway is detected in a sample by methods known to those skilled in the art, such as PCR-based assays or direct nucleotide sequencing. In some embodiments, the mutation is a B-Raf mutation. In some embodiments, the mutation is an N-Ras mutation. In some embodiments, the mutation is a K-Ras mutation. In some embodiments, the sample is a fresh sample, a frozen sample, or a formalin-fixed paraffin-embedded sample.

In certain embodiments of each of the aforementioned aspects, as well as other aspects and embodiments described elsewhere herein, the Wnt pathway inhibitor is an antibody that specifically binds at least one human Wnt protein. In some embodiments, the Wnt pathway inhibitor is an antibody that specifically binds at least one human FZD protein.

In some embodiments, the Wnt pathway inhibitor is an antibody comprising a heavy chain CDR1 comprising GFTFSHYTLS (SEQ ID NO:5), a heavy chain CDR2 comprising VISGDGSYTYYADSVKG (SEQ ID NO:6), and a heavy chain CDR3 comprising NFIKYVFAN (SEQ ID NO:7), and/or a light chain CDR1 comprising SGDNIGSFYVH (SEQ ID NO:8), a light chain CDR2 comprising DKSNRPSG (SEQ ID NO:9), and a light chain CDR3 comprising QSYANTLSL (SEQ ID NO: 10).

In certain embodiments of each of the aforementioned aspects, as well as other aspects and embodiments described elsewhere herein, the Wnt pathway inhibitor is an antibody comprising (a) a heavy chain variable region having at least about 90%, at least about 95%, or 100% sequence identity to SEQ ID NO:3; and/or (b) a light chain variable region having at least about 90%, at least about 95%, or 100% sequence identity to SEQ ID NO:4. In some embodiments, the Wnt pathway inhibitor is antibody 18R5.

In certain embodiments of each of the aforementioned aspects, as well as other aspects and embodiments described elsewhere herein, the Wnt pathway inhibitor is a recombinant antibody. In some embodiments, the antibody is a monoclonal antibody, a chimeric antibody, a humanized antibody, or a human antibody. In some embodiments, the antibody is an antibody fragment comprising an antigen-binding site. In certain embodiments, the antibody or antibody fragment is monovalent, monospecific, bivalent, bispecific, or multispecific. In some embodiments, the antibody is an IgG1 antibody or an IgG2 antibody. In certain embodiments, the antibody is isolated. In other embodiments, the antibody is substantially pure.

In certain embodiments of each of the aforementioned aspects, as well as other aspects and embodiments described elsewhere herein, the Wnt pathway inhibitor is a soluble receptor. In some embodiments, the soluble receptor comprises a Fri domain of a human FZD protein. In some embodiments, the Fri domain of the human FZD protein comprises a sequence selected from the group consisting of: SEQ ID NO: 11, SEQ ID NO:12, SEQ ID NO: 13, SEQ ID NO:14, SEQ ID NO: 15, SEQ ID NO:16, SEQ ID NO:17, SEQ ID NO:18, SEQ ID NO:19, SEQ ID NO:20, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, and SEQ ID NO:58. In some embodiments, the Fri domain of the human FZD protein is directly linked to a non-FZD polypeptide. In some embodiments, the Fri domain of the human FZD protein is connected to a non-FZD polypeptide by a linker. In some embodiments, the non-FZD polypeptide comprises a human Fc region. In some embodiments, the non-FZD polypeptide consists essentially of SEQ ID NO:21, SEQ ID NO:22, SEQ ID NO:23, SEQ ID NO:24, or SEQ ID NO:59. In some embodiments, the Wnt pathway inhibitor comprises (a) a first polypeptide consisting essentially of SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, or SEQ ID NO:58; and (b) a second polypeptide consisting essentially of SEQ ID NO:21, SEQ ID NO:22, SEQ ID NO:23, SEQ ID NO:24, or SEQ ID NO:59, wherein the first polypeptide is directly linked to the second polypeptide. In some embodiments, the Wnt pathway inhibitor comprises (a) a first polypeptide consisting essentially of SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, or SEQ ID NO:58; and (b) a second polypeptide consisting essentially of SEQ ID NO:21, SEQ ID NO:22, SEQ ID NO:23, SEQ ID NO:24, or SEQ ID NO:59, wherein the first polypeptide is connected to the second polypeptide by a linker. In some embodiments, the Wnt pathway inhibitor comprises SEQ ID NO:25, SEQ ID NO:26, or SEQ ID NO:27. In some embodiments, the Wnt pathway inhibitor comprises SEQ ID NO:27.

In certain embodiments of each of the aforementioned aspects, as well as other aspects and embodiments described elsewhere herein, the MAPK pathway inhibitor is selected from a group consisting of a MEK inhibitor, a Ras inhibitor, a Raf inhibitor, and a ERK inhibitor. In some embodiments, the MAPK pathway inhibitor is a MEK inhibitor. In some embodiments, the MEK inhibitor is selected from the group consisting of: BAY 86-9766 (RDEA119), PD0325901, CI-1040, PD98059, PD318088, GSK1120212 (JTP-74057), AZD8330 (ARRY-424704), AZD6244 (ARRY-142886), ARRY-162, ARRY-300, AS703026, U0126, CH4987655, and TAK-733. In some embodiments, the MEK inhibitor is BAY 86-9766. In some embodiments, the MAPK pathway inhibitor is a Raf inhibitor. In some embodiments, the Raf inhibitor is selected from the group consisting of GDC-0879, PLX-4720, PLX-4032 (vemurafenib), RAF265, BAY 73-4506, BAY 43-9006 (sorafenib), SB590885, XL281 (BMS-908662), GDC-0879, and GSK 2118436436. In some embodiments, the MAPK pathway inhibitor is a Ras inhibitor. In some embodiments, the Ras inhibitor is farnesylthiosalicylic acid (FTS). In some embodiments, the MAPK pathway inhibitor is an ERK inhibitor.

In some embodiments, the invention provides a method of inhibiting growth of a melanoma tumor in a subject, comprising administering to the subject a therapeutically effective amount of an anti-FZD antibody in combination with a MEK inhibitor.

In some embodiments, the invention provides a method of inhibiting growth of a melanoma tumor in a subject, comprising administering to the subject a therapeutically effective amount of anti-FZD antibody 18R5 in combination with a MEK inhibitor.

In some embodiments, the invention provides a method of inhibiting growth of a melanoma tumor in a subject, comprising administering to the subject a therapeutically effective amount of anti-FZD antibody 18R5 in combination with MEK inhibitor BAY 86-9766.

In some embodiments, the invention provides a method of inhibiting growth of a lung tumor in a subject, comprising administering to the subject a therapeutically effective amount of an anti-FZD antibody in combination with a MEK inhibitor.

In some embodiments, the invention provides a method of inhibiting growth of a lung tumor in a subject, comprising administering to the subject a therapeutically effective amount of anti-FZD antibody 18R5 in combination with a MEK inhibitor.

In some embodiments, the invention provides a method of inhibiting growth of a lung tumor in a subject, comprising administering to the subject a therapeutically effective amount of anti-FZD antibody 18R5 in combination with MEK inhibitor BAY 86-9766.

In certain embodiments of each of the aforementioned aspects, as well as other aspects and embodiments described elsewhere herein, the methods further comprise administering at least one additional therapeutic agent appropriate for effecting combination therapy. In some embodiments, the additional therapeutic agent is a chemotherapeutic agent. In some embodiments, the additional therapeutic agent is an antibody.

Also provided are pharmaceutical compositions which comprise a Wnt pathway inhibitor and/or a MAPK pathway inhibitor described herein and a pharmaceutically acceptable vehicle (or carrier).

Where aspects or embodiments of the invention are described in terms of a Markush group or other grouping alternatives, the present invention encompasses not only the entire group listed as a whole, but also each member of the group individually and all possible subgroups of the main group, and also the main group absent one or more of the group members. The present invention also envisages the explicit exclusion of one or more of any of the group members in the claimed invention.

### BRIEF DESCRIPTION OF THE DRAWINGS/FIGURES

FIG. 1. Inhibition of melanoma tumor growth in vivo by a Wnt pathway inhibitor in combination with a MAPK pathway inhibitor. OMP-M3 (FIG. 1A), OMP-M7 (FIG. 1B), and OMP-M10 (FIG. 1C) melanoma tumor cells were injected subcutaneously into NOD/SCID mice. Mice were treated with control antibody (-▪-), anti-FZD antibody 18R5 (-▴-), MEK inhibitor BAY 86-9766 (-▾-), or a combination of 18R5 and BAY 86-9766 (--). Data is shown as tumor volume (mm³) over days post-treatment. Antibodies were administered intraperitoneally at 20 mg/kg once a week and MEK inhibitor was administered orally at 15 mg/kg daily for 5 days each week.

FIG. 2. Melanoma tumor growth in vivo in the presence of a B-Raf inhibitor. OMP-M8 melanoma tumor cells were injected subcutaneously into NOD/SCID mice. Mice were treated with control vehicle methyl cellulose (-▪-) or B-Raf inhibitor PLX-4720 at 5 mg/kg (-▴-), 15 mg/kg (-▾-), or 45 mg/kg (--). Data is shown as tumor volume (mm³) over days post-treatment. Control vehicle and PLX-4720 were administered orally for 5 days each week.

FIG. 3. Inhibition of melanoma tumor growth and tumorigenicity in vivo by a Wnt pathway inhibitor in combination with a MAPK pathway inhibitor. OMP-M8 melanoma tumor cells were injected subcutaneously into NOD/SCID mice. Mice were treated with control antibody (-▪-), anti-FZD antibody 18R5 (-▴-), MEK inhibitor BAY 86-9766 (-▾-), or a combination of 18R5 and BAY 86-9766 (--). Antibodies were administered intraperitoneally at 20 mg/kg once a week and MEK inhibitor was administered orally at 30 mg/kg daily for 5 days each week. Data is shown as tumor volume (mm³) over days post-treatment (FIG. 3A). The resulting tumors were processed to single cell suspensions, and serially transplanted into mice. 10 or 100 cells from each treatment group were injected subcutaneously into NOD/SCID mice. Tumors were allowed to grow with no treatment. Data is shown as tumor volume (mm³) on day 51 (FIG. 3B).

FIG. 4. Inhibition of lung tumor growth in vivo by a Wnt pathway inhibitor in combination with a MAPK pathway inhibitor. (FIG. 4A) OMP-LU33 lung tumor cells were injected subcutaneously into NOD/SCID mice. Mice were treated with control antibody (-▪-), anti-FZD antibody 18R5 (-▴-), MEK inhibitor BAY 86-9766 (-▾-), or a combination of 18R5 and BAY 86-9766 (--). Antibodies were administered intraperitoneally at 25 mg/kg once a week and MEK inhibitor was administered orally at 50 mg/kg daily for 5 days each week. (FIG. 4B) OMP-LU56 lung tumor cells were injected subcutaneously into NOD/SCID mice. Mice were treated with control antibody (--), anti-FZD antibody 18R5 (-▪-), MEK inhibitor BAY 86-9766 (-▴-), or a combination of 18R5 and BAY 86-9766 (-▾-). Antibodies were administered intraperitoneally at 25 mg/kg every two weeks (Q2W) and MEK inhibitor was administered orally at 30 mg/kg daily for 5 days each week. Data is shown as tumor volume (mm³) over days post-treatment.

FIG. 5. Inhibition of total and active β-catenin levels in vivo by a Wnt pathway inhibitor in combination with a MAPK pathway inhibitor. OMP-M7 and OMP-M10 melanoma tumors were treated with a control antibody, anti-FZD antibody 18R5, MEK inhibitor BAY 86-9766 (MEKi), or a combination of antibody 18R5 and BAY 86-9766. Western blot analyses of protein lysates from treated tumors were performed with antibodies to active β-catenin, total β-catenin, phosphorylated ERK (pERK), total ERK, and actin (OMP-M7, FIG. 5A and OMP-M10, FIG. 5C). Western blot results of active β-catenin and total β-catenin were measured and quantified using NIH Image J software (OMP-M7, FIG. 5B and OMP-M10, FIG. 5D).

FIG. 6. Inhibition of gene expression by a Wnt pathway inhibitor in combination with a MAPK pathway inhibitor. OMP-M3, OMP-M7, and OMP-M10 melanoma tumors were treated with a control antibody, anti-FZD antibody 18R5, MEK inhibitor BAY 86-9766, or a combination of antibody 18R5 and BAY 86-9766. RNA was isolated from the tumors and was analyzed by TaqMan gene expression assay (FIG. 6A). Formalin-fixed, paraffin-embedded tumor sections from OMP-M3, OMP-M7, and OMP-M10 tumors were analyzed by immunohistochemistry (IHC) using an antibody for E-cadherin. After antibody staining, slides were scanned using an Aperio ScanScope instrument and the human cell populations were analyzed for positive staining using Aperio software (FIG. 6B).

### DETAILED DESCRIPTION OF THE INVENTION

The present invention provides methods of inhibiting tumor growth and methods of treating cancer. The methods provided herein comprise administering to a subject a therapeutically effective amount of a Wnt pathway inhibitor in combination with a therapeutically effective amount of a MAPK pathway inhibitor. In some embodiments, the Wnt pathway inhibitor is an antibody. In some embodiments, the Wnt pathway inhibitor is an antibody that specifically binds at least one Wnt protein. In some embodiments, the Wnt pathway inhibitor is an antibody that specifically binds at least one FZD protein. In some embodiments, the Wnt pathway inhibitor is a soluble receptor. In some embodiments, the Wnt pathway inhibitor is a soluble receptor comprising the Fri domain of a FZD protein. In some embodiments, the MAPK pathway inhibitor is a MEK inhibitor, a Ras inhibitor, a Raf inhibitor, or an ERK inhibitor. In certain embodiments, the MAPK pathway inhibitor is a MEK inhibitor. In some embodiments, an antibody that specifically binds at least one FZD protein is administered in combination with a MEK inhibitor. In some embodiments, an antibody that specifically binds at least one Wnt protein is administered in combination with a MEK inhibitor. In some embodiments, a soluble receptor that binds at least one Wnt protein is administered in combination with a MEK inhibitor.

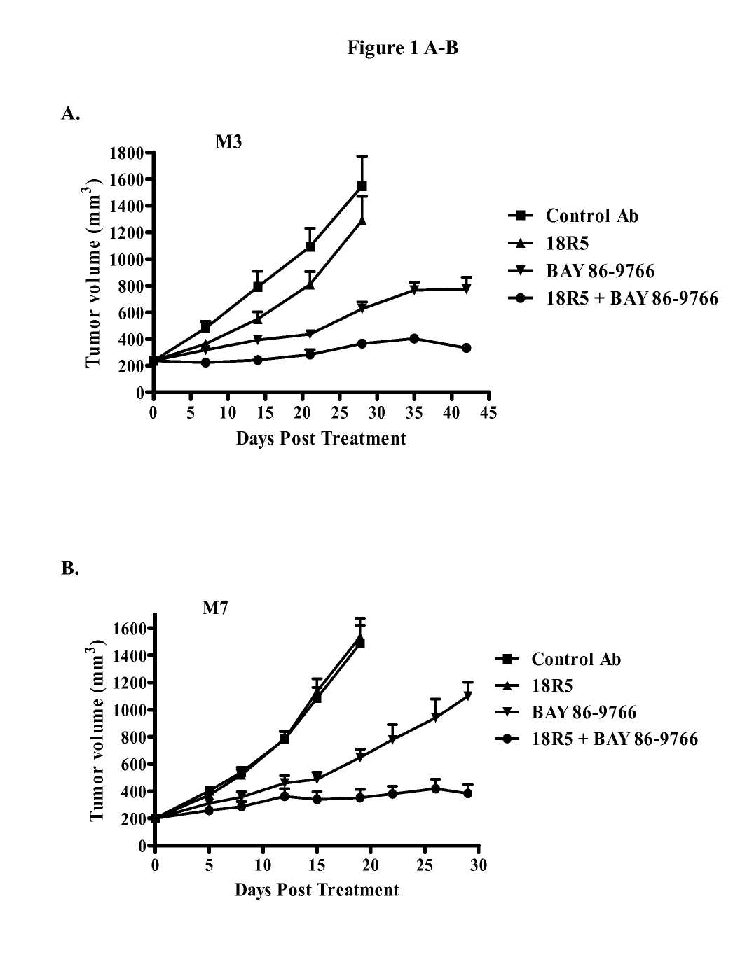

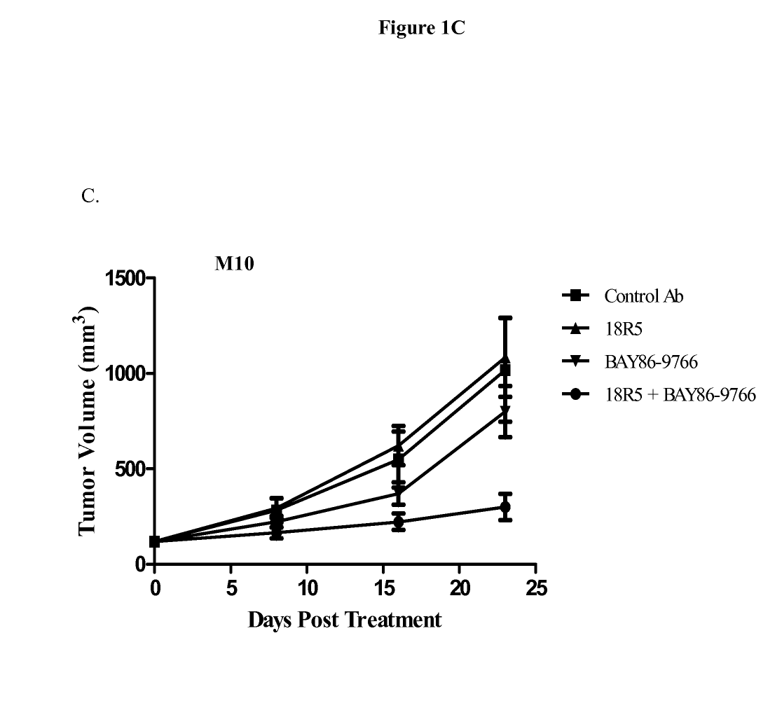

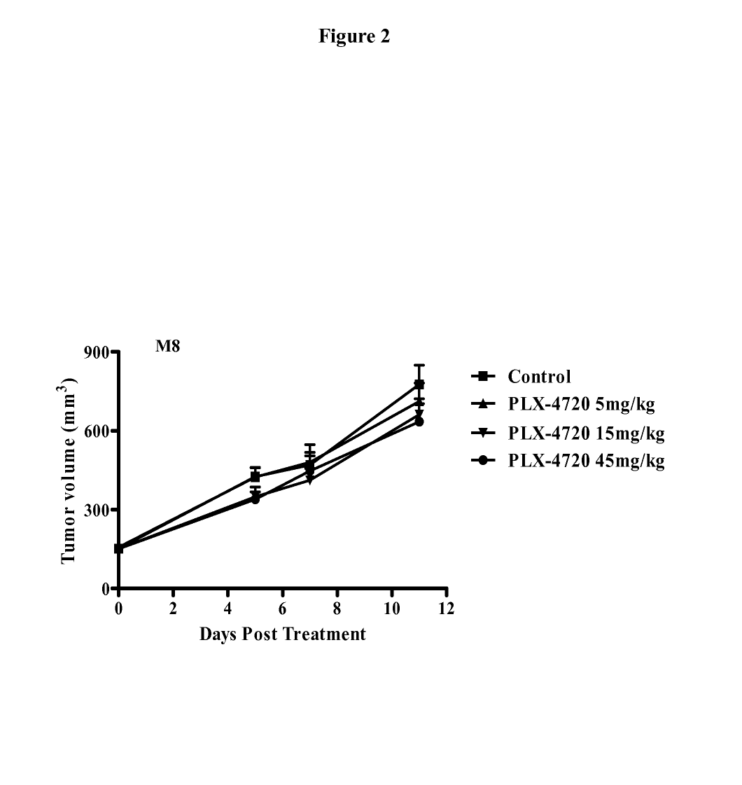

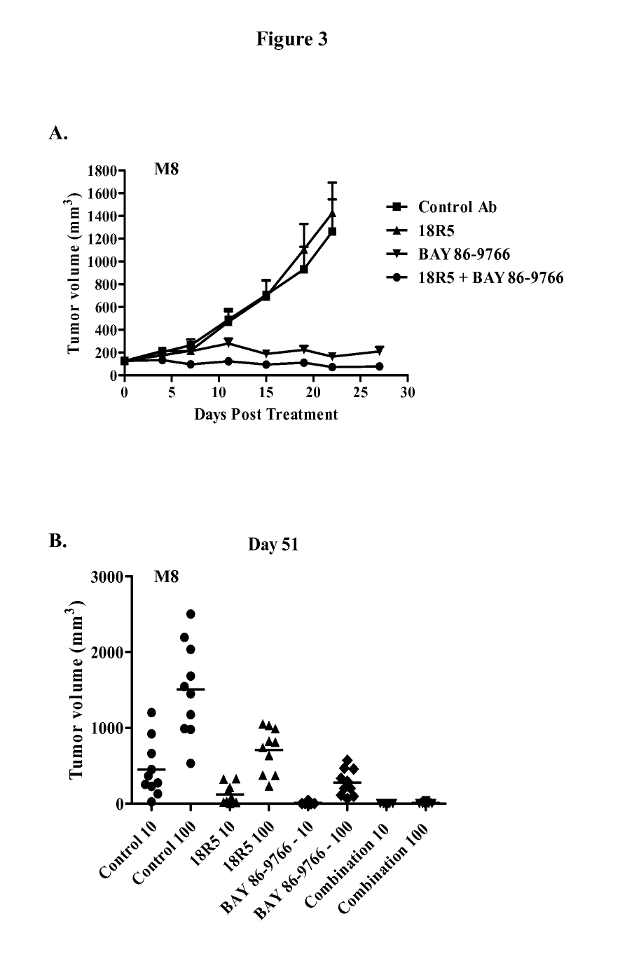

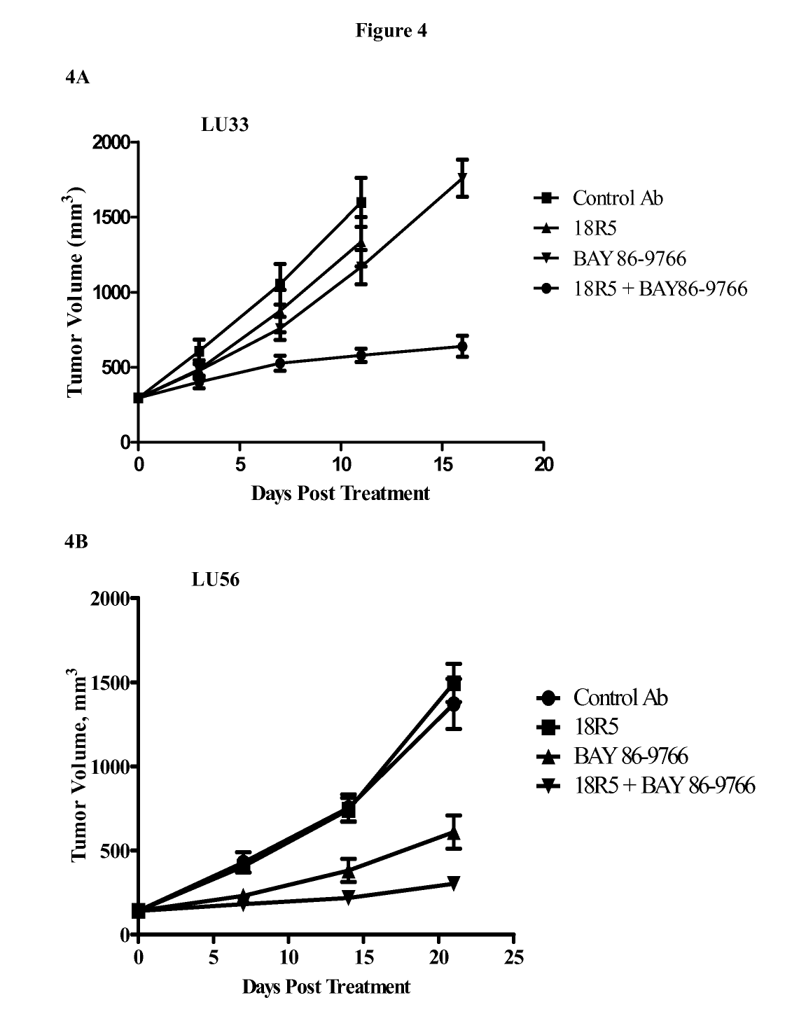

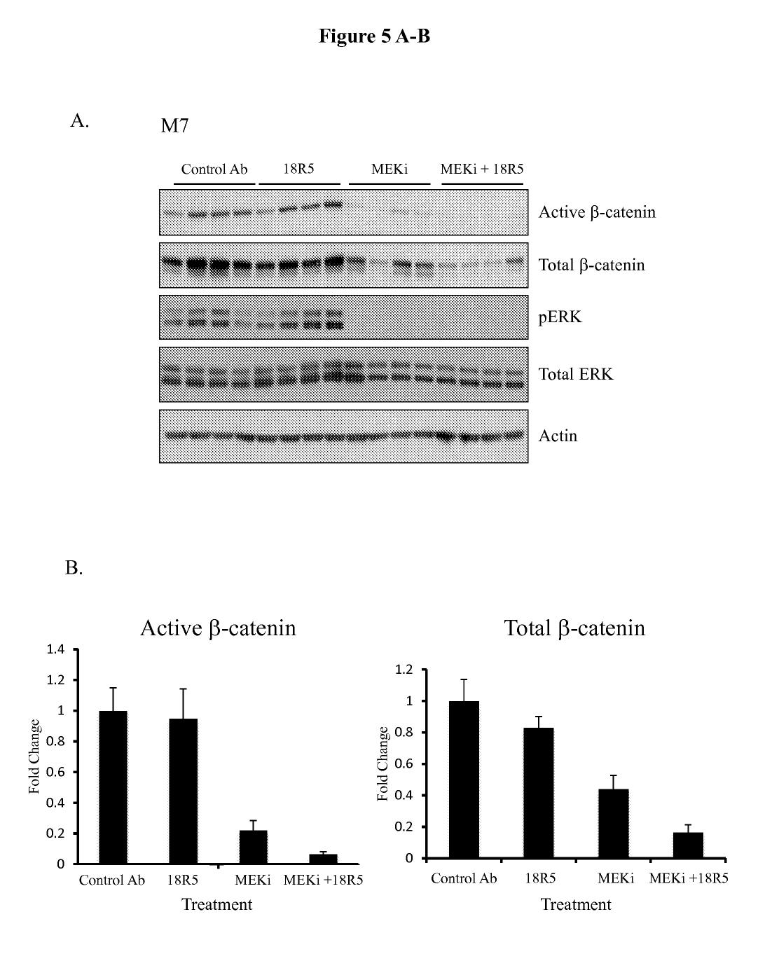

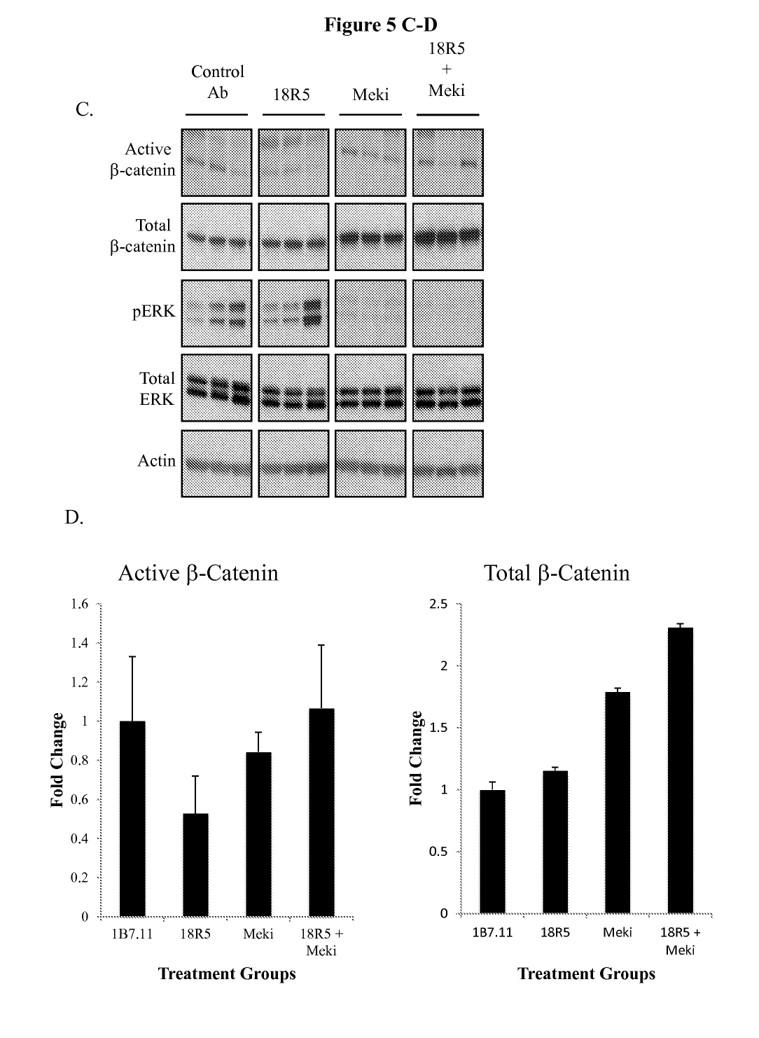

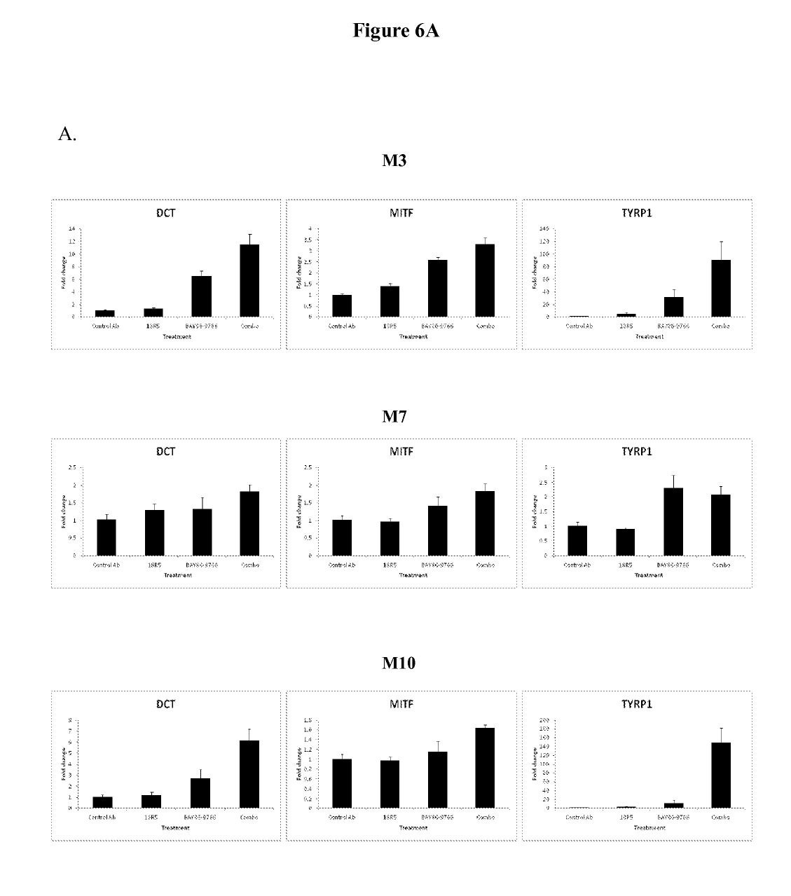

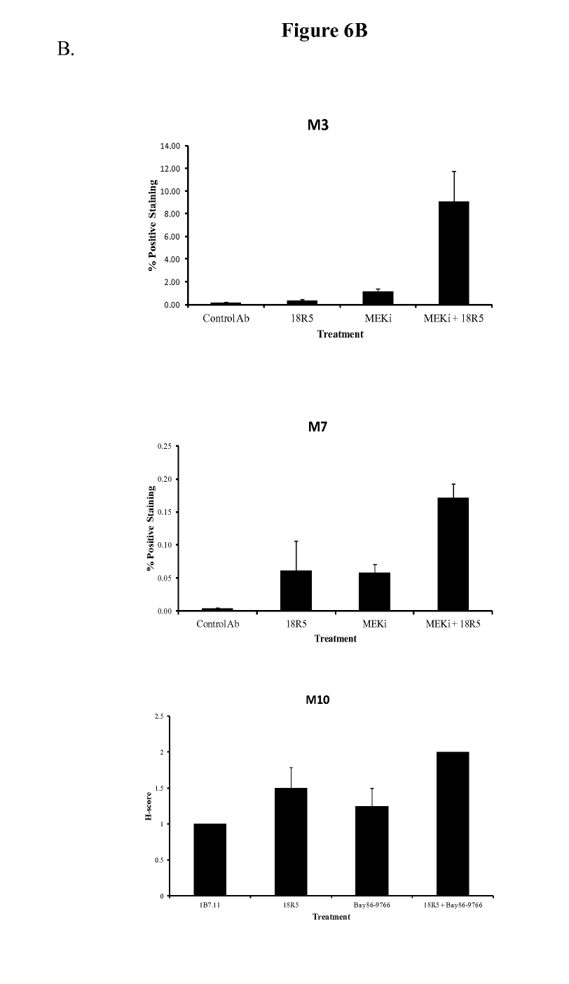

A number of melanoma tumors were established in a xenograft model and were evaluated for B-Raf, N-Ras, and K-Ras mutations (Example 1). Treatment with a pan-FZD antibody in combination with a MEK inhibitor was shown to reduce growth of melanoma tumors (Examples 2 and 3; FIGS. 1 and 3) and lung tumors (Examples 4 and 7; FIG. 4). Treatment with a pan-FZD antibody in combination with a MEK inhibitor was shown to reduce growth of a melanoma tumor that was resistant to a B-Raf inhibitor (Example 3 and FIGS. 2 and 3). It was shown that treatment with a pan-FZD antibody in combination with a MEK inhibitor reduced tumorigenicity of melanoma cells (Example 3 and FIG. 3). Treatment with a pan-FZD antibody in combination with a MEK inhibitor was shown to decrease active and total β-catenin in a melanoma tumor comprising an N-Ras mutation (Example 5 and FIG. 5). Treatment with a pan-FZD antibody in combination with a MEK inhibitor was shown to increase expression of melanocyte lineage genes and to increased levels of E-cadherin protein in melanoma tumors (Example 6 and FIG. 6). These examples support the hypothesis that combination treatment comprising a Wnt pathway inhibitor and a MAPK pathway inhibitor targeted both tumor cells and cancer stem cells, resulting in reduced tumor growth, decreased cancer stem cell frequency, and differentiation of tumorigenic cells.

### I. DEFINITIONS

To facilitate an understanding of the present invention, a number of terms and phrases are defined below.

The terms “antagonist” and “antagonistic” as used herein refer to any molecule that partially or fully blocks, inhibits, reduces, or neutralizes a biological activity of a target and/or signaling pathway (e.g., the Wnt pathway or the MAPK pathway). The term “antagonist” is used herein to include any molecule that partially or fully blocks, inhibits, reduces, or neutralizes the activity of a protein (e.g., a FZD protein or a Wnt protein). Suitable antagonist molecules specifically include, but are not limited to, antagonist antibodies, antibody fragments, soluble receptors, or small molecules.

The term “antibody” as used herein refers to an immunoglobulin molecule that recognizes and specifically binds a target, such as a protein, polypeptide, peptide, carbohydrate, polynucleotide, lipid, or combinations of the foregoing, through at least one antigen-binding site within the variable region of the immunoglobulin molecule. As used herein, the term encompasses intact polyclonal antibodies, intact monoclonal antibodies, antibody fragments comprising an antigen-binding site (such as Fab, Fab′, F(ab′)2, and Fv fragments), single chain Fv (scFv) antibodies, multispecific antibodies such as bispecific antibodies generated from at least two intact antibodies, monospecific antibodies, monovalent antibodies, chimeric antibodies, humanized antibodies, human antibodies, fusion proteins comprising an antigen determination portion of an antibody, and any other modified immunoglobulin molecule comprising an antigen-binding site as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes.

The term “antibody fragment” refers to a portion of an intact antibody and refers to the antigenic determining variable region or antigen-binding site of an intact antibody. Examples of antibody fragments include, but are not limited to, Fab, Fab′, F(ab′)2, and Fv fragments, linear antibodies, single chain antibodies, and multispecific antibodies formed from antibody fragments. “Antibody fragment” as used herein comprises at least one antigen-binding site or epitope-binding site.

The term “variable region” of an antibody refers to the variable region of the antibody light chain, or the variable region of the antibody heavy chain, either alone or in combination. The variable region of the heavy and light chain each consist of four framework regions (FR) connected by three complementarity determining regions (CDRs), also known as “hypervariable regions”. The CDRs in each chain are held together in close proximity by the framework regions and, with the CDRs from the other chain, contribute to the formation of the antigen-binding site of the antibody. There are at least two techniques for determining CDRs: (1) an approach based on cross-species sequence variability (i.e., Kabat et al., 1991, Sequences of Proteins of Immunological Interest, 5th Edition, National Institutes of Health, Bethesda Md.), and (2) an approach based on crystallographic studies of antigen-antibody complexes (Al-Lazikani et al., 1997, J. Mol. Biol., 273:927-948). In addition, combinations of these two approaches are sometimes used in the art to determine CDRs.

The term “monoclonal antibody” as used herein refers to a homogenous antibody population involved in the highly specific recognition and binding of a single antigenic determinant or epitope. This is in contrast to polyclonal antibodies that typically include a mixture of different antibodies directed against different antigenic determinants. The term “monoclonal antibody” encompasses both intact and full-length monoclonal antibodies as well as antibody fragments (e.g., Fab, Fab′, F(ab′)2, Fv), single chain (scFv) antibodies, fusion proteins comprising an antibody portion, and any other modified immunoglobulin molecule comprising at least one antigen-binding site. Furthermore, “monoclonal antibody” refers to such antibodies made by any number of techniques, including but not limited to, hybridoma production, phage selection, recombinant expression, and transgenic animals.

The term “humanized antibody” as used herein refers to antibodies that are specific immunoglobulin chains, chimeric immunoglobulins, or fragments thereof that contain minimal non-human sequences. Typically, humanized antibodies are human immunoglobulins in which residues of the CDRs are replaced by residues from the CDRs of a non-human species (e.g., mouse, rat, rabbit, or hamster) that have the desired specificity, affinity, and/or binding capability (Jones et al., 1986, Nature, 321:522-525; Riechmann et al., 1988, Nature, 332:323-327; Verhoeyen et al., 1988, Science, 239:1534-1536).

The term “human antibody” as used herein refers to an antibody produced by a human or an antibody having an amino acid sequence corresponding to an antibody produced by a human made using any of the techniques known in the art. This definition of a human antibody specifically excludes a humanized antibody comprising non-human antigen-binding residues.

The term “chimeric antibody” as used herein refers to an antibody wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and/or binding capability, while the constant regions are homologous to the sequences in antibodies derived from another species (usually human) to avoid eliciting an immune response in that species.

The phrase “affinity-matured antibody” as used herein refers to an antibody with one or more alterations in one or more CDRs that result in an improvement in the affinity of the antibody for antigen, compared to a parent antibody that does not possess those alterations(s). Preferred affinity-matured antibodies will have nanomolar or even picomolar affinities for the target antigen. Affinity-matured antibodies are produced by procedures known in the art. For example, Marks et al., 1992, Bio/Technology 10:779-783, describes affinity maturation by VH and VL domain shuffling. Random mutagenesis of CDR and/or framework residues is described by Barbas et al., 1994, PNAS, 91:3809-3813; Schier et al., 1995, Gene, 169:147-155; Yelton et al., 1995, J. Immunol. 155:1994-2004; Jackson et al., 1995, J. Immunol., 154:3310-9; and Hawkins et al., 1992, J. Mol. Biol., 226:889-896.

The terms “epitope” and “antigenic determinant” are used interchangeably herein and refer to that portion of an antigen capable of being recognized and specifically bound by a particular antibody. When the antigen is a polypeptide, epitopes can be formed both from contiguous amino acids and non-contiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, or 8-10 amino acids in a unique spatial conformation.

The terms “selectively binds” or “specifically binds” mean that a binding agent or an antibody reacts or associates more frequently, more rapidly, with greater duration, with greater affinity, or with some combination of the above to the epitope, protein, or target molecule than with alternative substances, including unrelated or related proteins. In certain embodiments “specifically binds” means, for instance, that an antibody binds a target with a K_{D} of about 0.1 mM or less, but more usually less than about 1 μM. In certain embodiments, “specifically binds” means that an antibody binds a target with a K_{D} of at least about 0.1 μM or less, at least about 0.01 μM or less, or at least about 1 nM or less. Because of the sequence identity between homologous proteins in different species, specific binding can include an antibody that recognizes a protein in more than one species (e.g., human FZD protein and mouse FZD protein). Likewise, because of homology within certain regions of polypeptide sequences of different proteins, specific binding can include an antibody (or other polypeptide or binding agent) that recognizes more than one protein (e.g., human FZD2 and human FZD7). It is understood that, in certain embodiments, an antibody or binding agent that specifically binds a first target may or may not specifically bind a second target. As such, “specific binding” does not necessarily require (although it can include) exclusive binding, i.e. binding to a single target. Thus, an antibody may, in certain embodiments, specifically bind more than one target. In certain embodiments, multiple targets may be bound by the same antigen-binding site on the antibody. For example, an antibody may, in certain instances, comprise two identical antigen-binding sites, each of which specifically binds the same epitope on two or more proteins (e.g., FZD2 and FZD7). In certain alternative embodiments, an antibody may be bispecific and comprise at least two antigen-binding sites with differing specificities. By way of non-limiting example, a bispecific antibody may comprise one antigen-binding site that recognizes an epitope on one protein (e.g., a human FZD protein) and further comprise a second, different antigen-binding site that recognizes a different epitope on a second protein. Generally, but not necessarily, reference to binding means specific binding.

As used herein the term “soluble receptor” refers to an N-terminal extracellular fragment (or a portion thereof) of a receptor protein preceding the first transmembrane domain of the receptor that can be secreted from a cell in soluble form.

As used herein the term “FZD soluble receptor” refers to an N-terminal extracellular fragment of a FZD receptor protein preceding the first transmembrane domain of the receptor that can be secreted from a cell in soluble form, FZD soluble receptors comprising the entire N-terminal extracellular domain (ECD) as well as smaller fragments are encompassed by the term. Thus, FZD soluble receptors comprising the Fri domain are also included in this term.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides of this invention may be based upon antibodies, in certain embodiments, the polypeptides can occur as single chains or associated chains.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. The phrase “amino acid analog” refers to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but retain the same basic chemical structure as a naturally occurring amino acid. The phrase “amino acid mimetic” refers to chemical compounds that have a structure that is different from the general chemical structure of an amino acid, but that function similarly to a naturally occurring amino acid.

The terms “polynucleotide” and “nucleic acid” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST and BLAST variations, ALIGN and ALIGN variations, Megalign, BestFit, GCG Wisconsin Package, etc. In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 nucleotides or residues, at least about 60-80 nucleotides or residues in length or any integral value therebetween. In some embodiments, identity exists over a longer region than 60-80 nucleotides or residues, such as at least about 80-100 nucleotides or residues, and in some embodiments the sequences are substantially identical over the full length of the sequences being compared, such as the coding region of a nucleotide sequence.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Preferably, conservative substitutions in the sequences of the polypeptides and antibodies of the invention do not abrogate the binding of the polypeptide or antibody containing the amino acid sequence to the antigen(s). Methods of identifying nucleotide and amino acid conservative substitutions which do not eliminate antigen binding are well-known in the art.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells, or compositions include those which have been purified to a degree that they are no longer in a form in which they are found in nature. In some embodiments, a polypeptide, antibody, polynucleotide, vector, cell, or composition which is isolated is substantially pure.

The term “substantially pure” as used herein refers to material which is at least 50% pure (i.e., free from contaminants), at least 90% pure, at least 95% pure, at least 98% pure, or at least 99% pure.

The terms “cancer” and “cancerous” as used herein refer to or describe the physiological condition in mammals in which a population of cells is characterized by unregulated cell growth. Examples of cancer include, but are not limited to, carcinoma, blastoma, sarcoma, and hematologic cancers such as lymphoma and leukemia.

The terms “proliferative disorder” and “proliferative disease” refer to disorders associated with abnormal cell proliferation such as cancer.

The terms “tumor” and “neoplasm” as used herein refer to any mass of tissue that results from excessive cell growth or proliferation, either benign (non-cancerous) or malignant (cancerous), including pre-cancerous lesions.

The term “metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates from the primary site of disease to invade neighboring body structures.

The terms “cancer stem cell” and “CSC” and “tumor stem cell” and “tumor initiating cell” are used interchangeably herein and refer to cells from a cancer or tumor that: (1) have extensive proliferative capacity; 2) are capable of asymmetric cell division to generate one or more types of differentiated cell progeny wherein the differentiated cells have reduced proliferative or developmental potential; and (3) are capable of symmetric cell divisions for self-renewal or self-maintenance. These properties confer on the cancer stem cells the ability to form or establish a tumor or cancer upon serial transplantation into an immunocompromised host (e.g., a mouse) compared to the majority of tumor cells that fail to form tumors. Cancer stem cells undergo self-renewal versus differentiation in a chaotic manner to form tumors with abnormal cell types that can change over time as mutations occur.

The terms “cancer cell” and “tumor cell” refer to the total population of cells derived from a cancer or tumor or pre-cancerous lesion, including both non-tumorigenic cells, which comprise the bulk of the cancer cell population, and tumorigenic cells (cancer stem cells). As used herein, the terms “cancer cell” or “tumor cell” will be modified by the term “non-tumorigenic” when referring solely to those cells lacking the capacity to renew and differentiate to distinguish those tumor cells from cancer stem cells.

The term “tumorigenic” as used herein refers to the functional features of a cancer stem cell including the properties of self-renewal (giving rise to additional tumorigenic cancer stem cells) and proliferation to generate all other tumor cells (giving rise to differentiated and thus non-tumorigenic tumor cells).

The term “tumorigenicity” as used herein refers to the ability of a sample of cells from a tumor to form palpable tumors upon serial transplantation into immunocompromised hosts (e.g., mice).

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

As used herein the term “inhibit tumor growth” refers to any mechanism by which tumor growth can be inhibited. In certain embodiments, tumor growth is inhibited by slowing proliferation of tumor cells. In certain embodiments, tumor growth is inhibited by halting proliferation of tumor cells. In certain embodiments, tumor growth is inhibited by killing tumor cells. In some embodiments, tumor growth is inhibited by reducing the frequency of cancer stem cells. In certain embodiments, tumor growth is inhibited by reducing the number of cancer stem cells. In certain embodiments, tumor growth is inhibited by inducing apoptosis of tumor cells. In certain embodiments, tumor growth is inhibited by inducing differentiation of tumor cells. In certain embodiments, tumor growth is inhibited by depriving tumor cells of nutrients. In certain embodiments, tumor growth is inhibited by preventing migration of tumor cells. In certain embodiments, tumor growth is inhibited by preventing invasion of tumor cells.

The term “pharmaceutically acceptable” refers to an agent, compound, molecule, etc. approved or approvable by a regulatory agency of the Federal or a state government or listed in the U.S. Pharmacopeia or other generally recognized pharmacopeia for use in animals, including humans.

The phrases “pharmaceutically acceptable excipient, carrier or adjuvant” and “acceptable pharmaceutical carrier” refer to an excipient, carrier, or adjuvant that can be administered to a subject, together with at least one binding agent (e.g., an antibody) of the present disclosure, and which does not destroy the pharmacological activity thereof and is nontoxic when administered in doses sufficient to deliver a therapeutic effect.

The terms “effective amount” and “therapeutically effective amount” and “therapeutic effect” refer to an amount of a binding agent, an antibody, polypeptide, polynucleotide, small molecule, or other drug effective to “treat” a disease or disorder in a subject or mammal. In the case of cancer, the therapeutically effective amount of a drug (e.g., an antibody) has a therapeutic effect and as such can reduce the number of cancer cells; decrease tumorigenicity, tumorigenic frequency, or tumorigenic capacity; reduce the number or frequency of cancer stem cells; reduce tumor size; reduce the cancer cell population; inhibit and/or stop cancer cell infiltration into peripheral organs including, for example, the spread of cancer into soft tissue and bone; inhibit and stop tumor or cancer cell metastasis; inhibit and/or stop tumor or cancer cell growth; relieve to some extent one or more of the symptoms associated with the cancer; reduce morbidity and mortality; improve quality of life; or a combination of such effects. To the extent the agent, for example an antibody, prevents growth and/or kills existing cancer cells, it can be referred to as cytostatic and/or cytotoxic.

The terms “treating” and “treatment” and “to treat” and “alleviating” and “to alleviate” refer to both 1) therapeutic measures that cure, slow down, lessen symptoms of, and/or halt progression of a diagnosed pathologic condition or disorder and 2) prophylactic or preventative measures that prevent or slow the development of a targeted pathologic condition or disorder. Thus, those in need of treatment include those who already have a disorder; those prone to have a disorder; and those in whom a disorder is to be prevented. In some embodiments, a subject is successfully “treated” according to the methods of the present invention if the patient shows one or more of the following: a reduction in the number of or complete absence of cancer cells; a reduction in tumor size; inhibition of or an absence of cancer cell infiltration into peripheral organs including the spread of cancer cells into soft tissue and bone; inhibition of or an absence of tumor or cancer cell metastasis; inhibition or an absence of cancer growth; relief of one or more symptoms associated with the specific cancer, reduced morbidity and mortality; improvement in quality of life; reduction in tumorigenicity; reduction in the number or frequency of cancer stem cells; or some combination of effects.

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. WNT PATHWAY INHIBITORS

The present invention provides Wnt pathway inhibitors for use in methods of inhibiting tumor growth or for use in methods of treating cancer, particularly in combination with MAPK pathway inhibitors.

In certain embodiments, the Wnt pathway inhibitors are agents that bind one or more human frizzled proteins (FZD). These agents are referred to herein as “FZD-binding agents”. In some embodiments, the FZD-binding agents specifically bind one, two, three, four, five, six, seven, eight, nine, or ten FZD proteins. In some embodiments, the FZD-binding agent binds one or more FZD proteins selected from the group consisting of FZD1, FZD2, FZD3, FZD4, FZD5, FZD6, FZD7, FZD8, FZD9, and FZD10. In some embodiments, FZD-binding agent binds one or more FZD proteins comprising FZD1, FZD2, FZD5, FZD7, and/or FZD8. In certain embodiments, FZD-binding agent binds FZD7. In certain embodiments, FZD-binding agent binds FZD5 and/or FZD8. In certain embodiments, the FZD-binding agent specifically binds FZD1, FZD2, FZD5, FZD7, and FZD8. Non-limiting examples of FZD-binding agents can be found in U.S. Pat. No. 7,982,013, which is incorporated by reference herein in its entirety.

In certain embodiments, the FZD-binding agent is a FZD antagonist. In certain embodiments, the FZD-binding agent is a Wnt pathway antagonist. In certain embodiments, the FZD-binding agent inhibits Wnt signaling. In some embodiments, the FZD-binding agent inhibits canonical Wnt signaling.

In some embodiments, the FZD-binding agents are antibodies. In some embodiments, the FZD-binding agents are polypeptides. In certain embodiments, the FZD-binding agent is an antibody or a polypeptide comprising an antigen-binding site. In certain embodiments, an antigen-binding site of a FZD-binding antibody or polypeptide described herein is capable of binding (or binds) one, two, three, four, five, or more human FZD proteins. In certain embodiments, an antigen-binding site of the FZD-binding antibody or polypeptide is capable of specifically binding one, two, three, four, or five human FZD proteins selected from the group consisting of FZD1, FZD2, FZD3, FZD4, FZD5, FZD6, FZD7, FZD8, FZD9 and FZD10. In some embodiments, when the FZD-binding agent is an antibody that binds more than one FZD protein, it may be referred to as a “pan-FZD antibody”.

In certain embodiments, the FZD-binding agent (e.g., antibody) specifically binds the extracellular domain (ECD) within the one or more human FZD proteins to which it binds. In certain embodiments, the FZD-binding agent specifically binds within the Fri domain (also known as the cysteine-rich domain (CRD)) of the human FZD protein to which it binds. Sequences of the Fri domain of each of the human FZD protein are known in the art and are provided as SEQ ID NO:11 (FZD1), SEQ ID NO:12 (FZD2), SEQ ID NO:13 (FZD3), SEQ ID NO:14 (FZD4), SEQ ID NO:15 (FZD5), SEQ ID NO:16 (FZD6), SEQ ID NO:17 (FZD7), SEQ ID NO:18 (FZD), SEQ ID NO:19 (FZD9), and SEQ ID NO:20 (FZD10).

In certain embodiments, the FZD-binding agent binds one, two, three, four, five, or more FZD proteins. In some embodiments, the FZD-binding agent specifically binds one, two, three, four, or five FZD proteins selected from the group consisting of FZD1, FZD2, FZD5, FZD7, and FZD8. In some embodiments, the FZD-binding agent specifically binds at least FZD5 and FZD8.

In some embodiments, the FZD-binding agent binds at least one human FZD protein with a dissociation constant (K_{D}) of about 1 μM or less, about 100 nM or less, about 40 nM or less, about 20 nM or less, about 10 nM or less, about 1 nM or less, or about 0.1 nM or less. In some embodiments, a FZD-binding agent binds at least one FZD protein with a K_{D} of about 1 nM or less. In some embodiments, a FZD-binding agent binds at least one FZD protein with a K_{D} of about 0.1 nM or less. In certain embodiments, a FZD-binding agent binds each of one or more (e.g., 1, 2, 3, 4, or 5) of FZD1, FZD2, FZD5, FZD7, and FZD8 with a K_{D} of about 40 nM or less. In certain embodiments, the FZD-binding agent binds to each of one or more of FZD1, FZD2, FZD5, FZD7, and FZD8 with a K_{D} of about 10 nM or less. In certain embodiments, the FZD-binding agent binds each of FZD1, FZD2, FZD5, FZD7, and FZD8 with a K_{D} of about 10 nM. In some embodiments, the K_{D} of the binding agent (e.g., an antibody) to a FZD protein is the K_{D} determined using a FZD-Fc fusion protein comprising at least a portion of the FZD extracellular domain or FZD-Fri domain immobilized on a Biacore chip.

In certain embodiments, the FZD-binding agent binds one or more (for example, two or more, three or more, or four or more) human FZD proteins with an EC₅₀ of about 1 μM or less, about 100 nM or less, about 40 nM or less, about 20 nM or less, about 10 nM or less, or about 1 nM or less. In certain embodiments, a FZD-binding agent binds to more than one FZD protein with an EC₅₀ of about 40 nM or less, about 20 nM or less, or about 10 nM or less. In certain embodiments, the FZD-binding agent has an EC₅₀ of about 20 nM or less with respect to one or more (e.g., 1, 2, 3, 4, or 5) of the following FZD proteins: FZD1, FZD2, FZD5, FZD7, and FZD8. In certain embodiments, the FZD-binding agent has an EC₅₀ of about 10 nM or less with respect to one or more (e.g., 1, 2, 3, 4, or 5) of the following FZD proteins: FZD1, FZD2, FZD5, FZD7, and FZD8. In certain embodiments, the FZD-binding agent has an EC₅₀ of about 40 nM or less or 20 nM or less with respect to binding of FZD5 and/or FZD8.

In certain embodiments, the Wnt pathway inhibitor is a FZD-binding agent which is an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In certain embodiments, the antibody is an IgG1 antibody. In certain embodiments, the antibody is an IgG2 antibody. In certain embodiments, the antibody is an antibody fragment comprising an antigen-binding site. In some embodiments, the antibody is monovalent, monospecific, bivalent, bispecific, or multispecific. In some embodiments, the antibody is conjugated to a cytotoxic moiety. In some embodiments, the antibody is isolated. In some embodiments, the antibody is substantially pure.

The FZD-binding agents (e.g., antibodies) of the present invention can be assayed for specific binding by any method known in the art. The immunoassays which can be used include, but are not limited to, competitive and non-competitive assay systems using techniques such as Biacore analysis, FACS analysis, immunofluorescence, immunocytochemistry, Western blots, radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, and protein A immunoassays. Such assays are routine and well-known in the art (see, e.g., Ausubel et al., Editors, 1994-present, Current Protocols in Molecular Biology, John Wiley & Sons, Inc., New York, N.Y.).

For example, the specific binding of an antibody to a human FZD protein may be determined using ELISA. An ELISA assay comprises preparing antigen, coating wells of a 96 well microtiter plate with antigen, adding the FZD-binding agent (e.g., an antibody) conjugated to a detectable compound such as an enzymatic substrate (e.g. horseradish peroxidase or alkaline phosphatase) to the well, incubating for a period of time and detecting the presence of the FZD-binding agent bound to the antigen. In some embodiments, the FZD-binding antibody or agent is not conjugated to a detectable compound, but instead a second conjugated antibody that recognizes the FZD-binding antibody or agent is added to the well. In some embodiments, instead of coating the well with the antigen, the FZD-binding antibody or agent can be coated to the well and a second antibody conjugated to a detectable compound can be added following the addition of the antigen to the coated well. One of skill in the art would be knowledgeable as to the parameters that can be modified to increase the signal detected as well as other variations of ELISAs that may be used.

In another example, the specific binding of an antibody to a human FZD protein may be determined using FACS. A FACS screening assay may comprise generating a cDNA construct that expresses an antigen as a fusion protein, transfecting the construct into cells, expressing the antigen on the surface of the cells, mixing the FZD-binding antibody or other FZD-binding agent with the transfected cells, and incubating for a period of time. The cells bound by the FZD-binding antibody or other FZD-binding agent may be identified by using a secondary antibody conjugated to a detectable compound (e.g., PE-conjugated anti-Fc antibody) and a flow cytometer. One of skill in the art would be knowledgeable as to the parameters that can be modified to optimize the signal detected as well as other variations of FACS that may enhance screening (e.g., screening for blocking antibodies).

The binding affinity of an antibody or other binding-agent to an antigen (e.g., a FZD protein) and the off-rate of an antibody-antigen interaction can be determined by competitive binding assays. One example of a competitive binding assay is a radioimmunoassay comprising the incubation of labeled antigen (e.g., ³H or ¹²⁵I), or fragment or variant thereof, with the antibody of interest in the presence of increasing amounts of unlabeled antigen followed by the detection of the antibody bound to the labeled antigen. The affinity of the antibody for an antigen (e.g., a FZD protein) and the binding off-rates can be determined from the data by Scatchard plot analysis. In some embodiments, Biacore kinetic analysis is used to determine the binding on and off rates of antibodies or agents that bind an antigen (e.g., a FZD protein). Biacore kinetic analysis comprises analyzing the binding and dissociation of antibodies from chips with immobilized antigen (e.g., a FZD protein) on their surface.

In certain embodiments, the invention provides a Wnt pathway inhibitor which is a FZD-binding agent (e.g., an antibody) that comprises a heavy chain CDR1 comprising GFTFSHYTLS (SEQ ID NO:5), a heavy chain CDR2 comprising VISGDGSYTYYADSVKG (SEQ ID NO:6), and a heavy chain CDR3 comprising NFIKYVFAN (SEQ ID NO:7). In some embodiments, the FZD-binding agent further comprises a light chain CDR1 comprising SGDNIGSFYVH (SEQ ID NO:8), a light chain CDR2 comprising DKSNRPSG (SEQ ID NO:9), and a light chain CDR3 comprising QSYANTLSL (SEQ ID NO:10). In some embodiments, the FZD-binding agent comprises a light chain CDR1 comprising SGDNIGSFYVH (SEQ ID NO:8), a light chain CDR2 comprising DKSNRPSG (SEQ ID NO:9), and a light chain CDR3 comprising QSYANTLSL (SEQ ID NO: 10). In certain embodiments, the FZD-binding agent comprises: (a) a heavy chain CDR1 comprising GFTFSHYTLS (SEQ ID NO:5), a heavy chain CDR2 comprising VISGDGSYTYYADSVKG (SEQ ID NO:6), and a heavy chain CDR3 comprising NFIKYVFAN (SEQ ID NO:7), and (b) a light chain CDR1 comprising SGDNIGSFYVH (SEQ ID NO:8), a light chain CDR2 comprising DKSNRPSG (SEQ ID NO:9), and a light chain CDR3 comprising QSYANTLSL (SEQ ID NO:10).

In certain embodiments, the invention provides a FZD-binding agent (e.g., an antibody) that comprises: (a) a heavy chain CDR1 comprising GFTFSHYTLS (SEQ ID NO:5), or a variant thereof comprising 1, 2, 3, or 4 amino acid substitutions; (b) a heavy chain CDR2 comprising VISGDGSYTYYADSVKG (SEQ ID NO:6), or a variant thereof comprising 1, 2, 3, or 4 amino acid substitutions; (c) a heavy chain CDR3 comprising NFIKYVFAN (SEQ ID NO:7), or a variant thereof comprising 1, 2, 3, or 4 amino acid substitutions; (d) a light chain CDR1 comprising SGDNIGSFYVH (SEQ ID NO:8), or a variant thereof comprising 1, 2, 3, or 4 amino acid substitutions; (e) a light chain CDR2 comprising DKSNRPSG (SEQ ID NO:9), or a variant thereof comprising 1, 2, 3, or 4 amino acid substitutions; and (f) a light chain CDR3 comprising QSYANTLSL (SEQ ID NO: 10), or a variant thereof comprising 1, 2, 3, or 4 amino acid substitutions. In certain embodiments, the amino acid substitutions are conservative substitutions.

In certain embodiments, the invention provides a FZD-binding agent (e.g., an antibody) that comprises a heavy chain variable region having at least about 80% sequence identity to SEQ ID NO:3, and/or a light chain variable region having at least 80% sequence identity to SEQ ID NO:4. In certain embodiments, the FZD-binding agent comprises a heavy chain variable region having at least about 85%, at least about 90%, at least about 95%, at least about 97%, or at least about 99% sequence identity to SEQ ID NO:3. In certain embodiments, the FZD-binding agent comprises a light chain variable region having at least about 85%, at least about 90%, at least about 95%, at least about 97%, or at least about 99% sequence identity to SEQ ID NO:4. In certain embodiments, the FZD-binding agent comprises a heavy chain variable region having at least about 95% sequence identity to SEQ ID NO:3, and/or a light chain variable region having at least about 95% sequence identity to SEQ ID NO:4. In certain embodiments, the FZD-binding agent comprises a heavy chain variable region comprising SEQ ID NO:3, and/or a light chain variable region comprising SEQ ID NO:4. In certain embodiments, the FZD-binding agent comprises a heavy chain variable region consisting essentially of SEQ ID NO:3, and a light chain variable region consisting essentially of SEQ ID NO:4.

In certain embodiments, the invention provides a FZD-binding agent (e.g., an antibody) that comprises: (a) a heavy chain having at least 90% sequence identity to SEQ ID NO:1 (with or without the signal sequence) or SEQ ID NO:60; and/or (b) a light chain having at least 90% sequence identity to SEQ ID NO:2 (with or without the signal sequence) or SEQ ID NO:61. In some embodiments, the FZD-binding agent comprises: (a) a heavy chain having at least 95% sequence identity to SEQ ID NO: 1 (with or without the signal sequence) or SEQ ID NO:60; and/or (b) a light chain having at least 95% sequence identity to SEQ ID NO:2 (with or without the signal sequence) or SEQ ID NO:61. In some embodiments, the FZD-binding agent comprises a heavy chain comprising SEQ ID NO:1 (with or without the signal sequence) or SEQ ID NO:60, and/or a light chain comprising SEQ ID NO:2 (with or without the signal sequence) or SEQ ID NO:61. In some embodiments, the FZD-binding agent comprises a heavy chain consisting essentially of amino acids 20-463 of SEQ ID NO:1, and a light chain consisting essentially of amino acids 20-232 of SEQ ID NO:2. In some embodiments, the FZD-binding agent comprises a heavy chain consisting essentially of SEQ ID NO:60, and a light chain consisting essentially of SEQ ID NO:61.

In certain embodiments, the invention provides a Wnt pathway inhibitor which is a FZD-binding agent (e.g., an antibody) that specifically binds at least one of FZD1, FZD2, FZD5, FZD7 and/or FZD8, wherein the FZD-binding agent (e.g., an antibody) comprises one, two, three, four, five, and/or six of the CDRs of antibody 18R5. Antibody 18R5, as well as other FZD-binding agents, has been previously described in U.S. Pat. No. 7,982,013. DNA encoding the heavy chains and light chains of the 18R5 IgG2 antibody was deposited with the ATCC, under the conditions of the Budapest Treaty on Sep. 29, 2008, and assigned ATCC deposit designation number PTA-9541. In some embodiments, the FZD-binding agent comprises one or more of the CDRs of 18R5, two or more of the CDRs of 18R5, three or more of the CDRs of 18R5, four or more of the CDRs of 18R5, five or more of the CDRs of 18R5, or all six of the CDRs of 18R5.

The invention provides polypeptides which are Wnt pathway inhibitors. The polypeptides include, but are not limited to, antibodies that specifically bind human FZD proteins. In some embodiments, a polypeptide binds one or more FZD proteins selected from the group consisting of FZD1, FZD2, FZD3, FZD4, FZD5, FZD6, FZD7, FZD8, FZD9, and FZD10. In some embodiments, a polypeptide binds FZD1, FZD2, FZD5, FZD7, and/or FZD8. In some embodiments, a polypeptide binds FZD1, FZD2, FZD5, FZD7, and FZD8.

In certain embodiments, a polypeptide comprises one, two, three, four, five, and/or six of the CDRs of antibody 18R5. In some embodiments, a polypeptide comprises CDRs with up to four (i.e., 0, 1, 2, 3, or 4) amino acid substitutions per CDR. In certain embodiments, the heavy chain CDR(s) are contained within a heavy chain variable region. In certain embodiments, the light chain CDR(s) are contained within a light chain variable region.

In some embodiments, the invention provides a polypeptide that specifically binds one or more human FZD proteins, wherein the polypeptide comprises an amino acid sequence having at least about 80% sequence identity to SEQ ID NO:3, and/or an amino acid sequence having at least about 80% sequence identity to SEQ ID NO:4. In certain embodiments, the polypeptide comprises an amino acid sequence having at least about 85%, at least about 90%, at least about 95%, at least about 97%, or at least about 99% sequence identity to SEQ ID NO:3. In certain embodiments, the polypeptide comprises an amino acid sequence having at least about 85%, at least about 90%, at least about 95%, at least about 97%, or at least about 99% sequence identity to SEQ ID NO:4. In certain embodiments, the polypeptide comprises an amino acid sequence having at least about 95% sequence identity to SEQ ID NO:3, and/or an amino acid sequence having at least about 95% sequence identity to SEQ ID NO:4. In certain embodiments, the polypeptide comprises an amino acid sequence comprising SEQ ID NO:3, and/or an amino acid sequence comprising SEQ ID NO:4.

In some embodiments, a FZD-binding agent comprises a polypeptide comprising a sequence selected from the group consisting of: SEQ ID NO:1, SEQ ID NO:2, SEQ ID NO:3, SEQ ID NO:4, SEQ ID NO:60, and SEQ ID NO:61.

In certain embodiments, a FZD-binding agent comprises the heavy chain variable region and light chain variable region of the 18R5 antibody. In certain embodiments, a FZD-binding agent comprises the heavy chain and light chain of the 18R5 antibody (with or without the leader sequence).

In certain embodiments, a FZD-binding agent comprises, consists essentially of, or consists of, the antibody 18R5.

In certain embodiments, a FZD-binding agent (e.g., antibody) competes for specific binding to one or more human FZD proteins with an antibody that comprises a heavy chain variable region comprising SEQ ID NO:3 and a light chain variable region comprising SEQ ID NO:4. In certain embodiments, a FZD-binding agent (e.g., antibody) competes for specific binding to one or more human FZD proteins with an antibody that comprises a heavy chain comprising SEQ ID NO:1 (with or without the signal sequence) and a light chain variable region comprising SEQ ID NO:2 (with or without the signal sequence). In certain embodiments, a FZD-binding agent (e.g., antibody) competes for specific binding to one or more human FZD proteins with an antibody that comprises a heavy chain comprising SEQ ID NO:60 and a light chain variable region comprising SEQ ID NO:61. In certain embodiments, a FZD-binding agent competes with antibody 18R5 for specific binding to one or more human FZD proteins. In some embodiments, a FZD-binding agent or antibody competes for specific binding to one or more human FZD proteins in an in vitro competitive binding assay.

In certain embodiments, a FZD-binding agent (e.g., an antibody) binds the same epitope, or essentially the same epitope, on one or more human FZD proteins as an antibody of the invention. In another embodiment, a FZD-binding agent is an antibody that binds an epitope on one or more human FZD proteins that overlaps with the epitope on a FZD protein bound by an antibody of the invention. In certain embodiments, a FZD-binding agent (e.g., an antibody) binds the same epitope, or essentially the same epitope on one or more FZD proteins as antibody 18R5. In another embodiment, the FZD-binding agent is an antibody that binds an epitope on one or more human FZD proteins that overlaps with the epitope on a FZD protein bound by antibody 18R5.

In certain embodiments, the Wnt pathway inhibitors are agents that bind one or more human Wnt proteins. These agents are referred to herein as “Wnt-binding agents”. In certain embodiments, the agents specifically bind one, two, three, four, five, six, seven, eight, nine, ten, or more Wnt proteins. In some embodiments, the Wnt-binding agents bind one or more human Wnt proteins selected from the group consisting of Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and Wnt16. In certain embodiments, a Wnt-binding agent binds one or more (or two or more, three or more, four or more, five or more, etc.) Wnt proteins selected from the group consisting of Wnt1, Wnt2, Wnt2b, Wnt3, Wnt3a, Wnt7a, Wnt7b, Wnt8a, Wnt8b, Wnt10a, and Wnt10b. In certain embodiments, the one or more (or two or more, three or more, four or more, five or more, etc.) Wnt proteins are selected from the group consisting of Wnt1, Wnt2, Wnt2b, Wnt3, Wnt3a, Wnt8a, Wnt8b, Wnt10a, and Wnt10b.

In certain embodiments, the Wnt-binding agent is a Wnt antagonist. In certain embodiments, the Wnt-binding agent is a Wnt pathway antagonist. In certain embodiments, the Wnt-binding agent inhibits Wnt signaling. In some embodiments, the Wnt-binding agent inhibits canonical Wnt signaling.

In some embodiments, the Wnt-binding agent is an antibody. In some embodiments, the Wnt-binding agent is a polypeptide. In certain embodiments, the Wnt-binding agent is an antibody or a polypeptide comprising an antigen-binding site. In certain embodiments, an antigen-binding site of a Wnt-binding antibody or polypeptide described herein is capable of binding (or binds) one, two, three, four, five, or more human Wnt proteins. In certain embodiments, an antigen-binding site of the Wnt-binding antibody or polypeptide is capable of specifically binding one, two, three, four, or five human Wnt proteins selected from the group consisting of Wnt1, Wnt2, Wnt2b, Wnt3, Wnt3a, Wnt7a, Wnt7b, Wnt8a, Wnt8b, Wnt10a, and Wnt10b. Non-limiting examples of Wnt-binding agents can be found in International Publication WO 2011/088127, which is incorporated by reference herein in its entirety.

In certain embodiments, the Wnt-binding agent binds to the C-terminal cysteine rich domain of one or more human Wnt proteins. In certain embodiments, the Wnt-binding agent binds a domain within the one or more Wnt proteins to which the agent or antibody binds that is selected from the group consisting of: SEQ ID NO:32 (Wnt1), SEQ ID NO:33 (Wnt2), SEQ ID NO:34 (Wnt2b), SEQ ID NO:35 (Wnt3), SEQ ID NO:36 (Wnt3a), SEQ ID NO:37 (Wnt7a), SEQ ID NO:38 (Wnt7b), SEQ ID NO:39 (Wnt8a), SEQ ID NO:40 (Wnt8b), SEQ ID NO:41 (Wnt10a), and SEQ ID NO:42 (Wnt10b).

In certain embodiments, the Wnt-binding agent binds one or more (e.g., two or more, three or more, or four or more) Wnt proteins with a K_{D} of about 1 M or less, about 100 nM or less, about 40 nM or less, about 20 nM or less, or about 10 nM or less. For example, in certain embodiments, a Wnt-binding agent described herein that binds more than one Wnt protein, binds those Wnt proteins with a K_{D} of about 100 nM or less, about 20 nM or less, or about 10 nM or less. In certain embodiments, the Wnt-binding agent binds each of one or more (e.g., 1, 2, 3, 4, or 5) Wnt proteins with a K_{D} of about 40 nM or less, wherein the Wnt proteins are selected from the group consisting of: Wnt1, Wnt2, Wnt2b, Wnt3, Wnt3a, Wnt7a, Wnt7b, Wnt8a, Wnt8b, Wnt10a, and Wnt10b. In some embodiments, the K_{D} of the binding agent (e.g., an antibody) to a Wnt protein is the K_{D} determined using a Wnt fusion protein comprising at least a portion of the Wnt C-terminal cysteine rich domain immobilized on a Biacore chip.

In certain embodiments, the Wnt-binding agent binds one or more (for example, two or more, three or more, or four or more) human Wnt proteins with an EC₅₀ of about 1 μM or less, about 100 nM or less, about 40 nM or less, about 20 nM or less, about 10 nM or less, or about 1 nM or less. In certain embodiments, a Wnt-binding agent binds to more than one Wnt with an EC₅₀ of about 40 nM or less, about 20 nM or less, or about 10 nM or less. In certain embodiments, the Wnt-binding agent has an EC₅₀ of about 20 nM or less with respect to one or more (e.g., 1, 2, 3, 4, or 5) of Wnt proteins Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and/or Wnt16. In certain embodiments, the Wnt-binding agent has an EC₅₀ of about 10 nM or less with respect to one or more (e.g., 1, 2, 3, 4, or 5) of the following Wnt proteins Wnt1, Wnt2, Wnt2b, Wnt3, Wnt3a, Wnt8a, Wnt8b, Wnt10a, and/or Wnt10b.

In certain embodiments, the Wnt pathway inhibitor is a Wnt-binding agent which is an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In certain embodiments, the antibody is an IgG1 antibody. In certain embodiments, the antibody is an IgG2 antibody. In certain embodiments, the antibody is an antibody fragment comprising an antigen-binding site. In some embodiments, the antibody is monovalent, monospecific, bivalent, bispecific, or multispecific. In some embodiments, the antibody is conjugated to a cytotoxic moiety. In some embodiments, the antibody is isolated. In some embodiments, the antibody is substantially pure.

The Wnt-binding agents (e.g., antibodies) of the present invention can be assayed for specific binding by any method known in the art. The immunoassays which can be used include, but are not limited to, competitive and non-competitive assay systems using techniques such as Biacore analysis, FACS analysis, immunofluorescence, immunocytochemistry, Western blots, radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, and protein A immunoassays. Such assays are routine and well-known in the art (see, e.g., Ausubel et al., Editors, 1994-present, Current Protocols in Molecular Biology, John Wiley & Sons, Inc., New York, N.Y.).

For example, the specific binding of an antibody to a human Wnt protein may be determined using ELISA. An ELISA assay comprises preparing antigen, coating wells of a 96 well microtiter plate with antigen, adding the Wnt-binding agent (e.g., an antibody) conjugated to a detectable compound such as an enzymatic substrate (e.g. horseradish peroxidase or alkaline phosphatase) to the well, incubating for a period of time and detecting the presence of the Wnt-binding agent bound to the antigen. In some embodiments, the Wnt-binding antibody or agent is not conjugated to a detectable compound, but instead a second conjugated antibody that recognizes the Wnt-binding antibody or agent is added to the well. In some embodiments, instead of coating the well with the antigen, the Wnt-binding antibody or agent can be coated to the well and a second antibody conjugated to a detectable compound can be added following the addition of the antigen to the coated well. One of skill in the art would be knowledgeable as to the parameters that can be modified to increase the signal detected as well as other variations of ELISAs that may be used.

In another example, the specific binding of an antibody to a human Wnt protein may be determined using FACS. A FACS screening assay may comprise generating a cDNA construct that expresses an antigen as a fusion protein, transfecting the construct into cells, expressing the antigen on the surface of the cells, mixing the Wnt-binding antibody with the transfected cells, and incubating for a period of time. The cells bound by the Wnt-binding antibody may be identified by using a secondary antibody conjugated to a detectable compound (e.g., PE-conjugated anti-Fc antibody) and a flow cytometer. One of skill in the art would be knowledgeable as to the parameters that can be modified to optimize the signal detected as well as other variations of FACS that may enhance screening (e.g., screening for blocking antibodies).

The binding affinity of a Wnt-binding agent to an antigen (e.g., a Wnt protein) and the off-rate of an antibody-antigen interaction can be determined by competitive binding assays such as those described above for FZD-binding agents.

In certain embodiments, the Wnt-binding agent is a soluble receptor. In certain embodiments, the Wnt-binding agent comprises the extracellular domain of a FZD receptor protein. In some embodiments, the Wnt-binding agent comprises a Fri domain of a FZD protein. In some embodiments, soluble receptors comprising a FZD Fri domain can demonstrate altered biological activity (e.g., increased protein half-life) compared to soluble receptors comprising the entire FZD ECD. Protein half-life can be further increased by covalent modification with polyethylene glycol (PEG) or polyethylene oxide (PEO). In certain embodiments, the FZD protein is a human FZD protein. In certain embodiments, the human FZD protein is FZD1, FZD2, FZD3, FZD4, FZD5, FZD6, FZD7, FZD8, FZD9, or FZD10. Non-limiting examples of soluble FZD receptors can be found in U.S. Pat. Nos. 7,723,477 and 7,947,277; and International Publication WO 2011/088123, each of which is incorporated by reference herein in its entirety.

The predicted Fri domains for each of the human FZD1-10 proteins are provided as SEQ ID NOs:11-20. The predicted minimal Fri domains for each of the human FZD1-10 proteins are provided as SEQ ID NOs:48-57. Those of skill in the art may differ in their understanding of the exact amino acids corresponding to the various Fri domains. Thus, the N-terminus and/or C-terminus of the domains outlined above and herein may extend or be shortened by 1, 2, 3, 4, 5, 6, 7, 8, 9, or even 10 amino acids.

In certain embodiments, the Wnt-binding agent comprises a Fri domain of a human FZD protein, or a fragment or variant of the Fri domain that binds one or more human Wnt proteins. In certain embodiments, the human FZD protein is FZD1, FZD2, FZD3, FZD4, FZD5, FZD6, FZD7, FZD8, FZD9, or FZD10. In certain embodiments, the human FZD protein is FZD4. In certain embodiments, the human FZD protein is FZD5. In certain embodiments, the human FZD protein is FZD8. In certain embodiments, the human FZD protein is FZD10. In certain embodiments, the FZD protein is FZD4 and the Wnt-binding agent comprises SEQ ID NO: 14. In certain embodiments, the FZD protein is FZD5 and the Wnt-binding agent comprises SEQ ID NO:15. In certain embodiments, the FZD protein is FZD7 and the Wnt-binding agent comprises SEQ ID NO: 17. In certain embodiments, the FZD protein is FZD8 and the Wnt-binding agent comprises SEQ ID NO:18. In certain embodiments, the FZD protein is FZD100 and the Wnt-binding agent comprises SEQ ID NO:20. In certain embodiments, the FZD protein is FZD8 and the Wnt-binding agent comprises SEQ ID NO:58.

In some embodiments, the Wnt-binding agent comprises a Fri domain comprising the minimal Fri domain of FZD1 (SEQ ID NO:48), the minimal Fri domain of FZD2 (SEQ ID NO:49), the minimal Fri domain of FZD3 (SEQ ID NO:50), the minimal Fri domain of FZD4 (SEQ ID NO:51), the minimal Fri domain of FZD5 (SEQ ID NO:52), the minimal Fri domain of FZD6 (SEQ ID NO:53), the minimal Fri domain of FZD7 (SEQ ID NO:54), the minimal Fri domain of FZD8 (SEQ ID NO:55), the minimal Fri domain of FZD9 (SEQ ID NO:56), or the minimal Fri domain of FZD10 (SEQ ID NO:57). In some embodiments, the Wnt-binding agent comprises a Fri domain comprising the minimal Fri domain of FZD8 (SEQ ID NO:55).

In some embodiments, the Wnt-binding agent comprises a Fri domain consisting essentially of the Fri domain of FZD1, the Fri domain of FZD2, the Fri domain of FZD3, the Fri domain of FZD4, the Fri domain of FZD5, the Fri domain of FZD6, the Fri domain of FZD7, the Fri domain of FZD8, the Fri domain of FZD9, or the Fri domain of FZD10. In some embodiments, the Wnt-binding agent comprises a Fri domain consisting essentially of the Fri domain of FZD8.

In some embodiments, the Wnt-binding agent comprises a sequence selected from the group consisting of: SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, and SEQ ID NO:58. In some embodiments, the Wnt-binding agent comprises a Fri domain consisting essentially of SEQ ID NO: 18. In some embodiments, the Wnt-binding agent comprises a Fri domain consisting essentially of SEQ ID NO:58.

In certain embodiments, the Wnt-binding agent comprises a variant of any one of the aforementioned FZD Fri domain sequences that comprises one or more (e.g., one, two, three, four, five, six, seven, eight, nine, ten, etc.) conservative substitutions and is capable of binding Wnt protein(s).

In certain embodiments, the Wnt-binding agent, such as an agent comprising a Fri domain of a human FZD receptor, further comprises a non-FZD polypeptide. In some embodiments, FZD soluble receptors may include FZD ECD or Fri domains linked to other non-FZD functional and structural polypeptides including, but not limited to, a human Fc region, protein tags (e.g., myc, FLAG, GST), other endogenous proteins or protein fragments, or any other useful protein sequence including any linker region between a FZD ECD or Fri domain and a second polypeptide. In certain embodiments, the non-FZD polypeptide comprises a human Fc region. The Fc region can be obtained from any of the classes of immunoglobulin, IgG, IgA, IgM, IgD and IgE. In some embodiments, the Fc region is a human IgG1 Fc region. In some embodiments, the Fc region is a human IgG2 Fc region. In some embodiments, the Fc region is a wild-type Fc region. In some embodiments, the Fc region is a mutated Fc region. In some embodiments, the Fc region is truncated at the N-terminal end by 1, 2, 3, 4, 5, 6, 7, 8, 9, or 10 amino acids, (e.g., in the hinge domain). In some embodiments, an amino acid in the hinge domain is changed to hinder undesirable disulfide bond formation. In some embodiments, a cysteine is replaced with a serine to hinder undesirable disulfide bond formation. In some embodiments, the Fc region is truncated at the C-terminal end by 1, 2, 3, or more amino acids. In some embodiments, the Fc region is truncated at the C-terminal end by 1 amino acid. In certain embodiments, the non-FZD polypeptide comprises or consists essentially of SEQ ID NO:21, SEQ ID NO:22, SEQ ID NO:23, SEQ ID NO:24, or SEQ ID NO:59. In certain embodiments, the non-FZD polypeptide consists essentially of SEQ ID NO:22 or SEQ ID NO:23.

In certain embodiments, a Wnt-binding agent is a fusion protein comprising at least a minimal Fri domain of a FZD receptor and a Fc region. As used herein, a “fusion protein” is a hybrid protein expressed by a nucleic acid molecule comprising nucleotide sequences of at least two genes. In some embodiments, the C-terminus of the first polypeptide is linked to the N-terminus of the immunoglobulin Fc region. In some embodiments, the first polypeptide (e.g., a FZD Fri domain) is directly linked to the Fc region (i.e. without an intervening peptide linker). In some embodiments, the first polypeptide is linked to the Fc region via a peptide linker.

As used herein, the term “linker” refers to a linker inserted between a first polypeptide (e.g., a FZD component) and a second polypeptide (e.g., a Fc region). In some embodiments, the linker is a peptide linker. Linkers should not adversely affect the expression, secretion, or bioactivity of the polypeptide. Linkers should not be antigenic and should not elicit an immune response. Suitable linkers are known to those of skill in the art and often include mixtures of glycine and serine residues and often include amino acids that are sterically unhindered. Other amino acids that can be incorporated into useful linkers include threonine and alanine residues. Linkers can range in length, for example from 1-50 amino acids in length, 1-22 amino acids in length, 1-10 amino acids in length, 1-5 amino acids in length, or 1-3 amino acids in length. Linkers may include, but are not limited to, SerGly, GGSG, GSGS, GGGS, S(GGS)n where n is 1-7, GRA, poly(Gly), poly(Ala), ESGGGGVT (SEQ ID NO:43), LESGGGGVT (SEQ ID NO:44), GRAQVT (SEQ ID NO:45), WRAQVT (SEQ ID NO:46), and ARGRAQVT (SEQ ID NO:47). As used herein, a linker is an intervening peptide sequence that does not include amino acid residues from either the C-terminus of the first polypeptide (e.g., a FZD Fri domain) or the N-terminus of the second polypeptide (e.g., the Fc region).

In some embodiments, the Wnt-binding agent comprises a FZD Fri domain, a Fc region and a linker connecting the FZD Fri domain to the Fc region. In some embodiments, the FZD Fri domain comprises SEQ ID NO:18, SEQ ID NO:55, or SEQ ID NO:58. In some embodiments, the linker comprises ESGGGGVT (SEQ ID NO:43) or LESGGGGVT (SEQ ID NO:44).

In some embodiments, the Wnt-binding agent comprises a first polypeptide comprising SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, or SEQ ID NO:58; and a second polypeptide comprising SEQ ID NO:21, SEQ ID NO:22, SEQ ID NO:23, SEQ ID NO:24, or SEQ ID NO:59, wherein the first polypeptide is directly linked to the second polypeptide. In some embodiments, the Wnt-binding agent comprises a first polypeptide comprising SEQ ID NO:18, and a second polypeptide comprising SEQ ID NO:21, SEQ ID NO:22, SEQ ID NO:23, SEQ ID NO:24, or SEQ ID NO:59. In some embodiments, the Wnt-binding agent comprises a first polypeptide consisting essentially of SEQ ID NO:18, and a second polypeptide consisting essentially of SEQ ID NO:22 or SEQ ID NO:23. In some embodiments, the Wnt-binding agent comprises a first polypeptide comprising SEQ ID NO:55, and a second polypeptide comprising SEQ ID NO:21, SEQ ID NO:22, SEQ ID NO:23, SEQ ID NO:24, or SEQ ID NO:59. In some embodiments, the Wnt-binding agent comprises a first polypeptide comprising SEQ ID NO:58, and a second polypeptide comprising SEQ ID NO:21, SEQ ID NO:22, SEQ ID NO:23, SEQ ID NO:24, or SEQ ID NO:59. In some embodiments, the Wnt-binding agent comprises a first polypeptide consisting essentially of SEQ ID NO:58, and a second polypeptide consisting essentially of SEQ ID NO:22, SEQ ID NO:23, or SEQ ID NO:59.

In some embodiments, the Wnt-binding agent comprises a first polypeptide comprising SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, or SEQ ID NO:58; and a second polypeptide comprising SEQ ID NO:21, SEQ ID NO:22, SEQ ID NO:23, SEQ ID NO:24, or SEQ ID NO:59, wherein the first polypeptide is connected to the second polypeptide by a linker. In some embodiments, the Wnt-binding agent comprises a first polypeptide comprising SEQ ID NO:18, and a second polypeptide comprising SEQ ID NO:21, SEQ ID NO:22, SEQ ID NO:23, SEQ ID NO:24, or SEQ ID NO:59. In some embodiments, the Wnt-binding agent comprises a first polypeptide consisting essentially of SEQ ID NO:18, and a second polypeptide consisting essentially of SEQ ID NO:22 or SEQ ID NO:23. In some embodiments, the Wnt-binding agent comprises a first polypeptide comprising SEQ ID NO:55, and a second polypeptide comprising SEQ ID NO:21, SEQ ID NO:22, SEQ ID NO:23, SEQ ID NO:24, or SEQ ID NO:59. In some embodiments, the Wnt-binding agent comprises a first polypeptide comprising SEQ ID NO:58, and a second polypeptide comprising SEQ ID NO:21, SEQ ID NO:22, SEQ ID NO:23, SEQ ID NO:24, or SEQ ID NO:59. In some embodiments, the Wnt-binding agent comprises a first polypeptide consisting essentially of SEQ ID NO:58, and a second polypeptide consisting essentially of SEQ ID NO:22, SEQ ID NO:23, or SEQ ID NO:59.

In some embodiments, the Wnt-binding agent comprises a first polypeptide that is at least 95% identical to SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, or SEQ ID NO:58; and a second polypeptide comprising SEQ ID NO:21, SEQ ID NO:22, SEQ ID NO:23, SEQ ID NO:24, or SEQ ID NO:59, wherein the first polypeptide is directly linked to the second polypeptide. In some embodiments, the Wnt-binding agent comprises a first polypeptide that is at least 95% identical to SEQ ID NO: 18, and a second polypeptide comprising SEQ ID NO:21, SEQ ID NO:22, SEQ ID NO:23, SEQ ID NO:24, or SEQ ID NO:59. In some embodiments, the Wnt-binding agent comprises a first polypeptide that is at least 95% identical to SEQ ID NO:55, and a second polypeptide comprising SEQ ID NO:21, SEQ ID NO:22, SEQ ID NO:23, SEQ ID NO:24, or SEQ ID NO:59. In some embodiments, the Wnt-binding agent comprises a first polypeptide that is at least 95% identical to SEQ ID NO:58, and a second polypeptide comprising SEQ ID NO:21, SEQ ID NO:22, SEQ ID NO:23, SEQ ID NO:24, or SEQ ID NO:59.

In some embodiments, the Wnt-binding agent comprises a first polypeptide that is at least 95% identical to SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, or SEQ ID NO:58; and a second polypeptide comprising SEQ ID NO:21, SEQ ID NO:22, SEQ ID NO:23, SEQ ID NO:24, or SEQ ID NO:59, wherein the first polypeptide is connected to the second polypeptide by a linker. In some embodiments, the Wnt-binding agent comprises a first polypeptide that is at least 95% identical to SEQ ID NO:18, and a second polypeptide comprising SEQ ID NO:21, SEQ ID NO:22, SEQ ID NO:23, SEQ ID NO:24, or SEQ ID NO:59. In some embodiments, the Wnt-binding agent comprises a first polypeptide that is at least 95% identical to SEQ ID NO:55, and a second polypeptide comprising SEQ ID NO:21, SEQ ID NO:22, SEQ ID NO:23, SEQ ID NO:24, or SEQ ID NO:59. In some embodiments, the Wnt-binding agent comprises a first polypeptide that is at least 95% identical to SEQ ID NO:58, and a second polypeptide comprising SEQ ID NO:21, SEQ ID NO:22, SEQ ID NO:23, SEQ ID NO:24, or SEQ ID NO:59.

FZD proteins contain a signal sequence that directs the transport of the proteins. Signal sequences (also referred to as signal peptides or leader sequences) are located at the N-terminus of nascent polypeptides. They target the polypeptide to the endoplasmic reticulum and the proteins are sorted to their destinations, for example, to the inner space of an organelle, to an interior membrane, to the cell outer membrane, or to the cell exterior via secretion. Most signal sequences are cleaved from the protein by a signal peptidase after the proteins are transported to the endoplasmic reticulum. The cleavage of the signal sequence from the polypeptide usually occurs at a specific site in the amino acid sequence and is dependent upon amino acid residues within the signal sequence. Although there is usually one specific cleavage site, more than one cleavage site may be recognized and/or used by a signal peptidase resulting in a non-homogenous N-terminus of the polypeptide. For example, the use of different cleavage sites within a signal sequence can result in a polypeptide expressed with different N-terminal amino acids. Accordingly, in some embodiments, the polypeptides as described herein may comprise a mixture of polypeptides with different N-termini. In some embodiments, the N-termini differ in length by 1, 2, 3, 4, 5, 6, 7, 8, 9, 10, or more amino acids. In some embodiments, the N-termini differ in length by 1, 2, 3, 4, or 5 amino acids. In some embodiments, the polypeptide is substantially homogeneous, i.e., the polypeptides have the same N-terminus. In some embodiments, the signal sequence of the polypeptide comprises one or more (e.g., one, two, three, four, five, six, seven, eight, nine, ten, etc.) amino acid substitutions and/or deletions. In some embodiments, the signal sequence of the polypeptide comprises amino acid substitutions and/or deletions that allow one cleavage site to be dominant, thereby resulting in a substantially homogeneous polypeptide with one N-terminus.

In some embodiments, the Wnt-binding agent comprises an amino acid sequence selected from the group consisting of: SEQ ID NO:25, SEQ ID NO:26, SEQ ID NO:27, SEQ ID NO:28, SEQ ID NO:29, SEQ ID NO:30, and SEQ ID NO:31.

In certain embodiments, the Wnt-binding agent comprises the sequence of SEQ ID NO:25. In certain embodiments, the agent comprises the sequence of SEQ ID NO:25, comprising one or more (e.g., one, two, three, four, five, six, seven, eight, nine, ten, etc.) conservative substitutions. In certain embodiments, the agent comprises a sequence having at least about 90%, about 95%, or about 98% sequence identity with SEQ ID NO:25. In certain embodiments, the variants of SEQ ID NO:25 maintain the ability to bind one or more human Wnt proteins.

In certain embodiments, the Wnt-binding agent comprises the sequence of SEQ ID NO:26. In some embodiments, the Wnt-binding agent is SEQ ID NO:26. In certain alternative embodiments, the agent comprises the sequence of SEQ ID NO:26, comprising one or more (e.g., one, two, three, four, five, six, seven, eight, nine, ten, etc.) conservative substitutions. In certain embodiments, the agent comprises a sequence having at least about 90%, about 95%, or about 98% sequence identity with SEQ ID NO:26. In certain embodiments, the variants of SEQ ID NO:26 maintain the ability to bind one or more human Wnt proteins.

In certain embodiments, the Wnt-binding agent comprises the sequence of SEQ ID NO:27. In some embodiments, the Wnt-binding agent is SEQ ID NO:27. In certain alternative embodiments, the agent comprises the sequence of SEQ ID NO:27, comprising one or more (e.g., one, two, three, four, five, six, seven, eight, nine, ten, etc.) conservative substitutions. In certain embodiments, the agent comprises a sequence having at least about 90%, about 95%, or about 98% sequence identity with SEQ ID NO:27. In certain embodiments, the variants of SEQ ID NO:27 maintain the ability to bind one or more human Wnt proteins.

In certain embodiments, a Wnt-binding agent is a polypeptide comprising an amino acid sequence selected from the group consisting of: SEQ ID NO:25, SEQ ID NO:26, SEQ ID NO:27, SEQ ID NO:28, SEQ ID NO:29, SEQ ID NO:30, and SEQ ID NO:31. In certain embodiments, the polypeptide comprises an amino acid sequence selected from the group consisting of SEQ ID NO:25, SEQ ID NO:26, and SEQ ID NO:27. In some embodiments, a polypeptide consists essentially of an amino acid sequence selected from the group consisting of: SEQ ID NO:25, SEQ ID NO:26, and SEQ ID NO:27. In certain embodiments, the polypeptide comprises the amino acid sequence of SEQ ID NO:25. In some embodiments, the polypeptide comprises the amino acid sequence of SEQ ID NO:26. In certain embodiments, the polypeptide comprises the amino acid sequence of SEQ ID NO:27. In certain embodiments, the polypeptide comprises the amino acid sequence of SEQ ID NO:28. In certain embodiments, the polypeptide comprises the amino acid sequence of SEQ ID NO:29. In certain embodiments, the polypeptide comprises the amino acid sequence of SEQ ID NO:30. In certain embodiments, the polypeptide comprises the amino acid sequence of SEQ ID NO:31.

In some embodiments, the polypeptide is a substantially purified polypeptide comprising an amino acid sequence selected from the group consisting of SEQ ID NO:25, SEQ ID NO:26, and SEQ ID NO:27. In some embodiments, the polypeptide is a substantially purified polypeptide comprising SEQ ID NO:27. In certain embodiments, the substantially purified polypeptide consists of at least 90% of a polypeptide that has an N-terminal sequence of ASA. In some embodiments, the nascent polypeptide comprises a signal sequence that results in a substantially homogeneous polypeptide product with one N-terminal sequence.

In certain embodiments, a Wnt-binding agent comprises a Fc region of an immunoglobulin. Those skilled in the art will appreciate that some of the binding agents of this invention will comprise fusion proteins in which at least a portion of the Fc region has been deleted or otherwise altered so as to provide desired biochemical characteristics, such as increased cancer cell localization, increased tumor penetration, reduced serum half-life, or increased serum half-life, when compared with a fusion protein of approximately the same immunogenicity comprising a native or unaltered constant region. Modifications to the Fc region may include additions, deletions, or substitutions of one or more amino acids in one or more domains. The modified fusion proteins disclosed herein may comprise alterations or modifications to one or more of the two heavy chain constant domains (CH2 or CH3) or to the hinge region. In other embodiments, the entire CH2 domain may be removed (ΔCH2 constructs). In some embodiments, the omitted constant region domain is replaced by a short amino acid spacer (e.g., 10 aa residues) that provides some of the molecular flexibility typically imparted by the absent constant region domain.

In some embodiments, the modified fusion proteins are engineered to link the CH3 domain directly to the hinge region. In other embodiments, a peptide spacer is inserted between the hinge region and the modified CH2 and/or CH3 domains. For example, constructs may be expressed wherein the CH2 domain has been deleted and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the fusion protein.

In some embodiments, the modified fusion proteins may have only a partial deletion of a constant domain or substitution of a few or even a single amino acid. For example, the mutation of a single amino acid in selected areas of the CH2 domain may be enough to substantially reduce Fc binding and thereby increase cancer cell localization and/or tumor penetration. Similarly, it may be desirable to simply delete that part of one or more constant region domains that control a specific effector function (e.g., complement C1q binding). Such partial deletions of the constant regions may improve selected characteristics of the binding agent (e.g., serum half-life) while leaving other desirable functions associated with the subject constant region domain intact. Moreover, as alluded to above, the constant regions of the disclosed fusion proteins may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified fusion protein. In certain embodiments, the modified fusion proteins comprise the addition of one or more amino acids to the constant region to enhance desirable characteristics such as decreasing or increasing effector function, or provide for more cytotoxin or carbohydrate attachment sites.

It is known in the art that the constant region mediates several effector functions. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region of an immunoglobulin can bind to a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells, release of inflammatory mediators, placental transfer, and control of immunoglobulin production.

In some embodiments, the modified fusion proteins provide for altered effector functions that, in turn, affect the biological profile of the administered agent. For example, in some embodiments, the deletion or inactivation (through point mutations or other means) of a constant region domain may reduce Fc receptor binding of the circulating modified agent, thereby increasing cancer cell localization and/or tumor penetration. In other embodiments, the constant region modifications increase or reduce the serum half-life of the agent. In some embodiments, the constant region is modified to eliminate disulfide linkages or oligosaccharide moieties.

In certain embodiments, a modified fusion protein does not have one or more effector functions normally associated with an Fc region. In some embodiments, the agent has no antibody-dependent cell-mediated cytotoxicity (ADCC) activity, and/or no complement-dependent cytotoxicity (CDC) activity. In certain embodiments, the agent does not bind to the Fc receptor and/or complement factors. In certain embodiments, the agent has no effector function.

In some embodiments, the Wnt-binding agent (e.g., a soluble receptor) described herein is modified to reduce immunogenicity. In general, immune responses against completely normal human proteins are rare when these proteins are used as therapeutics. However, although many fusion proteins comprise polypeptides sequences that are the same as the sequences found in nature, several therapeutic fusion proteins have been shown to be immunogenic in mammals. In some studies, a fusion protein comprising a linker has been found to be more immunogenic than a fusion protein that does not contain a linker. Accordingly, in some embodiments, the polypeptides of the invention are analyzed by computation methods to predict immunogenicity. In some embodiments, the polypeptides are analyzed for the presence of T-cell and/or B-cell epitopes. If any T-cell or B-cell epitopes are identified and/or predicted, modifications to these regions (e.g., amino acid substitutions) may be made to disrupt or destroy the epitopes. Various algorithms and software that can be used to predict T-cell and/or B-cell epitopes are known in the art. For example, the software programs SYFPEITHI, HLA Bind, PEPVAC, RANKPEP, DiscoTope, ElliPro, and Antibody Epitope Prediction are all publicly available.

In some embodiments, a cell producing any of the Wnt-binding agents (e.g., soluble receptors) or polypeptides described herein is provided. In some embodiments, a composition comprising any of the Wnt-binding agents (e.g., soluble receptors) or polypeptides described herein is provided. In some embodiments, the composition comprises a polypeptide wherein at least 80%, 90%, 95%, 97%, 98%, or 99% of the polypeptide has an N-terminal sequence of ASA. In some embodiments, the composition comprises a polypeptide wherein 100% of the polypeptide has an N-terminal sequence of ASA. In some embodiments, the composition comprises a polypeptide wherein at least 80% of the polypeptide has an N-terminal sequence of ASA. In some embodiments, the composition comprises a polypeptide wherein at least 90% of the polypeptide has an N-terminal sequence of ASA. In some embodiments, the composition comprises a polypeptide wherein at least 95% of the polypeptide has an N-terminal sequence of ASA.

The polypeptides described herein can be recombinant polypeptides, natural polypeptides, or synthetic polypeptides. It will be recognized in the art that some amino acid sequences of the invention can be varied without significant effect on the structure or function of the protein. If such differences in sequence are contemplated, it should be remembered that there will be critical areas on the protein which determine activity. Thus, the invention further includes variations of the polypeptides which show substantial activity or which include regions of FZD proteins, such as the protein portions discussed herein. Such mutants include deletions, insertions, inversions, repeats, and type substitutions.

Of course, the number of amino acid substitutions a skilled artisan would make depends on many factors, including those described above. In certain embodiments, the number of substitutions for any given soluble receptor polypeptide will not be more than 50, 40, 30, 25, 20, 15, 10, 5 or 3.

Fragments or portions of the polypeptides of the present invention can be employed for producing the corresponding full-length polypeptide by peptide synthesis; therefore, the fragments can be employed as intermediates for producing the full-length polypeptides. These fragments or portion of the polypeptides can also be referred to as “protein fragments” or “polypeptide fragments”.

A protein fragment of this invention is a portion or all of a protein which is capable of binding to one or more human Wnt proteins or one or more human FZD proteins. In some embodiments, the fragment has a high affinity for one or more human Wnt proteins. In some embodiments, the fragment has a high affinity for one or more human FZD proteins. Some fragments of Wnt-binding agents described herein are protein fragments comprising at least part of the extracellular portion of a FZD protein linked to at least part of a constant region of an immunoglobulin (e.g., a Fc region). The binding affinity of the protein fragment can be in the range of about 10⁻¹¹ to 10⁻¹² M, although the affinity can vary considerably with fragments of different sizes, ranging from 10⁻⁷ to 10⁻¹³ M. In some embodiments, the fragment is about 100 to about 200 amino acids in length and comprises a binding domain linked to at least part of a constant region of an immunoglobulin.

In some embodiments, the Wnt pathway inhibitors are polyclonal antibodies. Polyclonal antibodies can be prepared by any known method. In some embodiments, polyclonal antibodies are raised by immunizing an animal (e.g., a rabbit, rat, mouse, goat, donkey) by multiple subcutaneous or intraperitoneal injections of the relevant antigen (e.g., a purified peptide fragment, full-length recombinant protein, or fusion protein). The antigen can be optionally conjugated to a carrier such as keyhole limpet hemocyanin (KLH) or serum albumin. The antigen (with or without a carrier protein) is diluted in sterile saline and usually combined with an adjuvant (e.g., Complete or Incomplete Freund's Adjuvant) to form a stable emulsion. After a sufficient period of time, polyclonal antibodies are recovered from blood and/or ascites of the immunized animal. The polyclonal antibodies can be purified from serum or ascites according to standard methods in the art including, but not limited to, affinity chromatography, ion-exchange chromatography, gel electrophoresis, and dialysis.

In some embodiments, the Wnt pathway inhibitors are monoclonal antibodies. Monoclonal antibodies can be prepared using hybridoma methods known to one of skill in the art (see e.g., Kohler and Milstein, 1975, Nature, 256:495-497). In some embodiments, using the hybridoma method, a mouse, hamster, or other appropriate host animal, is immunized as described above to elicit from lymphocytes the production of antibodies that will specifically bind the immunizing antigen. In some embodiments, lymphocytes can be immunized in vitro. In some embodiments, the immunizing antigen can be a human protein or a portion thereof. In some embodiments, the immunizing antigen can be a mouse protein or a portion thereof.

Following immunization, lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen may be identified by a variety of methods including, but not limited to, immunoprecipitation, immunoblotting, and in vitro binding assay (e.g., flow cytometry, FACS, ELISA, and radioimmunoassay). The hybridomas can be propagated either in in vitro culture using standard methods (J. W. Goding, 1996, Monoclonal Antibodies: Principles and Practice, 3rd Edition, Academic Press, San Diego, Calif.) or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In certain embodiments, monoclonal antibodies can be made using recombinant DNA techniques as known to one skilled in the art (see e.g., U.S. Pat. No. 4,816,567). The polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using conventional techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins. In other embodiments, recombinant monoclonal antibodies, or fragments thereof, can be isolated from phage display libraries (see e.g., McCafferty et al., 1990, Nature, 348:552-554; Clackson et al., 1991, Nature, 352:624-628; and Marks et al., 1991, J. Mol. Biol., 222:581-597).

The polynucleotide(s) encoding a monoclonal antibody can further be modified in a number of different manners using recombinant DNA technology to generate alternative antibodies. In some embodiments, the constant domains of the light and heavy chains of, for example, a mouse monoclonal antibody can be substituted for those regions of, for example, a human antibody to generate a chimeric antibody, or for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

In some embodiments, the Wnt pathway inhibitor is a humanized antibody. Typically, humanized antibodies are human immunoglobulins in which residues from the CDRs are replaced by residues from a CDR of a non-human species (e.g., mouse, rat, rabbit, hamster, etc.) that have the desired specificity, affinity, and/or binding capability using methods known to one skilled in the art. In some embodiments, the Fv framework region residues of a human immunoglobulin are replaced with the corresponding residues in an antibody from a non-human species that has the desired specificity, affinity, and/or binding capability. In some embodiments, the humanized antibody can be further modified by the substitution of additional residues either in the Fv framework region and/or within the replaced non-human residues to refine and optimize antibody specificity, affinity, and/or capability. In general, the humanized antibody will comprise substantially all of at least one, and typically two, variable domain regions containing all, or substantially all, of the CDRs that correspond to the non-human immunoglobulin whereas all, or substantially all, of the framework regions are those of a human immunoglobulin consensus sequence. In some embodiments, the humanized antibody can also comprise at least a portion of an immunoglobulin constant region or domain (Fc), typically that of a human immunoglobulin. In certain embodiments, such humanized antibodies are used therapeutically because they may reduce antigenicity and HAMA (human anti-mouse antibody) responses when administered to a human subject. One skilled in the art would be able to obtain a functional humanized antibody with reduced immunogenicity following known techniques (see e.g., U.S. Pat. Nos. 5,225,539; 5,585,089; 5,693,761; and 5,693,762).

In certain embodiments, the Wnt pathway inhibitor is a human antibody. Human antibodies can be directly prepared using various techniques known in the art. In some embodiments, immortalized human B lymphocytes immunized in vitro or isolated from an immunized individual that produces an antibody directed against a target antigen can be generated (see, e.g., Cole et al., 1985, Monoclonal Antibodies and Cancer Therapy, Alan R. Liss, p. 77; Boemer et al., 1991, J. Immunol., 147:86-95; and U.S. Pat. Nos. 5,750,373; 5,567,610; and 5,229,275). In some embodiments, the human antibody can be selected from a phage library, where that phage library expresses human antibodies (Vaughan et al., 1996, Nature Biotechnology, 14:309-314; Sheets et al., 1998, PNAS, 95:6157-6162; Hoogenboom and Winter, 1991, J. Mol. Biol., 227:381; Marks et al., 1991, J. Mol. Biol., 222:581). Alternatively, phage display technology can be used to produce human antibodies and antibody fragments in vitro, from immunoglobulin variable domain gene repertoires from unimmunized donors. Techniques for the generation and use of antibody phage libraries are described in U.S. Pat. Nos. 5,969,108; 6,172,197; 5,885,793; 6,521,404; 6,544,731; 6,555,313; 6,582,915; 6,593,081; 6,300,064; 6,653,068; 6,706,484; and 7,264,963; and Rothe et al., 2008, J. Mol. Bio., 376:1182-1200. Affinity maturation strategies including, but not limited to, chain shuffling (Marks et al., 1992, Bio/Technology, 10:779-783) and site-directed mutagenesis, are known in the art and may be employed to generate high affinity human antibodies.

In some embodiments, human antibodies can be made in transgenic mice that contain human immunoglobulin loci. These mice are capable, upon immunization, of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

This invention also encompasses bispecific antibodies that specifically recognize at least one human FZD protein or at least one Wnt protein. Bispecific antibodies are capable of specifically recognizing and binding at least two different epitopes. The different epitopes can either be within the same molecule (e.g., two different epitopes on human FZD5) or on different molecules (e.g., one epitope on FZD5 and a different epitope on a second protein). In some embodiments, the bispecific antibodies are monoclonal human or humanized antibodies. In some embodiments, the antibodies can specifically recognize and bind a first antigen target, (e.g., a FZD protein) as well as a second antigen target, such as an effector molecule on a leukocyte (e.g., CD2, CD3, CD28, CD80 or CD86) or a Fc receptor (e.g., CD64, CD32, or CD16) so as to focus cellular defense mechanisms to the cell expressing the first antigen target. In some embodiments, the antibodies can be used to direct cytotoxic agents to cells which express a particular target antigen. These antibodies possess an antigen-binding arm and an arm which binds a cytotoxic agent or a radionuclide chelator, such as EOTUBE, DPTA, DOTA, or TETA.

Techniques for making bispecific antibodies are known by those skilled in the art, see for example, Millstein et al., 1983, Nature, 305:537-539; Brennan et al., 1985, Science, 229:81; Suresh et al., 1986, Methods in Enzymol., 121:120; Traunecker et al., 1991, EMBO J., 10:3655-3659; Shalaby et al., 1992, J. Exp. Med., 175:217-225; Kostelny et al., 1992, J. Immunol., 148:1547-1553; Gruber et al., 1994, J. Immunol., 152:5368; U.S. Pat. No. 5,731,168; and U.S. Patent Publication No. 2011/0123532. Bispecific antibodies can be intact antibodies or antibody fragments. Antibodies with more than two valencies are also contemplated. For example, trispecific antibodies can be prepared (Tutt et al., 1991, J. Immunol., 147:60). Thus, in certain embodiments the antibodies are multispecific.

In certain embodiments, the antibodies (or other polypeptides) described herein may be monospecific. For example, in certain embodiments, each of the one or more antigen-binding sites that an antibody contains is capable of binding (or binds) a homologous epitope on different proteins. In certain embodiments, an antigen-binding site of a monospecific antibody described herein is capable of binding (or binds), for example, FZD5 and FZD7 (i.e., the same epitope is found on both FZD5 and FZD7 proteins).

In certain embodiments, the Wnt pathway inhibitor is an antibody fragment comprising an antigen-binding site. Antibody fragments may have different functions or capabilities than intact antibodies; for example, antibody fragments can have increased tumor penetration. Various techniques are known for the production of antibody fragments including, but not limited to, proteolytic digestion of intact antibodies. In some embodiments, antibody fragments include a F(ab′)2 fragment produced by pepsin digestion of an antibody molecule. In some embodiments, antibody fragments include a Fab fragment generated by reducing the disulfide bridges of an F(ab′)2 fragment. In other embodiments, antibody fragments include a Fab fragment generated by the treatment of the antibody molecule with papain and a reducing agent. In certain embodiments, antibody fragments are produced recombinantly. In some embodiments, antibody fragments include Fv or single chain Fv (scFv) fragments. Fab, Fv, and scFv antibody fragments can be expressed in and secreted from E. coli or other host cells, allowing for the production of large amounts of these fragments. In some embodiments, antibody fragments are isolated from antibody phage libraries as discussed herein. For example, methods can be used for the construction of Fab expression libraries (Huse et al., 1989, Science, 246:1275-1281) to allow rapid and effective identification of monoclonal Fab fragments with the desired specificity for a FZD or Wnt protein or derivatives, fragments, analogs or homologs thereof. In some embodiments, antibody fragments are linear antibody fragments. In certain embodiments, antibody fragments are monospecific or bispecific. In certain embodiments, the Wnt pathway inhibitor is a scFv. Various techniques can be used for the production of single-chain antibodies specific to one or more human FZD proteins or one or more human Wnt proteins (see, e.g., U.S. Pat. No. 4,946,778).

It can further be desirable, especially in the case of antibody fragments, to modify an antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis). In some embodiments, an antibody is modified to decrease its serum half-life.

Heteroconjugate antibodies are also within the scope of the present invention. Heteroconjugate antibodies are composed of two covalently joined antibodies. Such antibodies have, for example, been proposed to target immune cells to unwanted cells (U.S. Pat. No. 4,676,980). It is also contemplated that the heteroconjugate antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

For the purposes of the present invention, it should be appreciated that modified antibodies can comprise any type of variable region that provides for the association of the antibody with the target (i.e., a human FZD protein or a human Wnt protein). In this regard, the variable region may comprise or be derived from any type of mammal that can be induced to mount a humoral response and generate immunoglobulins against the desired tumor-associated antigen. As such, the variable region of the modified antibodies can be, for example, of human, murine, non-human primate (e.g. cynomolgus monkeys, macaques, etc.) or rabbit origin. In some embodiments, both the variable and constant regions of the modified immunoglobulins are human. In other embodiments, the variable regions of compatible antibodies (usually derived from a non-human source) can be engineered or specifically tailored to improve the binding properties or reduce the immunogenicity of the molecule. In this respect, variable regions useful in the present invention can be humanized or otherwise altered through the inclusion of imported amino acid sequences.

In certain embodiments, the variable domains in both the heavy and light chains are altered by at least partial replacement of one or more CDRs and, if necessary, by partial framework region replacement and sequence modification and/or alteration. Although the CDRs may be derived from an antibody of the same class or even subclass as the antibody from which the framework regions are derived, it is envisaged that the CDRs will be derived preferably from an antibody from a different species. It may not be necessary to replace all of the CDRs with all of the CDRs from the donor variable region to transfer the antigen binding capacity of one variable domain to another. Rather, it may only be necessary to transfer those residues that are necessary to maintain the activity of the antigen-binding site.

Alterations to the variable region notwithstanding, those skilled in the art will appreciate that the modified antibodies of this invention will comprise antibodies (e.g., full-length antibodies or immunoreactive fragments thereof) in which at least a fraction of one or more of the constant region domains has been deleted or otherwise altered so as to provide desired biochemical characteristics such as increased tumor localization and/or increased serum half-life when compared with an antibody of approximately the same immunogenicity comprising a native or unaltered constant region. In some embodiments, the constant region of the modified antibodies will comprise a human constant region. Modifications to the constant region compatible with this invention comprise additions, deletions or substitutions of one or more amino acids in one or more domains. The modified antibodies disclosed herein may comprise alterations or modifications to one or more of the three heavy chain constant domains (CH1, CH2 or CH3) and/or to the light chain constant domain (CL). In some embodiments, one or more domains are partially or entirely deleted from the constant regions of the modified antibodies. In some embodiments, the modified antibodies will comprise domain deleted constructs or variants wherein the entire CH2 domain has been removed (ΔCH2 constructs). In some embodiments, the omitted constant region domain is replaced by a short amino acid spacer (e.g., 10 amino acid residues) that provides some of the molecular flexibility typically imparted by the absent constant region.

In some embodiments, the modified antibodies are engineered to fuse the CH3 domain directly to the hinge region of the antibody. In other embodiments, a peptide spacer is inserted between the hinge region and the modified CH2 and/or CH3 domains. For example, constructs may be expressed wherein the CH2 domain has been deleted and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the modified antibodies.

In some embodiments, the modified antibodies may have only a partial deletion of a constant domain or substitution of a few or even a single amino acid. For example, the mutation of a single amino acid in selected areas of the CH2 domain may be enough to substantially reduce Fc binding and thereby increase cancer cell localization and/or tumor penetration. Similarly, it may be desirable to simply delete the part of one or more constant region domains that control a specific effector function (e.g. complement C1q binding). Such partial deletions of the constant regions may improve selected characteristics of the antibody (serum half-life) while leaving other desirable functions associated with the subject constant region domain intact. Moreover, as alluded to above, the constant regions of the disclosed antibodies may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified antibody. In certain embodiments, the modified antibodies comprise the addition of one or more amino acids to the constant region to enhance desirable characteristics such as decreasing or increasing effector function or provide for more cytotoxin or carbohydrate attachment sites.

It is known in the art that the constant region mediates several effector functions. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells, release of inflammatory mediators, placental transfer, and control of immunoglobulin production.

In certain embodiments, the Wnt pathway inhibitors are antibodies that provide for altered effector functions. These altered effector functions may affect the biological profile of the administered antibody. For example, in some embodiments, the deletion or inactivation (through point mutations or other means) of a constant region domain may reduce Fc receptor binding of the circulating modified antibody (e.g., anti-FZD antibody) thereby increasing cancer cell localization and/or tumor penetration. In other embodiments, the constant region modifications increase or reduce the serum half-life of the antibody. In some embodiments, the constant region is modified to eliminate disulfide linkages or oligosaccharide moieties. Modifications to the constant region in accordance with this invention may easily be made using well known biochemical or molecular engineering techniques well within the purview of the skilled artisan.

In certain embodiments, a Wnt pathway inhibitor is an antibody does not have one or more effector functions. For instance, in some embodiments, the antibody has no ADCC activity, and/or no CDC activity. In certain embodiments, the antibody does not bind an Fc receptor, and/or complement factors. In certain embodiments, the antibody has no effector function.

The present invention further embraces variants and equivalents which are substantially homologous to the chimeric, humanized, and human antibodies, or antibody fragments thereof, set forth herein. These can contain, for example, conservative substitution mutations, i.e. the substitution of one or more amino acids by similar amino acids. For example, conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well known in the art and described herein.

Thus, the present invention provides methods for producing an antibody. In some embodiments, the method for producing an antibody comprises using hybridoma techniques. In some embodiments, a method for producing an antibody that binds a human FZD protein is provided. In some embodiments, a method for producing an antibody that binds a human Wnt protein is provided. In some embodiments, the method of generating an antibody comprises screening a human phage library. In some embodiments, the antibody is identified using a membrane-bound heterodimeric molecule comprising a single antigen-binding site. In some non-limiting embodiments, the antibody is identified using methods and polypeptides described in International Publication WO 2011/100566, which is incorporated by reference herein in its entirety.

The present invention further provides methods of identifying an antibody that binds at least one FZD protein. In some embodiments, the antibody is identified by screening by FACS for binding to a FZD protein or a portion thereof. In some embodiments, the antibody is identified by screening using ELISA for binding to a FZD protein. In some embodiments, the antibody is identified by screening by FACS for blocking of binding of a FZD protein to a human Wnt protein. In some embodiments, the antibody is identified by screening for inhibition or blocking of Wnt pathway signaling.

The present invention further provides methods of identifying an antibody that binds at least one Wnt protein. In some embodiments, the antibody is identified by screening by FACS for binding to a Wnt protein or a portion thereof. In some embodiments, the antibody is identified by screening using ELISA for binding to a Wnt protein. In some embodiments, the antibody is identified by screening by FACS for blocking of binding of a Wnt protein to a human FZD protein. In some embodiments, the antibody is identified by screening for inhibition or blocking of Wnt pathway signaling.

In some embodiments, a method of generating an antibody to at least one human FZD protein comprises screening an antibody-expressing library for antibodies that bind a human FZD protein. In some embodiments, the antibody-expressing library is a phage library. In some embodiments, the antibody-expressing library is a mammalian cell library. In some embodiments, the screening comprises panning. In some embodiments, antibodies identified in a first screening, are screened again using a different FZD protein thereby identifying an antibody that binds the first FZD protein and a second FZD protein. In some embodiments, the antibody identified in the screening binds the first FZD protein and at least one other FZD protein. In certain embodiments, the at least one other FZD protein is selected from the group consisting of FZD1, FZD2, FZD3, FZD4, FZD5, FZD6, FZD7, FZD8, FZD9, and FZD10. In certain embodiments, the antibody identified in the screening binds FZD1, FZD2, FZD5, FZD7, and FZD8. In some embodiments, the antibody identified in the screening is a FZD antagonist. In some embodiments, the antibody identified by the methods described herein inhibits the Wnt pathway. In some embodiments, the antibody identified in the screening inhibits β-catenin signaling.

In some embodiments, a method of generating an antibody to at least one human Wnt protein comprises screening an antibody-expressing library for antibodies that bind a human Wnt protein. In some embodiments, the antibody-expressing library is a phage library. In some embodiments, the antibody-expressing library is a mammalian cell library. In some embodiments, the screening comprises panning. In some embodiments, antibodies identified in a first screening, are screened again using a different Wnt protein thereby identifying an antibody that binds a first Wnt protein and a second Wnt protein. In some embodiments, the antibody identified in the screening binds a first Wnt protein and at least one other Wnt protein. In certain embodiments, the at least one other FZD protein is selected from the group consisting of Wnt1, Wnt2, Wnt2b, Wnt3, Wnt3a, Wnt7a, Wnt7b, Wnt8a, Wnt8b, Wnt10a, and Wnt10b. In some embodiments, the antibody identified in the screening is a Wnt antagonist. In some embodiments, the antibody identified by the methods described herein inhibits the Wnt pathway. In some embodiments, the antibody identified in the screening inhibits β-catenin signaling.

In certain embodiments, the antibodies described herein are isolated. In certain embodiments, the antibodies described herein are substantially pure.

In some embodiments of the present invention, the Wnt pathway inhibitors are polypeptides. The polypeptides can be recombinant polypeptides, natural polypeptides, or synthetic polypeptides comprising an antibody, or fragment thereof, that bind at least one human FZD protein or at least one Wnt protein. It will be recognized in the art that some amino acid sequences of the invention can be varied without significant effect on the structure or function of the protein. Thus, the invention further includes variations of the polypeptides which show substantial activity or which include regions of an antibody, or fragment thereof, against a human FZD protein or a Wnt protein. In some embodiments, amino acid sequence variations of FZD-binding polypeptides or Wnt-binding polypeptides include deletions, insertions, inversions, repeats, and/or other types of substitutions.

The polypeptides, analogs and variants thereof, can be further modified to contain additional chemical moieties not normally part of the polypeptide. The derivatized moieties can improve the solubility, the biological half-life, and/or absorption of the polypeptide. The moieties can also reduce or eliminate any undesirable side effects of the polypeptides and variants. An overview for chemical moieties can be found in Remington: The Science and Practice of Pharmacy, 21st Edition, 2005, University of the Sciences, Philadelphia, Pa.

The isolated polypeptides described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g., Zoeller et al., 1984, PNAS, 81:5662-5066 and U.S. Pat. No. 4,588,585.

In some embodiments, a DNA sequence encoding a polypeptide of interest may be constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), the polynucleotide sequences encoding a particular polypeptide of interest can be inserted into an expression vector and operatively linked to an expression control sequence appropriate for expression of the protein in a desired host. Proper assembly can be confirmed by nucleotide sequencing, restriction enzyme mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In certain embodiments, recombinant expression vectors are used to amplify and express DNA encoding binding agents (e.g., antibodies or soluble receptors), or fragments thereof, against a human FZD protein or a Wnt protein. For example, recombinant expression vectors can be replicable DNA constructs which have synthetic or cDNA-derived DNA fragments encoding a polypeptide chain of a FZD-binding agent, a Wnt-binding agent, an anti-FZD antibody or fragment thereof, an anti-Wnt antibody or fragment thereof, or a FZD-Fc soluble receptor operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In other embodiments, where recombinant protein is expressed without a leader or transport sequence, it can include an N-terminal methionine residue. This residue can optionally be subsequently cleaved from the expressed recombinant protein to provide a final product.

The choice of an expression control sequence and an expression vector depends upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

Suitable host cells for expression of a FZD-binding or Wnt-binding agent (or a protein to use as an antigen) include prokaryotes, yeast cells, insect cells, or higher eukaryotic cells under the control of appropriate promoters. Prokaryotes include gram-negative or gram-positive organisms, for example E. coli or Bacillus. Higher eukaryotic cells include established cell lines of mammalian origin as described below. Cell-free translation systems may also be employed. Appropriate cloning and expression vectors for use with bacterial, fungal, yeast, and mammalian cellular hosts are described by Pouwels et al. (1985, Cloning Vectors: A Laboratory Manual, Elsevier, New York, N.Y.). Additional information regarding methods of protein production, including antibody production, can be found, e.g., in U.S. Patent Publication No. 2008/0187954, U.S. Pat. Nos. 6,413,746 and 6,660,501, and International Patent Publication No. WO 2004/009823.

Various mammalian or insect cell culture systems are used to express recombinant polypeptides. Expression of recombinant proteins in mammalian cells can be preferred because such proteins are generally correctly folded, appropriately modified, and biologically functional. Examples of suitable mammalian host cell lines include COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Expression of recombinant proteins in baculovirus also offers a robust method for producing correctly folded and biologically functional proteins. Baculovirus systems for production of heterologous proteins in insect cells are well-known to those of skill in the art (see, e.g., Luckow and Summers, 1988, Bio/Technology, 6:47).

Thus, the present invention provides cells comprising the FZD-binding agents or the Wnt-binding agents described herein. In some embodiments, the cells produce the binding agents (e.g., antibodies or soluble receptors) described herein. In certain embodiments, the cells produce an antibody. In certain embodiments, the cells produce antibody 18R5. In some embodiments, the cells produce a soluble receptor. In some embodiments, the cells produce a FZD-Fc soluble receptor. In some embodiments, the cells produce a FZD8-Fc soluble receptor. In some embodiments, the cells produce a FZD8-Fc soluble receptor 54F28.

The proteins produced by a transformed host can be purified according to any suitable method. Standard methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence, and glutathione-S-transferase can be attached to the protein to allow easy purification by passage over an appropriate affinity column. Isolated proteins can also be physically characterized using such techniques as proteolysis, mass spectrometry (MS), nuclear magnetic resonance (NMR), high performance liquid chromatography (HPLC), and x-ray crystallography.

In some embodiments, supernatants from expression systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. In some embodiments, an anion exchange resin can be employed, for example, a matrix or substrate having pendant diethylaminoethyl (DEAE) groups. The matrices can be acrylamide, agarose, dextran, cellulose, or other types commonly employed in protein purification. In some embodiments, a cation exchange step can be employed. Suitable cation exchangers include various insoluble matrices comprising sulfopropyl or carboxymethyl groups. In some embodiments, a hydroxyapatite media can be employed, including but not limited to, ceramic hydroxyapatite (CHT). In certain embodiments, one or more reverse-phase HPLC steps employing hydrophobic RP-HPLC media, e.g., silica gel having pendant methyl or other aliphatic groups, can be employed to further purify a binding agent. Some or all of the foregoing purification steps, in various combinations, can also be employed to provide a homogeneous recombinant protein.

In some embodiments, recombinant protein produced in bacterial culture can be isolated, for example, by initial extraction from cell pellets, followed by one or more concentration, salting-out, aqueous ion exchange, or size exclusion chromatography steps. HPLC can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying antibodies and other proteins also include, for example, those described in U.S. Patent Publication Nos. 2008/0312425, 2008/0177048, and 2009/0187005.

In certain embodiments, the binding agent is a polypeptide that is not an antibody. A variety of methods for identifying and producing non-antibody polypeptides that bind with high affinity to a protein target are known in the art. See, e.g., Skerra, 2007, Curr. Opin. Biotechnol., 18:295-304; Hosse et al., 2006, Protein Science, 15:14-27; Gill et al., 2006, Curr. Opin. Biotechnol., 17:653-658; Nygren, 2008, FEBS J., 275:2668-76; and Skerra, 2008, FEBS J., 275:2677-83. In certain embodiments, phage display technology may be used to produce and/or identify a FZD-binding or Wnt-binding polypeptide. In certain embodiments, the polypeptide comprises a protein scaffold of a type selected from the group consisting of protein A, protein G, a lipocalin, a fibronectin domain, an ankyrin consensus repeat domain, and thioredoxin.

In certain embodiments, the binding agents can be used in any one of a number of conjugated (i.e. an immunoconjugate or radioconjugate) or non-conjugated forms. In certain embodiments, antibodies can be used in a non-conjugated form to harness the subject's natural defense mechanisms including complement-dependent cytotoxicity and antibody dependent cellular toxicity to eliminate the malignant or cancer cells.

In some embodiments, the binding agent is conjugated to a cytotoxic agent. In some embodiments, the cytotoxic agent is a chemotherapeutic agent including, but not limited to, methotrexate, adriamicin, doxorubicin, melphalan, mitomycin C, chlorambucil, daunorubicin or other intercalating agents. In some embodiments, the cytotoxic agent is an enzymatically active toxin of bacterial, fungal, plant, or animal origin, or fragments thereof, including, but not limited to, diphtheria A chain, nonbinding active fragments of diphtheria toxin, exotoxin A chain, ricin A chain, abrin A chain, modeccin A chain, alpha-sarcin. Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated antibody. A variety of radionuclides are available for the production of radioconjugated antibodies including, but not limited to, ⁹⁰Y, ¹²⁵I, ¹³¹I, ¹²³I, ¹¹¹In, ¹³¹In, ¹⁰⁵Rh, ¹⁵³Sm, ⁶⁷Cu, ⁶⁷Ga, ¹⁶⁶Ho, ¹⁷⁷Lu, ¹⁸⁶Re, ¹⁸⁸Re and ²¹²Bi. In some embodiments, conjugates of an antibody and one or more small molecule toxins, such as a calicheamicin, maytansinoids, a trichothene, and CC1065, and the derivatives of these toxins that have toxin activity, can be produced. In certain embodiments, conjugates of an antibody and a cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In certain embodiments, the Wnt pathway inhibitor (e.g., antibody or soluble receptor) is an antagonist of at least one Wnt protein (i.e., 1, 2, 3, 4, 5, 6, 7, 8, 9, or 10 Wnt proteins). In certain embodiments, the Wnt pathway inhibitor inhibits activity of the Wnt protein(s) to which it binds. In certain embodiments, the Wnt pathway inhibitor inhibits at least about 10%, at least about 20%, at least about 30%, at least about 50%, at least about 75%, at least about 90%, or about 100% of the activity of the human Wnt protein(s) to which it binds.

In certain embodiments, the Wnt pathway inhibitor (e.g., antibody or soluble receptor) inhibits binding of at least one human Wnt to an appropriate receptor. In certain embodiments, the Wnt pathway inhibitor inhibits binding of at least one human Wnt protein to one or more human FZD proteins. In some embodiments, the at least one Wnt protein is selected from the group consisting of: Wnt1, Wnt2, Wnt2b/13, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and Wnt16. In some embodiments, the one or more human FZD proteins are selected from the group consisting of: FZD1, FZD2, FZD3, FZD4, FZD5, FZD6, FZD7, FZD8, FZD9, and FZD10. In certain embodiments, the Wnt pathway inhibitor inhibits binding of one or more Wnt proteins to FZD1, FZD2, FZD4, FZD5, FZD7, and/or FZD8. In certain embodiments, the Wnt pathway inhibitor inhibits binding of one or more Wnt proteins to FZD8. In certain embodiments, the inhibition of binding of a particular Wnt to a FZD protein by a Wnt pathway inhibitor is at least about 10%, at least about 25%, at least about 50%, at least about 75%, at least about 90%, or at least about 95%. In certain embodiments, an agent that inhibits binding of a Wnt to a FZD protein, also inhibits Wnt pathway signaling. In certain embodiments, a Wnt pathway inhibitor that inhibits human Wnt pathway signaling is an antibody. In certain embodiments, a Wnt pathway inhibitor that inhibits human Wnt pathway signaling is a FZD-Fc soluble receptor. In certain embodiments, a Wnt pathway inhibitor that inhibits human Wnt pathway signaling is a FZD8-Fc soluble receptor. In certain embodiments, a Wnt pathway inhibitor that inhibits human Wnt pathway signaling is soluble receptor 54F28.

In certain embodiments, the Wnt pathway inhibitors (e.g., antibody or soluble receptor) described herein are antagonists of at least one human Wnt protein and inhibit Wnt activity. In certain embodiments, the Wnt pathway inhibitor inhibits Wnt activity by at least about 10%, at least about 20%, at least about 30%, at least about 50%, at least about 75%, at least about 90%, or about 100%. In some embodiments, the Wnt pathway inhibitor inhibits activity of one, two, three, four, five or more Wnt proteins. In some embodiments, the Wnt pathway inhibitor inhibits activity of at least one human Wnt protein selected from the group consisting of: Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and Wnt16. In some embodiments, the Wnt-binding agent binds at least one Wnt protein selected from the group consisting of Wnt1, Wnt2, Wnt2b, Wnt3, Wnt3a, Wnt7a, Wnt7b, Wnt8a, Wnt8b, Wnt10a, and Wnt10b. In certain embodiments, the at least one Wnt protein is selected from the group consisting of Wnt1, Wnt2, Wnt2b, Wnt3, Wnt3a, Wnt8a, Wnt8b, Wnt10a, and Wnt10b. In certain embodiments, a Wnt pathway inhibitor that inhibits human Wnt activity is an antibody. In certain embodiments, a Wnt pathway inhibitor that inhibits human Wnt activity is a FZD-Fc soluble receptor. In certain embodiments, a Wnt pathway inhibitor that inhibits human Wnt activity is a FZD8-Fc soluble receptor. In certain embodiments, a Wnt pathway inhibitor that inhibits human Wnt activity is soluble receptor 54F28.

In certain embodiments, the Wnt pathway inhibitor described herein is an antagonist of at least one human FZD protein and inhibits FZD activity. In certain embodiments, the Wnt pathway inhibitor inhibits FZD activity by at least about 10%, at least about 20%, at least about 30%, at least about 50%, at least about 75%, at least about 90%, or about 100%. In some embodiments, the Wnt pathway inhibitor inhibits activity of one, two, three, four, five or more FZD proteins. In some embodiments, the Wnt pathway inhibitor inhibits activity of at least one human FZD protein selected from the group consisting of: FZD1, FZD2, FZD3, FZD4, FZD5, FZD6, FZD7, FZD8, FZD9, and FZD10. In certain embodiments, the Wnt pathway inhibitor inhibits activity of FZD1, FZD2, FZD4, FZD5, FZD7, and/or FZD8. In certain embodiments, the Wnt pathway inhibitor inhibits activity of FZD8. In some embodiments, the Wnt pathway inhibitor is an anti-FZD antibody. In certain embodiments, the Wnt pathway inhibitor is anti-FZD antibody 18R5.

In certain embodiments, the Wnt pathway inhibitor described herein is an antagonist of at least one human Wnt protein and inhibits Wnt signaling. In certain embodiments, the Wnt pathway inhibitor inhibits Wnt signaling by at least about 10%, at least about 20%, at least about 30%, at least about 50%, at least about 75%, at least about 90%, or about 100%. In some embodiments, the Wnt pathway inhibitor inhibits signaling by one, two, three, four, five or more Wnt proteins. In some embodiments, the Wnt pathway inhibitor inhibits signaling of at least one Wnt protein selected from the group consisting of Wnt1, Wnt2, Wnt2b, Wnt3, Wnt3a, Wnt7a, Wnt7b, Wnt8a, Wnt8b, Wnt10a, and Wnt10b. In certain embodiments, a Wnt pathway inhibitor that inhibits Wnt signaling is an antibody. In certain embodiments, a Wnt pathway inhibitor that inhibits Wnt signaling is a soluble receptor. In certain embodiments, a Wnt pathway inhibitor that inhibits Wnt signaling is a FZD-Fc soluble receptor. In certain embodiments, a Wnt pathway inhibitor that inhibits Wnt signaling is a FZD8-Fc soluble receptor. In certain embodiments, a Wnt pathway inhibitor that inhibits Wnt signaling is soluble receptor 54F28.

In certain embodiments, a Wnt pathway inhibitor described herein is an antagonist of β-catenin signaling. In certain embodiments, the Wnt pathway inhibitor inhibits β-catenin signaling by at least about 10%, at least about 20%, at least about 30%, at least about 50%, at least about 75%, at least about 90%, or about 100%. In certain embodiments, a Wnt pathway inhibitor that inhibits β-catenin signaling is an antibody. In certain embodiments, a Wnt pathway inhibitor that inhibits β-catenin signaling is an anti-FZD antibody. In certain embodiments, a Wnt pathway inhibitor that inhibits β-catenin signaling is antibody 18R5. In certain embodiments, a Wnt pathway inhibitor that inhibits β-catenin signaling is a soluble receptor. In certain embodiments, a Wnt pathway inhibitor that inhibits β-catenin signaling is a FZD-Fc soluble receptor. In certain embodiments, a Wnt pathway inhibitor that inhibits β-catenin signaling is a FZD8-Fc soluble receptor.

In certain embodiments, the Wnt pathway inhibitor described herein inhibits binding of at least one Wnt protein to a receptor. In certain embodiments, the Wnt pathway inhibitor inhibits binding of at least one human Wnt protein to one or more of its receptors. In some embodiments, the Wnt pathway inhibitor inhibits binding of at least one Wnt protein to at least one FZD protein. In some embodiments, the Wnt-binding agent inhibits binding of at least one Wnt protein to FZD1, FZD2, FZD3, FZD4, FDZ5, FDZ6, FDZ7, FDZ8, FDZ9, and/or FZD10. In certain embodiments, the inhibition of binding of at least one Wnt to at least one FZD protein is at least about 10%, at least about 25%, at least about 50%, at least about 75%, at least about 90%, or at least about 95%. In certain embodiments, a Wnt pathway inhibitor that inhibits binding of at least one Wnt to at least one FZD protein further inhibits Wnt pathway signaling and/or β-catenin signaling. In certain embodiments, a Wnt pathway inhibitor that inhibits binding of at least one human Wnt to at least one FZD protein is an antibody. In certain embodiments, a Wnt pathway inhibitor that inhibits binding of at least one human Wnt to at least one FZD protein is an anti-FZD antibody. In certain embodiments, a Wnt pathway inhibitor that inhibits binding of at least one human Wnt to at least one FZD protein is antibody 18R5. In certain embodiments, a Wnt pathway inhibitor that inhibits binding of at least one human Wnt to at least one FZD protein is a soluble receptor. In certain embodiments, a Wnt pathway inhibitor that inhibits binding of at least one human Wnt to at least one FZD protein is a FZD-Fc soluble receptor. In certain embodiments, a Wnt pathway inhibitor that inhibits binding of at least one human Wnt to at least one FZD protein is a FZD8-Fc soluble receptor. In certain embodiments, a Wnt pathway inhibitor that inhibits binding of at least one human Wnt to at least one FZD protein is FZD8-Fc soluble receptor 54F28.

In certain embodiments, the Wnt pathway inhibitor described herein blocks binding of at least one Wnt to a receptor. In certain embodiments, the Wnt pathway inhibitor blocks binding of at least one human Wnt protein to one or more of its receptors. In some embodiments, the Wnt pathway inhibitor blocks binding of at least one Wnt to at least one FZD protein. In some embodiments, the Wnt pathway inhibitor blocks binding of at least one Wnt protein to FZD1, FZD2, FZD3, FZD4, FDZ5, FDZ6, FDZ7, FDZ8, FDZ9, and/or FZD10. In certain embodiments, the blocking of binding of at least one Wnt to at least one FZD protein is at least about 10%, at least about 25%, at least about 50%, at least about 75%, at least about 90%, or at least about 95%. In certain embodiments, a Wnt pathway inhibitor that blocks binding of at least one Wnt protein to at least one FZD protein further inhibits Wnt pathway signaling and/or β-catenin signaling. In certain embodiments, a Wnt pathway inhibitor that blocks binding of at least one human Wnt to at least one FZD protein is an antibody. In certain embodiments, a Wnt pathway inhibitor that blocks binding of at least one human Wnt to at least one FZD protein is an anti-FZD antibody. In certain embodiments, a Wnt pathway inhibitor that blocks binding of at least one human Wnt to at least one FZD protein is antibody 18R5. In certain embodiments, a Wnt pathway inhibitor that blocks binding of at least one human Wnt to at least one FZD protein is a soluble receptor. In certain embodiments, a Wnt pathway inhibitor that blocks binding of at least one human Wnt to at least one FZD protein is a FZD-Fc soluble receptor. In certain embodiments, a Wnt pathway inhibitor that blocks binding of at least one human Wnt to at least one FZD protein is a FZD8-Fc soluble receptor. In certain embodiments, a Wnt pathway inhibitor that blocks binding of at least one human Wnt to at least one FZD protein is soluble receptor 54F28.

In certain embodiments, the Wnt pathway inhibitor described herein inhibits Wnt pathway signaling. It is understood that a Wnt pathway inhibitor that inhibits Wnt pathway signaling may, in certain embodiments, inhibit signaling by one or more receptors in the Wnt signaling pathway but not necessarily inhibit signaling by all receptors. In certain alternative embodiments, Wnt pathway signaling by all human receptors may be inhibited. In certain embodiments, Wnt pathway signaling by one or more receptors selected from the group consisting of FZD1, FZD2, FZD3, FZD4, FDZ5, FDZ6, FDZ7, FDZ8, FDZ9, and FZD10 is inhibited. In certain embodiments, the inhibition of Wnt pathway signaling by a Wnt pathway inhibitor is a reduction in the level of Wnt pathway signaling of at least about 10%, at least about 25%, at least about 50%, at least about 75%, at least about 90%, or at least about 95%. In some embodiments, a Wnt pathway inhibitor that inhibits Wnt pathway signaling is an antibody. In some embodiments, a Wnt pathway inhibitor that inhibits Wnt pathway signaling is an anti-FZD antibody. In some embodiments, a Wnt pathway inhibitor that inhibits Wnt pathway signaling is antibody 18R5. In some embodiments, a Wnt pathway inhibitor that inhibits Wnt pathway signaling is a soluble receptor. In some embodiments, a Wnt pathway inhibitor that inhibits Wnt pathway signaling is a FZD-Fc soluble receptor. In some embodiments, a Wnt pathway inhibitor that inhibits Wnt pathway signaling is a FZD8-Fc soluble receptor. In some embodiments, a Wnt pathway inhibitor that inhibits Wnt pathway signaling is soluble receptor 54F28.

In certain embodiments, the Wnt pathway inhibitor described herein inhibits activation of β-catenin. It is understood that a Wnt pathway inhibitor that inhibits activation of β-catenin may, in certain embodiments, inhibit activation of β-catenin by one or more receptors, but not necessarily inhibit activation of β-catenin by all receptors. In certain alternative embodiments, activation of β-catenin by all human receptors may be inhibited. In certain embodiments, activation of β-catenin by one or more receptors selected from the group consisting of FZD1, FZD2, FZD3, FZD4, FDZ5, FDZ6, FDZ7, FDZ8, FDZ9, and FZD10 is inhibited. In certain embodiments, the inhibition of activation of β-catenin by a Wnt-binding agent is a reduction in the level of activation of β-catenin of at least about 10%, at least about 25%, at least about 50%, at least about 75%, at least about 90%, or at least about 95%. In some embodiments, a Wnt pathway inhibitor that inhibits activation of β-catenin is an antibody. In some embodiments, a Wnt pathway inhibitor that inhibits activation of β-catenin is an anti-FZD antibody. In some embodiments, a Wnt pathway inhibitor that inhibits activation of β-catenin is antibody 18R5. In some embodiments, a Wnt pathway inhibitor that inhibits activation of β-catenin is a soluble receptor. In some embodiments, a Wnt pathway inhibitor that inhibits activation of β-catenin is a FZD-Fc soluble receptor. In some embodiments, a Wnt pathway inhibitor that inhibits activation of β-catenin is a FZD8-Fc soluble receptor. In some embodiments, a Wnt pathway inhibitor that inhibits activation of β-catenin is soluble receptor 54F28.

In vivo and in vitro assays for determining whether a Wnt pathway inhibitor inhibits β-catenin signaling are known in the art. For example, cell-based, luciferase reporter assays utilizing a TCF/Luc reporter vector containing multiple copies of the TCF-binding domain upstream of a firefly luciferase reporter gene may be used to measure β-catenin signaling levels in vitro (Gazit et al., 1999, Oncogene, 18; 5959-66; TOPflash, Millipore, Billerica Mass.). The level of β-catenin signaling in the presence of one or more Wnt proteins (e.g., Wnt(s) expressed by transfected cells or provided by Wnt-conditioned media) in the presence of a binding agent is compared to the level of signaling without the binding agent present. In addition to the TCF/Luc reporter assay, the effect of a binding agent (or candidate agent) on β-catenin signaling may be measured in vitro or in vivo by measuring the effect of the agent on the level of expression of β-catenin-regulated genes, such as c-myc (He et al., 1998, Science, 281:1509-12), cyclin D1 (Tetsu et al., 1999, Nature, 398:422-6), and/or fibronectin (Gradl et al. 1999, Mol. Cell Biol., 19:5576-87). In certain embodiments, the effect of a binding agent on β-catenin signaling may also be assessed by measuring the effect of the agent on the phosphorylation state of Dishevelled-1, Dishevelled-2, Dishevelled-3, LRP5, LRP6, and/or β-catenin.

In certain embodiments, a Wnt pathway inhibitor has one or more of the following effects: inhibit proliferation of tumor cells, inhibit tumor growth, reduce the frequency of cancer stem cells in a tumor, reduce the tumorigenicity of a tumor, reduce the tumorigenicity of a tumor by reducing the frequency of cancer stem cells in the tumor, trigger cell death of tumor cells, induce cells in a tumor to differentiate, differentiate tumorigenic cells to a non-tumorigenic state, induce expression of differentiation markers in the tumor cells, prevent metastasis of tumor cells, or decrease survival of tumor cells.

In certain embodiments, a Wnt pathway inhibitor is capable of inhibiting tumor growth. In certain embodiments, a Wnt pathway inhibitor is capable of inhibiting tumor growth in vivo (e.g., in a xenograft mouse model, and/or in a human having cancer). In some embodiments, the tumor is a tumor selected from the group consisting of colorectal tumor, colon tumor, pancreatic tumor, lung tumor, ovarian tumor, liver tumor, breast tumor, kidney tumor, prostate tumor, gastrointestinal tumor, melanoma, cervical tumor, bladder tumor, glioblastoma, and head and neck tumor. In certain embodiments, the tumor is melanoma. In certain embodiments, the tumor is a colorectal tumor. In certain embodiments, the tumor is a pancreatic tumor. In certain embodiments, the tumor is a breast tumor. In certain embodiments, the tumor is a Wnt-dependent tumor. In some embodiments, the tumor has a mutation in a component of the MAPK pathway. In some embodiments, the tumor has a mutated Ras gene and/or protein. In some embodiments, the tumor has a mutated K-Ras or N-Ras gene and/or protein. In some embodiments, the tumor has a mutated B-Raf gene and/or protein.

In certain embodiments, a Wnt pathway inhibitor is capable of reducing the tumorigenicity of a tumor. In certain embodiments, a Wnt pathway inhibitor is capable of reducing the tumorigenicity of a tumor comprising cancer stem cells in an animal model, such as a mouse xenograft model. In certain embodiments, the number or frequency of cancer stem cells in a tumor is reduced by at least about two-fold, about three-fold, about five-fold, about ten-fold, about 50-fold, about 100-fold, or about 1000-fold. In certain embodiments, the reduction in the number or frequency of cancer stem cells is determined by limiting dilution assay using an animal model. Additional examples and guidance regarding the use of limiting dilution assays to determine a reduction in the number or frequency of cancer stem cells in a tumor can be found, e.g., in International Publication Number WO 2008/042236, and U.S. Patent Publication Nos. 2008/0064049, and 2008/0178305.

In certain embodiments, the Wnt pathway inhibitors described herein are active in vivo for at least 1 hour, at least about 2 hours, at least about 5 hours, at least about 10 hours, at least about 24 hours, at least about 2 days, at least about 3 days, at least about 1 week, or at least about 2 weeks. In certain embodiments, the Wnt pathway inhibitor is an IgG (e.g., IgG1 or IgG2) antibody that is active in vivo for at least 1 hour, at least about 2 hours, at least about 5 hours, at least about 10 hours, at least about 24 hours, at least about 2 days, at least about 3 days, at least about 1 week, or at least about 2 weeks. In certain embodiments, the Wnt pathway inhibitor is a fusion protein that is active in vivo for at least 1 hour, at least about 2 hours, at least about 5 hours, at least about 10 hours, at least about 24 hours, at least about 2 days, at least about 3 days, at least about 1 week, or at least about 2 weeks.

In certain embodiments, the Wnt pathway inhibitors described herein have a circulating half-life in mice, cynomolgus monkeys, or humans of at least about 5 hours, at least about 10 hours, at least about 24 hours, at least about 2 days, at least about 3 days, at least about 1 week, or at least about 2 weeks. In certain embodiments, the Wnt pathway inhibitor is an IgG (e.g., IgG1 or IgG2) antibody that has a circulating half-life in mice, cynomolgus monkeys, or humans of at least about 5 hours, at least about 10 hours, at least about 24 hours, at least about 2 days, at least about 3 days, at least about 1 week, or at least about 2 weeks. In certain embodiments, the Wnt pathway inhibitor is a fusion protein that has a circulating half-life in mice, cynomolgus monkeys, or humans of at least about 5 hours, at least about 10 hours, at least about 24 hours, at least about 2 days, at least about 3 days, at least about 1 week, or at least about 2 weeks. Methods of increasing (or decreasing) the half-life of agents such as polypeptides and antibodies are known in the art. For example, known methods of increasing the circulating half-life of IgG antibodies include the introduction of mutations in the Fc region which increase the pH-dependent binding of the antibody to the neonatal Fc receptor (FcRn) at pH 6.0 (see, e.g., U.S. Patent Publication Nos. 2005/0276799, 2007/0148164, and 2007/0122403). Known methods of increasing the circulating half-life of antibody fragments lacking the Fc region include such techniques as PEGylation.

### III. MAPK PATHWAY INHIBITORS

The present invention provides Wnt pathway inhibitors for use in combination therapy with MAPK pathway inhibitors for inhibiting tumor growth and/or for the treatment of cancer. In some embodiments, a MAPK pathway inhibitor is a small molecule. In some embodiments, a MAPK pathway inhibitor is selected from the group consisting of a MEK inhibitor, a Ras inhibitor, a Raf inhibitor, and a ERK inhibitor. In some embodiments, a MAPK pathway inhibitor is a MEK inhibitor. In some embodiments, a MEK inhibitor is selected from the group consisting of: BAY 86-9766 (RDEA119), PD0325901, CI-1040 (PD184352), PD98059, PD318088, GSK1120212 (JTP-74057), AZD8330 (ARRY-424704), AZD6244 (ARRY-142886), ARRY-162, ARRY-300, AS703026, U0126, GDC-0973, GDC-0623, CH4987655, and TAK-733. In some embodiments, a MEK inhibitor is BAY 86-9766. In some embodiments, a MAPK pathway inhibitor is a Raf inhibitor. In some embodiments, a Raf inhibitor is selected from the group consisting of: GDC-0879, PLX-4720, PLX-4032 (vemurafenib), RAF265, BAY 73-4506, BAY 43-9006 (sorafenib), SB590885, XL281 (BMS-908662), and GSK 2118436436. In some embodiments, a Raf inhibitor is BAY 43-9006. In some embodiments, a Raf inhibitor is PLX-4032. In some embodiments, a Raf inhibitor is GDC-0879. In some embodiments, a MAPK pathway inhibitor is a Ras inhibitor. In some embodiments, the Ras inhibitor is farnesylthiosalicylic acid (FTS). In some embodiments, a MAPK pathway inhibitor is an ERK inhibitor.

### IV. METHODS OF USE AND PHARMACEUTICAL COMPOSITIONS

The Wnt pathway inhibitors (e.g., Wnt-binding agents and FDZ-binding agents) of the invention in combination with MAPK pathway inhibitors are useful in a variety of applications including, but not limited to, therapeutic treatment methods, such as the treatment of cancer. In certain embodiments, the combination of a Wnt pathway inhibitor and a MAPK pathway inhibitor is useful in methods of inhibiting Wnt signaling (e.g., canonical Wnt signaling), inhibiting MAPK signaling, inhibiting tumor growth, inducing differentiation, reducing tumor volume, reducing cancer stem cell frequency, and/or reducing the tumorigenicity of a tumor. The methods of use may be in vitro, ex vivo, or in vivo methods.

In some embodiments, a Wnt pathway inhibitor (e.g., Wnt-binding agents or FDZ-binding agents) in combination with a MAPK pathway inhibitor is used in a method of treating a disease associated with Wnt pathway activation. In some embodiments, the disease is a disease dependent upon Wnt signaling. In particular embodiments, the Wnt signaling is canonical Wnt signaling.

In some embodiments, a Wnt pathway inhibitor (e.g., Wnt-binding agents or FDZ-binding agents) in combination with a MAPK pathway inhibitor is used in a method of treating a disease associated MAPK pathway activation. In some embodiments, a Wnt pathway inhibitor in combination with a MAPK pathway inhibitor is used in a method of treating a disease associated with activation of a component of the MAPK pathway. In some embodiments, the component of the MAPK pathway is a Ras protein, a Raf protein, a MEK protein, or a ERK protein.

In some embodiments, the disease treated with a combination of a Wnt pathway inhibitor (e.g., Wnt-binding agents or FDZ-binding agents) and a MAPK pathway inhibitor is cancer. In certain embodiments, the cancer is characterized by Wnt-dependent tumors. In certain embodiments, the cancer is characterized by tumors expressing or over-expressing one or more Wnt proteins. In certain embodiments, the cancer is characterized by tumors expressing or over-expressing one or more FZD proteins.

The present invention provides a method of treating cancer in a subject comprising administering to the subject a therapeutically effective amount of a Wnt pathway inhibitor in combination with a therapeutically effective amount of a MAPK pathway inhibitor. In some embodiments, the methods comprise using the Wnt-binding agents, FZD-binding agents, and/or MAPK pathway inhibitors described herein. In certain embodiments, the cancer is a cancer selected from the group consisting of colorectal cancer, pancreatic cancer, lung cancer, ovarian cancer, liver cancer, breast cancer, kidney cancer, prostate cancer, gastrointestinal cancer, melanoma, cervical cancer, bladder cancer, glioblastoma, and head and neck cancer. In certain embodiments, the cancer is melanoma. In certain embodiments, the cancer is lung cancer. In certain embodiments, the cancer is pancreatic cancer. In certain embodiments, the cancer is colorectal cancer. In certain embodiments, the cancer is breast cancer. In some embodiments, the cancer has a mutation in a component of the MAPK pathway. In some embodiments, the cancer has a mutated Ras gene and/or protein. In some embodiments, the cancer has a mutated K-Ras or N-Ras gene and/or protein. In some embodiments, the cancer has a mutated B-Raf gene and/or protein. In certain embodiments, the subject is a human.

The present invention further provides a method for inhibiting tumor growth comprising contacting tumor cells with an effective amount of a Wnt pathway inhibitor in combination with an effective amount of a MAPK pathway inhibitor. In some embodiments, the methods comprise using the Wnt-binding agents, FZD-binding agents, and/or MAPK pathway inhibitors described herein. In certain embodiments, the method of inhibiting tumor growth comprises contacting the tumor or tumor cell with a combination of a Wnt pathway inhibitor and a MAPK pathway inhibitor in vitro. For example, in some embodiments, an immortalized cell line or a cancer cell line that expresses the targeted Wnt or FZD protein(s) is cultured in medium to which is added the Wnt/FZD binding agent in combination with a MAPK pathway inhibitor to inhibit tumor cell growth. In some embodiments, tumor cells are isolated from a patient sample such as, for example, a tissue biopsy, pleural effusion, or blood sample and cultured in medium to which is added the Wnt/FZD binding agent in combination with a MAPK pathway inhibitor to inhibit tumor cell growth.

In some embodiments, the method of inhibiting tumor growth comprises contacting the tumor or tumor cells with a combination of a Wnt pathway inhibitor and a MAPK pathway inhibitor in vivo. In certain embodiments, contacting a tumor or tumor cell with a combination of a Wnt pathway inhibitor and a MAPK pathway inhibitor is undertaken in an animal model. For example, a combination of a Wnt pathway inhibitor and a MAPK pathway inhibitor may be administered to immunocompromised mice (e.g., NOD/SCID mice) which bear xenograft tumors to inhibit growth of the tumors. In certain embodiments, cancer stem cells are isolated from a patient sample such as, for example, a tissue biopsy, pleural effusion, or blood sample and injected into immunocompromised mice that are then administered a combination of a Wnt pathway inhibitor and a MAPK pathway inhibitor to inhibit tumor cell growth. In some embodiments, the combination of a Wnt pathway inhibitor and a MAPK pathway inhibitor is administered at the same time or shortly after introduction of cells into the animal to prevent tumor growth (preventative model). In some embodiments, the combination of a Wnt pathway inhibitor and a MAPK pathway inhibitor is administered after the cells have grown to a tumor of a specific size to inhibit and/or reduce tumor growth (therapeutic model).

The invention also provides a method of inhibiting tumor growth in a subject comprising administering to the subject a therapeutically effective amount of a Wnt pathway inhibitor in combination with a therapeutically effective amount of a MAPK pathway inhibitor. In some embodiments, the methods comprise using the Wnt-binding agents, FZD-binding agents, and/or MAPK pathway inhibitors described herein. In certain embodiments, the subject is a human. In certain embodiments, the subject has a tumor or has had a tumor removed. In some embodiments, the subject has a tumor that has metastasized. In some embodiments, the subject has had prior therapeutic treatment. In some embodiments, the subject has been treated with a MAPK pathway inhibitor. In some embodiments, the subject has been treated with a B-Raf inhibitor.

The invention also provides a method of inhibiting invasiveness of a tumor in a subject comprising administering to the subject a therapeutically effective amount of a Wnt pathway inhibitor in combination with a therapeutically effective amount of a MAPK pathway inhibitor. In some embodiments, the methods comprise using the Wnt-binding agents, FZD-binding agents, and/or MAPK pathway inhibitors described herein. In some embodiments, the inhibition of invasiveness comprises increasing E-cadherin expression. In certain embodiments, the subject is a human. In certain embodiments, the subject has a tumor or has had a tumor removed.

The invention also provides a method of reducing or preventing metastasis in a subject comprising administering to the subject a therapeutically effective amount of a Wnt pathway inhibitor in combination with a therapeutically effective amount of a MAPK pathway inhibitor. In some embodiments, the methods comprise using the Wnt-binding agents, FZD-binding agents, and/or MAPK pathway inhibitors described herein. In some embodiments, the reduction or prevention of metastasis comprises inhibiting invasiveness of a tumor. In some embodiments, the reduction or prevention of metastasis comprises inhibiting invasiveness of a tumor by increasing E-cadherin expression. In certain embodiments, the subject is a human. In certain embodiments, the subject has a tumor or has had a tumor removed.

In certain embodiments, the tumor is a tumor in which Wnt signaling is active. In certain embodiments, the Wnt signaling that is active is canonical Wnt signaling. In certain embodiments, the tumor is a Wnt-dependent tumor.

In certain embodiments, the tumor expresses one or more human Wnt proteins to which a Wnt-binding agent binds. In certain embodiments, the tumor over-expresses one or more human Wnt protein(s). In certain embodiments, the tumor over-expresses one or more human Wnt protein(s) as compared to the Wnt protein expression in normal tissue of the same tissue type. In certain embodiments, the tumor over-expresses one or more human Wnt protein(s) as compared to the Wnt protein expression in at least one other tumor. In some embodiments, the tumor over-expresses Wnt3 or Wnt3a. In certain embodiments, the tumor expresses one or more human FZD proteins to which a FZD-binding agent binds. In certain embodiments, the tumor over-expresses one or more human FZD proteins.

In certain embodiments, the tumor is a tumor in which MAPK pathway signaling is active. In some embodiments, the MAPK pathway signaling is active due to mutation of a MAPK pathway component. In some embodiments, the MAPK pathway component is B-Raf. In some embodiments, the MAPK pathway component is K-Ras. In some embodiments, the MAPK pathway component is N-Ras.

In certain embodiments, the tumor is a tumor selected from the group consisting of colorectal tumor, pancreatic tumor, lung tumor, ovarian tumor, liver tumor, breast tumor, kidney tumor, prostate tumor, gastrointestinal tumor, melanoma, cervical tumor, bladder tumor, glioblastoma, and head and neck tumor. In certain embodiments, the tumor is a melanoma. In certain embodiments, the tumor is a lung tumor. In certain embodiments, the tumor is a colorectal tumor. In certain embodiments, the tumor is a pancreatic tumor. In certain embodiments, the tumor is a breast tumor. In some embodiments, the tumor has a mutation in a component of the MAPK pathway. In some embodiments, the tumor has a mutated Ras gene and/or protein. In some embodiments, the tumor has a mutated K-Ras or N-Ras gene and/or protein. In some embodiments, the tumor has a mutated B-Raf gene and/or protein.

The invention also provides a method of inhibiting Wnt signaling in a cell comprising contacting the cell with an effective amount of a Wnt pathway inhibitor. In some embodiments, the method further inhibits MAPK pathway signaling in the cells comprising contacting the cell with a MAPK pathway inhibitor. In certain embodiments, the cell is a tumor cell. In certain embodiments, the method is an in vivo method wherein the step of contacting the cell with the inhibitor(s) comprises administering a therapeutically effective amount of the inhibitor(s) to the subject. In some alternative embodiments, the method is an in vitro or ex vivo method. In certain embodiments, the Wnt signaling that is inhibited is canonical Wnt signaling. In certain embodiments, the Wnt signaling is signaling by Wnt1, Wnt2, Wnt3, Wnt3a, Wnt7a, Wnt7b, and/or Wnt10b. In certain embodiments, the Wnt signaling is signaling by Wnt1, Wnt3a, Wnt7b, and/or Wnt10b.

In addition, the invention provides a method of reducing the tumorigenicity of a tumor in a subject, comprising administering to a subject a therapeutically effective amount of a Wnt pathway inhibitor in combination with a therapeutically effective amount of a MAPK pathway inhibitor. In certain embodiments, the tumor comprises cancer stem cells. In some embodiments, the tumorigenicity of a tumor is reduced by reducing the frequency of cancer stem cells in the tumor. In some embodiments, the methods comprise using the Wnt-binding agents, FZD-binding agents, and/or MAPK pathway inhibitors described herein. In certain embodiments, the frequency of cancer stem cells in the tumor is reduced by administration of the Wnt pathway inhibitor. In some embodiments, the tumorigenicity of the tumor is reduced by inducing differentiation of the tumor cells.

The invention also provides a method of reducing cancer stem cell frequency in a tumor comprising cancer stem cells, the method comprising administering to a subject a therapeutically effective amount of a Wnt pathway inhibitor in combination with a therapeutically effective amount of a MAPK pathway inhibitor. In some embodiments, the methods comprise using the Wnt-binding agents, FZD-binding agents, and/or MAPK pathway inhibitors described herein. In certain embodiments, the Wnt pathway inhibitor in combination with a MAPK pathway inhibitor is capable of reducing the tumorigenicity of a tumor comprising cancer stem cells in an animal model, such as a mouse xenograft model. In certain embodiments, the number or frequency of cancer stem cells in a treated tumor is reduced by at least about two-fold, about three-fold, about five-fold, about ten-fold, about 50-fold, about 100-fold, or about 1000-fold as compared to the number or frequency of cancer stem cells in an untreated tumor. In certain embodiments, the reduction in the number or frequency of cancer stem cells is determined by limiting dilution assay using an animal model.

The present invention provides methods of treating a human subject, comprising: (a) determining if the subject has a tumor comprising a mutation in the MAPK pathway, and (b) administering to the subject (e.g., a subject in need of treatment) a therapeutically effective amount of a Wnt pathway inhibitor in combination with a therapeutically effective amount of a MAPK pathway inhibitor. In certain embodiments, the subject has a cancerous tumor. In certain embodiments, the subject has had a tumor removed. In some embodiments, the subject has been previously treated with a MAPK pathway inhibitor. In some embodiments, the subject has been previously treated with a B-Raf inhibitor.

The present invention further provides methods of treating a human subject, comprising: (a) selecting a subject for treatment based, at least in part, on the subject having a tumor that comprises a wild-type B-Raf or a B-Raf mutation, and (b) administering to the subject a therapeutically effective amount of a Wnt pathway inhibitor in combination with a therapeutically effective amount of a MAPK pathway inhibitor. In certain embodiments, the subject has a cancerous tumor. In certain embodiments, the subject has had a tumor removed. In some embodiments, the subject has been previously treated with a MAPK pathway inhibitor. In some embodiments, the subject has been previously treated with a B-Raf inhibitor.

The present invention further provides methods of treating a human subject, comprising: (a) selecting a subject for treatment based, at least in part, on the subject having a tumor that comprises a wild-type N-Ras or an N-Ras mutation, and (b) administering to the subject a therapeutically effective amount of a Wnt pathway inhibitor in combination with a therapeutically effective amount of a MAPK pathway inhibitor. In certain embodiments, the subject has a cancerous tumor. In certain embodiments, the subject has had a tumor removed. In some embodiments, the subject has been previously treated with a MAPK pathway inhibitor. In some embodiments, the subject has been previously treated with a B-Raf inhibitor.

The present invention further provides methods of treating a human subject, comprising: (a) selecting a subject for treatment based, at least in part, on the subject having a tumor that comprises a wild-type K-Ras or a K-Ras mutation, and (b) administering to the subject a therapeutically effective amount of a Wnt pathway inhibitor in combination with a therapeutically effective amount of a MAPK pathway inhibitor. In certain embodiments, the subject has a cancerous tumor. In certain embodiments, the subject has had a tumor removed. In some embodiments, the subject has been previously treated with a MAPK pathway inhibitor. In some embodiments, the subject has been previously treated with a B-Raf inhibitor.

The present invention further provides methods of treating a human subject who has a tumor which is substantially non-responsive to at least one B-Raf inhibitor, comprising administering to the subject a therapeutically effective amount of a Wnt pathway inhibitor in combination with a therapeutically effective amount of a MAPK pathway inhibitor. In certain embodiments, the subject has a cancerous tumor. In certain embodiments, the subject has had a tumor removed. In some embodiments, the subject has been previously treated with a MAPK pathway inhibitor. In some embodiments, the subject has been previously treated with a B-Raf inhibitor. In some embodiments, the subject has a wild-type B-Raf.

In some embodiments, the tumor comprising a B-Raf mutation is substantially non-responsive to at least one B-Raf inhibitor. In some embodiments, the tumor is substantially non-responsive to at least one B-Raf inhibitor which is a small molecule compound. In some embodiments, the tumor is substantially non-responsive to at least one B-Raf inhibitor which is selected from the group consisting of: GDC-0879, PLX-4720, PLX-4032 (vemurafenib; ZELBORAF), RAF265, BAY 73-4506, BAY 43-9006 (sorafenib), SB590885, XL281 (BMS-908662), and GSK 2118436436. In some embodiments, the tumor is substantially non-responsive to PLX-4032.

The present invention further provides methods of treating a human subject, comprising: (a) selecting a subject for treatment based, at least in part, on the subject having a tumor that is substantially non-responsive to at least one B-Raf inhibitor; and (b) administering to the subject a therapeutically effective amount of a Wnt pathway inhibitor in combination with a therapeutically effective amount of a MAPK pathway inhibitor. In some embodiments, the tumor comprises at least one B-Raf mutation. In some embodiments, the tumor comprises a wild-type B-Raf. In some embodiments, the subject has been previously treated with a B-Raf inhibitor. In some embodiments, the tumor is substantially non-responsive to at least one B-Raf inhibitor which is a small molecule compound. In some embodiments, the tumor is substantially non-responsive to at least one B-Raf inhibitor which is selected from the group consisting of: GDC-0879, PLX-4720, PLX-4032 (vemurafenib; ZELBORAF), RAF265, BAY 73-4506, BAY 43-9006 (sorafenib), SB590885, XL281 (BMS-908662), and GSK 2118436436. In some embodiments, the tumor is substantially non-responsive to PLX-4032.

The present invention further provides methods of treating a human subject, comprising: (a) identifying a subject that has a tumor that is substantially non-responsive to at least one B-Raf inhibitor, and (b) administering to the subject a therapeutically effective amount of a Wnt pathway inhibitor in combination with a therapeutically effective amount of a MAPK pathway inhibitor. In some embodiments, the tumor comprises at least one B-Raf mutation. In some embodiments, the tumor comprises a wild-type B-Raf. In some embodiments, the subject has been previously treated with a B-Raf inhibitor. In some embodiments, the tumor is substantially non-responsive to at least one B-Raf inhibitor which is a small molecule compound. In some embodiments, the tumor is substantially non-responsive to at least one B-Raf inhibitor which is selected from the group consisting of: GDC-0879, PLX-4720, PLX-4032 (vemurafenib; ZELBORAF), RAF265, BAY 73-4506, BAY 43-9006 (sorafenib), SB590885, XL281 (BMS-908662), and GSK 2118436436. In some embodiments, the tumor is substantially non-responsive to PLX-4032.

The present invention further provides methods of selecting a human subject for treatment with a combination of a Wnt pathway inhibitor and a MAPK pathway inhibitor. In some embodiments, the methods comprise determining if the subject has (a) a tumor or cancer comprising a wild-type B-Raf, or (b) a tumor or cancer that is substantially non-responsive to at least one B-Raf inhibitor, wherein if the subject has (a) and/or (b), the subject is selected for treatment with a combination of a Wnt pathway inhibitor and a MAPK pathway inhibitor.

The sequence of wild-type human B-Raf is known in the art (e.g. Accession No. NP_{—}004324.2), the sequence of wild-type N-Ras is known in the art (e.g., Accession No. NM_{—}002524.4), and the sequence of wild-type K-Ras is known in the art (e.g., Accession No. NP_{—}004976). Methods for determining whether a tumor comprises a Raf or Ras mutation or a wild-type Raf or Ras can be undertaken by assessing the nucleotide sequence encoding the B-Raf, N-Ras, and/or K-Ras protein, by assessing the amino acid sequence of the B-Raf, N-Ras, and/or K-Ras protein, or by assessing the characteristics of a putative B-Raf, N-Ras, and/or K-Ras mutant protein.

Methods for detecting a mutation in a Raf or a Ras nucleotide sequence are known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, NexGen sequencing, primer extension reactions, electrophoresis, oligonucleotide ligation assays, hybridization assays, TaqMan assays, SNP genotyping assays, high resolution melting assays, and microarray analyses. In some embodiments, samples may be evaluated for B-Raf mutations by real-time PCR. In real-time PCR, fluorescent probes specific for the most common mutations are used. When a mutation is present, the probe binds and fluorescence is detected. In some embodiments, samples may be evaluated for N-Ras and/or K-Ras mutations by real-time PCR. In some embodiments, B-Raf mutations may be identified using a direct sequencing method of specific regions in the B-Raf gene. In some embodiments, N-Ras and/or K-Ras mutations may be identified using a direct sequencing method of specific regions in the N-Ras and/or K-Ras gene. Direct sequencing will identify all possible mutations in the region analyzed.

Methods for detecting a mutation in a B-Raf, N-Ras, and/or K-Ras protein are known by those of skill in the art. These methods include, but are not limited to, detection of a B-Raf, N-Ras, and/or K-Ras mutant using a binding agent (e.g., an antibody) specific for the mutant protein, protein electrophoresis and Western blotting, and direct peptide sequencing.

Methods for determining whether a tumor or cancer comprises a B-Raf, N-Ras, and/or K-Ras mutation can use a variety of samples. In some embodiments, the sample is taken from a subject having a tumor or cancer. In some embodiments, the sample is taken from a subject having a cancer or tumor that is substantially non-responsive to at least one B-Raf inhibitor. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is processed to a cell lysate. In some embodiments, the sample is processed to DNA or RNA.

In certain embodiments, the tumor is a tumor selected from the group consisting of colorectal tumor, pancreatic tumor, lung tumor, ovarian tumor, liver tumor, breast tumor, kidney tumor, prostate tumor, gastrointestinal tumor, melanoma, cervical tumor, bladder tumor, glioblastoma, and head and neck tumor. In some embodiments, the tumor is melanoma. In certain embodiments, the tumor is a pancreatic tumor. In certain embodiments, the tumor is a colorectal tumor. In certain embodiments, the tumor is a breast tumor. In certain embodiments, the tumor is a prostate tumor. In certain embodiments, the tumor is a lung tumor.

In some embodiments of any of the methods described herein, the Wnt pathway inhibitor is a Wnt-binding agent. In some embodiments, the Wnt pathway inhibitor is a FZD-binding agent. In some embodiments, the Wnt pathway inhibitor is an antibody. In some embodiments, the Wnt pathway inhibitor is an anti-FZD antibody. In some embodiments, the Wnt pathway inhibitor is the antibody 18R5. In some embodiments, the Wnt pathway inhibitor is a soluble receptor. In some embodiments, the Wnt pathway inhibitor is a FZD-Fc soluble receptor. In some embodiments, the Wnt pathway inhibitor is a FZD8-Fc soluble receptor. In some embodiments, the Wnt pathway inhibitor is FZD8-Fc soluble receptor 54F28. In some embodiments, the Wnt pathway inhibitor consists essentially of a polypeptide of SEQ ID NO:27. In some embodiments, the MAPK pathway inhibitor is a MEK inhibitor. In some embodiments, the MAPK inhibitor is BAY 86-9766.

In certain embodiments of any of the methods described herein, the Wnt pathway inhibitor is an antibody comprising (a) a heavy chain CDR1 comprising GFTFSHYTLS (SEQ ID NO:5), a heavy chain CDR2 comprising VISGDGSYTYYADSVKG (SEQ ID NO:6), and a heavy chain CDR3 comprising NFIKYVFAN (SEQ ID NO:7), and (b) a light chain CDR1 comprising SGDNIGSFYVH (SEQ ID NO:8), a light chain CDR2 comprising DKSNRPSG (SEQ ID NO:9), and a light chain CDR3 comprising QSYANTLSL (SEQ ID NO: 10) and is administered in combination with a MAPK pathway inhibitor.

In certain embodiments of any of the methods described herein, the Wnt pathway inhibitor is an antibody comprising (a) a heavy chain CDR1 comprising GFTFSHYTLS (SEQ ID NO:5), a heavy chain CDR2 comprising VISGDGSYTYYADSVKG (SEQ ID NO:6), and a heavy chain CDR3 comprising NFIKYVFAN (SEQ ID NO:7), and (b) a light chain CDR1 comprising SGDNIGSFYVH (SEQ ID NO:8), a light chain CDR2 comprising DKSNRPSG (SEQ ID NO:9), and a light chain CDR3 comprising QSYANTLSL (SEQ ID NO:10) and is administered in combination with a MEK inhibitor.

In certain embodiments of any of the methods described herein, the Wnt pathway inhibitor is an antibody comprising (a) a heavy chain CDR1 comprising GFTFSHYTLS (SEQ ID NO:5), a heavy chain CDR2 comprising VISGDGSYTYYADSVKG (SEQ ID NO:6), and a heavy chain CDR3 comprising NFIKYVFAN (SEQ ID NO:7), and (b) a light chain CDR1 comprising SGDNIGSFYVH (SEQ ID NO:8), a light chain CDR2 comprising DKSNRPSG (SEQ ID NO:9), and a light chain CDR3 comprising QSYANTLSL (SEQ ID NO:10) and is administered in combination with the MAPK pathway inhibitor BAY 86-9766.

In certain embodiments of any of the methods described herein, the Wnt pathway inhibitor is an antibody comprising a heavy chain variable region comprising SEQ ID NO:3 and a light chain variable region comprising SEQ ID NO:4, administered in combination with a MAPK pathway inhibitor.

In certain embodiments of any of the methods described herein, the Wnt pathway inhibitor is an antibody comprising a heavy chain variable region comprising SEQ ID NO:3 and a light chain variable region comprising SEQ ID NO:4, administered in combination with a MEK inhibitor.

In certain embodiments of any of the methods described herein, the Wnt pathway inhibitor is an antibody comprising a heavy chain variable region comprising SEQ ID NO:3 and a light chain variable region comprising SEQ ID NO:4, administered in combination with the MAPK pathway inhibitor BAY 86-9766.

In certain embodiments of any of the methods described herein, the Wnt pathway inhibitor is a FZD-Fc soluble receptor comprising SEQ ID NO:18, SEQ ID NO:55, or SEQ ID NO:58, administered in combination with a MAPK pathway inhibitor. In some embodiments, the Wnt pathway inhibitor is a FZD-Fc soluble receptor comprising SEQ ID NO:18. In some embodiments, the Wnt pathway inhibitor is a FZD-Fc soluble receptor comprising SEQ ID NO:55. In some embodiments, the Wnt pathway inhibitor is a FZD-Fc soluble receptor comprising SEQ ID NO:58. In some embodiments, the MAPK pathway inhibitor is a MEK inhibitor. In some embodiments, the MEK inhibitor is BAY86-9766.

In certain embodiments of any of the methods described herein, the Wnt pathway inhibitor is a FZD-Fc soluble receptor comprising SEQ ID NO:25, SEQ ID NO:26, or SEQ ID NO:27, administered in combination with a MAPK pathway inhibitor. In some embodiments, the MAPK pathway inhibitor is a MEK inhibitor. In some embodiments, the MEK inhibitor is BAY86-9766. In some embodiments, the Wnt pathway inhibitor is a FZD-Fc soluble receptor comprising SEQ ID NO:25, administered in combination with a MEK inhibitor. In some embodiments, the Wnt pathway inhibitor is a FZD-Fc soluble receptor comprising SEQ ID NO:25, administered in combination with the MEK inhibitor BAY86-9766. In some embodiments, the Wnt pathway inhibitor is a FZD-Fc soluble receptor comprising SEQ ID NO:26, administered in combination with a MEK inhibitor. In some embodiments, the Wnt pathway inhibitor is a FZD-Fc soluble receptor comprising SEQ ID NO:26, administered in combination with the MEK inhibitor BAY86-9766. In some embodiments, the Wnt pathway inhibitor is a FZD-Fc soluble receptor comprising SEQ ID NO:27, administered in combination with a MEK inhibitor. In some embodiments, the Wnt pathway inhibitor is a FZD-Fc soluble receptor comprising SEQ ID NO:27, administered in combination with the MEK inhibitor BAY86-9766.

The invention further provides a method of differentiating tumorigenic cells into non-tumorigenic cells comprising contacting the tumorigenic cells with a combination of a Wnt pathway inhibitor and a MAPK pathway inhibitor (for example, by administering the agents to a subject that has a tumor comprising the tumorigenic cells or that has had such a tumor removed). In certain embodiments, the tumorigenic cells are melanoma cells. In certain embodiments, the tumorigenic cells are lung tumor cells. In certain embodiments, the tumorigenic cells are pancreatic tumor cells. In certain embodiments, the tumorigenic cells are colon tumor cells. In some embodiments, the methods comprise using the Wnt-binding agents, FZD-binding agents, and/or MAPK pathway inhibitors described herein. In certain embodiments, the subject is a human.

The use of Wnt pathway inhibitors in combination with MAPK pathway inhibitors described herein to induce the differentiation of cells, including, but not limited to tumor cells, is also provided. In some embodiments, a method of inducing cells to differentiate comprises contacting the cells with an effective amount of a Wnt pathway inhibitor (e.g., a Wnt-binding agent or a FZD-binding agent) in combination with an effective amount of a MAPK pathway inhibitor. Also provided is a method of inducing cells in a tumor to differentiate comprising administering to a subject a therapeutically effective amount of a Wnt pathway inhibitor in combination with a therapeutically effective amount of a MAPK pathway inhibitor. In some embodiments, the methods comprise using the Wnt-binding agents, FZD-binding agents, and/or MAPK pathway inhibitors described herein. In some embodiments, the tumor is a melanoma. In certain embodiments, the tumor is a lung tumor. In certain embodiments, the tumor is a pancreatic tumor. In certain other embodiments, the tumor is a colon tumor.

The present invention further provides pharmaceutical compositions comprising agents that inhibit the Wnt pathway and/or the MAPK pathway. In some embodiments, the pharmaceutical compositions comprise the Wnt-binding agents and polypeptides described herein. In some embodiments, the pharmaceutical compositions comprise the FZD-binding agents and polypeptides described herein. In some embodiments, the pharmaceutical compositions comprise the MAPK pathway inhibitors described herein. These pharmaceutical compositions find use in inhibiting tumor cell growth and treating cancer in human patients. In some embodiments, the FZD-binding agents described herein in combination with MAPK pathway inhibitors find use in the manufacture of a medicament for the treatment of cancer. In some embodiments, the Wnt-binding agents described herein in combination with MAPK pathway inhibitors find use in the manufacture of a medicament for the treatment of cancer.

Formulations are prepared for storage and use by combining a purified agent or antagonist of the present invention with a pharmaceutically acceptable carrier, excipient, and/or stabilizer as a sterile lyophilized powder, aqueous solution, etc. (Remington: The Science and Practice of Pharmacy. 21st Edition, 2005, University of the Sciences, Philadelphia, Pa.). Suitable carriers, excipients, or stabilizers comprise nontoxic buffers such as phosphate, citrate, and other organic acids; salts such as sodium chloride; antioxidants including ascorbic acid and methionine; preservatives (e.g. octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (such as less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose, or sorbitol; salt-forming counter-ions such as sodium; metal complexes (e.g. Zn-protein complexes); and/or non-ionic surfactants such as TWEEN or polyethylene glycol (PEG).

The therapeutic formulation can be in unit dosage form. Such formulations include tablets, pills, capsules, powders, granules, solutions or suspensions in water or non-aqueous media, or suppositories for oral, parenteral, or rectal administration or for administration by inhalation. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and other diluents (e.g. water) to form a solid preformulation composition containing a homogeneous mixture of a compound of the present invention, or a non-toxic pharmaceutically acceptable salt thereof. The solid preformulation composition is then subdivided into unit dosage forms of the type described above. The tablets, pills, etc., of the novel composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, including a number of polymeric acids and mixtures of polymeric acids with such materials as shellac, cetyl alcohol and cellulose acetate.

Pharmaceutical formulations may include the Wnt pathway inhibitors and/or the MAPK pathway inhibitors of the present invention complexed with liposomes (Epstein et al., 1985, PNAS, 82:3688; Hwang et al., 1980, PNAS, 77:4030; and U.S. Pat. Nos. 4,485,045 and 4,544,545). Liposomes with enhanced circulation time are disclosed in U.S. Pat. No. 5,013,556. Liposomes can be generated by the reverse phase evaporation with a lipid composition comprising phosphatidylcholine, cholesterol, and PEG-derivatized phosphatidylethanolamine (PEG-PE). Liposomes are extruded through filters of defined pore size to yield liposomes with the desired diameter.

The Wnt pathway inhibitors and/or MAPK pathway inhibitors can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 21st Edition, 2005, University of the Sciences, Philadelphia, Pa.

In addition, sustained-release preparations can be prepared. Suitable examples of sustained-release preparations include semi-permeable matrices of solid hydrophobic polymers containing the agent, which matrices are in the form of shaped articles (e.g., films or microcapsules). Examples of sustained-release matrices include polyesters, hydrogels such as poly(2-hydroxyethyl-methacrylate) or poly(vinylalcohol), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The Wnt pathway inhibitors and MAPK pathway inhibitors are administered as appropriate pharmaceutical compositions to a human patient according to known methods. The pharmaceutical compositions can be administered in any number of ways for either local or systemic treatment. Suitable methods of administration include, but are not limited to, intravenous (administration as a bolus or by continuous infusion over a period of time), intraarterial, intramuscular (injection or infusion), intratumoral, intraperitoneal, intracerobrospinal, subcutaneous, intra-articular, intrasynovial, intracranial (e.g., intrathecal or intraventricular), or oral. In additional, administration can be topical, (e.g., transdermal patches, ointments, lotions, creams, gels, drops, suppositories, sprays, liquids and powders) or pulmonary (e.g., by inhalation or insufflation of powders or aerosols, including by nebulizer; intratracheal, intranasal, epidermal and transdermal).

For the treatment of a disease, the appropriate dosage(s) of a Wnt pathway inhibitor in combination with a MAPK pathway inhibitor of the present invention depends on the type of disease to be treated, the severity and course of the disease, the responsiveness of the disease, whether the inhibitors are administered for therapeutic or preventative purposes, previous therapy, the patient's clinical history, and so on, all at the discretion of the treating physician. The Wnt pathway inhibitor can be administered one time or as a series of treatments spread over several days to several months, or until a cure is effected or a diminution of the disease state is achieved (e.g., reduction in tumor size). The MAPK pathway inhibitor can be administered one time or as a series of treatments spread over several days to several months, or until a cure is effected or a diminution of the disease state is achieved (e.g., reduction in tumor size). Optimal dosing schedules for each agent can be calculated from measurements of drug accumulation in the body of the patient and will vary depending on the relative potency of an individual agent. The administering physician can determine optimum dosages, dosing methodologies, and repetition rates.

Combined administration can include co-administration, either in a single pharmaceutical formulation or using separate formulations, or consecutive administration in either order but generally within a time period such that all active agents can exert their biological activities simultaneously.

In certain embodiments, dosage of a Wnt pathway inhibitor is from about 0.01 g to about 100 mg/kg of body weight, from about 0.1 μg to about 100 mg/kg of body weight, from about 1 μg to about 100 mg/kg of body weight, from about 1 mg to about 100 mg/kg of body weight, about 1 mg to about 80 mg/kg of body weight from about 10 mg to about 100 mg/kg of body weight, from about 10 mg to about 75 mg/kg of body weight, or from about 10 mg to about 50 mg/kg of body weight. In certain embodiments, the dosage of the Wnt pathway inhibitor is from about 0.1 mg to about 20 mg/kg of body weight. In certain embodiments, dosage can be given once or more daily, weekly, monthly, or yearly. In certain embodiments, the Wnt pathway inhibitor is given once every week, once every two weeks, once every three weeks, or once every month.

In certain embodiments, dosage of a MAPK pathway inhibitor is from about 25 mg to about 3000 mg, from about 100 mg to about 2500 mg, from about 200 mg to about 2000 mg, from about 400 mg to about 1500 mg, about 500 mg to about 1200 mg, from about 750 mg to about 1000 mg. In certain embodiments, the dosage of the MAPK pathway inhibitor is from about 200 to about 2000 mg/kg. In certain embodiments, dosage can be given once or more daily, weekly, monthly, or yearly. In certain embodiments, the MAPK pathway inhibitor is given twice a day or more, once a day, once every 2 days, once every 3 days, once every 4 days, once every 5 days, once every week, once every two weeks, once every three weeks, or once every month.

In some embodiments, an inhibitor may be administered at an initial higher “loading” dose, followed by one or more lower doses. In some embodiments, the frequency of administration may also change. In some embodiments, a dosing regimen may comprise administering an initial dose, followed by additional doses (or “maintenance” doses) once a week, once every two weeks, once every three weeks, or once every month. For example, a dosing regimen may comprise administering an initial loading dose, followed by a weekly maintenance dose of, for example, one-half of the initial dose. Or a dosing regimen may comprise administering an initial loading dose, followed by maintenance doses of, for example one-half of the initial dose every other week. Or a dosing regimen may comprise administering three initial doses for 3 weeks, followed by maintenance doses of, for example, the same amount every other week.

It will be appreciated that the combination of a Wnt pathway inhibitor and a MAPK pathway inhibitor may be administered in any order or concurrently. In certain embodiments, the Wnt pathway inhibitor and the MAPK pathway inhibitor will be administered substantially simultaneously or concurrently. For example, a subject may be given the Wnt pathway inhibitor while also being given the MAPK pathway inhibitor. In some embodiments, the Wnt pathway inhibitor is administered using a dosing regimen that is different than the dosing regimen used for the MAPK pathway inhibitor.

As is known to those of skill in the art, administration of any therapeutic agent may lead to side effects and/or toxicities. In some cases, the side effects and/or toxicities are so severe as to preclude administration of the particular agent at a therapeutically effective dose. In some cases, drug therapy must be discontinued, and other agents may be tried. However, many agents in the same therapeutic class often display similar side effects and/or toxicities, meaning that the patient either has to stop therapy, or if possible, suffer from the unpleasant side effects associated with the therapeutic agent.

Thus, the present invention provides methods of treating cancer in a subject comprising using an intermittent dosing strategy for administering one or both of the agents, which may reduce side effects and/or toxicities associated with administration of a Wnt pathway inhibitor and/or a MAPK pathway inhibitor. In some embodiments, a method for treating cancer in a human subject comprises administering to the subject a therapeutically effective dose of a Wnt pathway inhibitor in combination with a therapeutically effective dose of a MAPK pathway inhibitor, wherein one or both of the inhibitors are administered according to an intermittent dosing strategy. In some embodiments, the intermittent dosing strategy comprises administering an initial dose of a Wnt pathway inhibitor to the subject, and administering subsequent doses of the Wnt pathway inhibitor about once every 2 weeks. In some embodiments, the intermittent dosing strategy comprises administering an initial dose of a Wnt pathway inhibitor to the subject, and administering subsequent doses of the Wnt pathway inhibitor about once every 3 weeks. In some embodiments, the intermittent dosing strategy comprises administering an initial dose of a Wnt pathway inhibitor to the subject, and administering subsequent doses of the Wnt pathway inhibitor about once every 4 weeks. In some embodiments, the Wnt pathway inhibitor is administered using an intermittent dosing strategy and the MAPK pathway inhibitor is administered daily.

Combination therapy with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the agent(s). Combination therapy may decrease the likelihood that resistant cancer cells will develop. In some embodiments, combination therapy comprises a therapeutic agent that affects (e.g., inhibits or kills) non-tumorigenic cells and a therapeutic agent that affects (e.g., inhibits or kills) tumorigenic CSCs.

In some embodiments, the combination of a Wnt pathway inhibitor and a MAPK pathway inhibitor results in additive or synergetic results. In some embodiments, the combination therapy results in an increase in the therapeutic index of the Wnt pathway inhibitor. In some embodiments, the combination therapy results in an increase in the therapeutic index of the MAPK pathway inhibitor. In some embodiments, the combination therapy results in a decrease in the toxicity and/or side effects of the Wnt pathway inhibitor. In some embodiments, the combination therapy results in a decrease in the toxicity and/or side effects of the MAPK pathway inhibitor.

The treating physician can estimate repetition rates for dosing based on measured residence times and concentrations of the drug in bodily fluids or tissues. The progress of therapy can be monitored by conventional techniques and assays.

In certain embodiments, in addition to administering a Wnt pathway inhibitor in combination with a MAPK pathway inhibitor, treatment methods may further comprise administering additional therapeutic agents prior to, concurrently with, and/or subsequently to administration of the Wnt pathway inhibitor and/or the MAPK pathway inhibitor.

In certain other embodiments, the Wnt pathway inhibitor, the MAPK pathway inhibitor and the additional therapeutic agent(s) will be administered substantially simultaneously or concurrently. For example, a subject may be given the Wnt pathway inhibitor and the MAPK pathway inhibitor while undergoing a course of treatment with the additional therapeutic agent (e.g., chemotherapy). In certain embodiments, the Wnt pathway inhibitor and the MAPK pathway inhibitor will be administered within 1 year of the treatment with the additional therapeutic agent. In certain alternative embodiments, the Wnt pathway inhibitor and the MAPK pathway inhibitor will be administered within 10, 8, 6, 4, or 2 months of any treatment with the additional therapeutic agent. In certain other embodiments, the Wnt pathway inhibitor and the MAPK pathway inhibitor will be administered within 4, 3, 2, or 1 week of any treatment with the additional therapeutic agent. In some embodiments, the Wnt pathway inhibitor and the MAPK pathway inhibitor will be administered within 5, 4, 3, 2, or 1 days of any treatment with the additional therapeutic agent. It will further be appreciated that the agents or treatment may be administered to the subject within a matter of hours or minutes (i.e., substantially simultaneously).

Useful classes of additional therapeutic (e.g., anti-cancer) agents include, for example, antitubulin agents, auristatins, DNA minor groove binders, DNA replication inhibitors, alkylating agents (e.g., platinum complexes such as cis-platin, mono(platinum), bis(platinum) and tri-nuclear platinum complexes and carboplatin), anthracyclines, antibiotics, antifolates, antimetabolites, chemotherapy sensitizers, duocarmycins, etoposides, fluorinated pyrimidines, ionophores, lexitropsins, nitrosureas, platinols, performing compounds, purine antimetabolites, puromycins, radiation sensitizers, steroids, taxanes, topoisomerase inhibitors, vinca alkaloids, or the like. In certain embodiments, the additional therapeutic agent is an antimetabolite, an antimitotic, a topoisomerase inhibitor, or an angiogenesis inhibitor.

Therapeutic agents that may be administered in combination with a Wnt pathway inhibitor and a MAPK pathway inhibitor include chemotherapeutic agents. Thus, in some embodiments, the method or treatment involves the administration of a Wnt pathway inhibitor and MAPK pathway inhibitor of the present invention in combination with a chemotherapeutic agent or cocktail of multiple different chemotherapeutic agents. Treatment with a Wnt pathway inhibitor and MAPK pathway inhibitor can occur prior to, concurrently with, or subsequent to administration of chemotherapies. Chemotherapies contemplated by the invention include chemical substances or drugs which are known in the art and are commercially available, such as gemcitabine, irinotecan, doxorubicin, 5-fluorouracil, cytosine arabinoside (“Ara-C”), cyclophosphamide, thiotepa, busulfan, cytoxin, paclitaxel, methotrexate, cisplatin, melphalan, vinblastine, and carboplatin. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in Chemotherapy Service, 1992, M. C. Perry, Editor, Williams & Wilkins, Baltimore, Md.

Chemotherapeutic agents useful in the instant invention also include, but are not limited to, alkylating agents such as thiotepa and cyclosphosphamide; alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK.; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL) and docetaxel (TAXOTERE), chlorambucil; gemcitabine: 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitor RFS 2000; difluoromethylornithine (DMFO); retinoic acid; esperamicins; capecitabine; and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and antiandrogens such as flutamide, nilutamide, bicalutamide, leuprolide, and goserelin; and pharmaceutically acceptable salts, acids or derivatives of any of the above.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (ABRAXANE), DHA-paclitaxel, or PG-paclitaxel. In certain alternative embodiments, the antimitotic agent comprises a vinca alkaloid, such as vincristine, binblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk1.

In some embodiments, treatment can include administration of one or more cytokines (e.g., lymphokines, interleukins, tumor necrosis factors, and/or growth factors) or can be accompanied by surgical removal of tumor or cancer cells or any other therapy deemed necessary by a treating physician.

In certain embodiments, treatment involves the administration of a Wnt pathway inhibitor and a MAPK pathway inhibitor in combination with radiation therapy. Treatment with the Wnt pathway inhibitor and the MAPK pathway inhibitor can occur prior to, concurrently with, or subsequent to administration of radiation therapy. Any dosing schedules for such radiation therapy can be used as determined by the skilled practitioner.

The present invention further provides methods of screening agents for efficacy in inhibiting Wnt signaling, in inhibiting MAPK signaling, for anti-tumor activity, and/or activity against cancer stem cells. In certain embodiments, the method comprises comparing the level of one or more differentiation markers and/or one or more stemness markers in a first tumor (e.g., a tumor comprising cancer stem cells) that has been exposed to a combination of agents to the level of the one or more differentiation markers in a second tumor that has not been exposed to the agents. In some embodiments, the method comprises: (a) exposing a first tumor, but not a second tumor, to the agents; (b) assessing the level of one or more differentiation markers and/or one or more stemness markers in the first and second tumors; and (c) comparing the level of the one or more differentiation markers in the first tumor and the level of the one or more differentiation markers in the second tumor. In certain embodiments, the (a) increased levels of the one or more differentiation markers in the first tumor relative to the levels of the one or more differentiation markers in the second tumor indicates anti-tumor (or anti-cancer stem cell) activity; and (b) decreased levels of the one or more stemness markers indicate anti-tumor (or anti-cancer stem cell) activity. In certain embodiments, the agents are a Wnt pathway inhibitor in combination with a MAPK pathway inhibitor. In some embodiments, the Wnt pathway inhibitor is an anti-Wnt antibody. In some embodiments, the Wnt pathway inhibitor is an anti-FZD antibody. In certain embodiments, the Wnt pathway inhibitor is a FZD-Fc soluble receptor. In some embodiments, the MAPK pathway inhibitor is a MEK inhibitor. In some embodiments, the differentiation markers are dopachrome tautomerase (DCT), microphthalmia-associated transcription factor (MITF), and/or tyrosinase-related protein 1 (TYRP1).

Additional methods for screening agents include, but are not limited to, methods comprising comparing the levels of one or more differentiation markers in a first tumor that has been exposed to a combination of agents to the levels of the one or more differentiation markers in a second tumor that has not been exposed to the agents. In certain embodiments, the methods include comprising (a) exposing a first tumor, but not a second tumor, to the agents; (b) assessing the levels of one or more differentiation markers in the first and second tumors; and (c) comparing the levels of the one or more differentiation markers in the first tumor to the levels of the one or more differentiation markers in the second tumor. In certain embodiments, the agents are a Wnt pathway inhibitor in combination with a MAPK pathway inhibitor. In some embodiments, the Wnt pathway inhibitor is an anti-Wnt antibody. In some embodiments, the Wnt pathway inhibitor is an anti-FZD antibody. In certain embodiments, the Wnt pathway inhibitor is a FZD-Fc soluble receptor. In certain embodiments, the Wnt pathway inhibitor is an inhibitor of the canonical Wnt signaling pathway. In certain embodiments, the Wnt pathway inhibitor inhibits binding of one or more human Wnt proteins to one or more human FZD receptors. In some embodiments, the differentiation markers are DCT, MITF, and/or TYRP1. In certain embodiments, increased levels of one or more differentiation markers in the first tumor relative to levels of one or more differentiation markers in the second tumor indicates efficacy against solid tumor stem cells (CSCs). In certain alternative embodiments, decreased levels of one or more differentiation markers (i.e., negative markers for differentiation) in the first tumor relative to the levels of one or more differentiation markers in the second tumor indicates efficacy against solid tumor stem cells.

### EXAMPLES

### Example 1

### Characterization of Melanomas Tumors

A collection of xenografts have been established which are derived from patient melanoma tumors. The tumors were expanded by in vivo passage in NOD-SCID mice without any intervening in vitro cell culture. Genomic DNA samples were isolated from primary and passaged tumors using a Genomic DNA Extraction Kit (Bioneer Inc., Alameda, Calif.) following the manufacturers' instructions. The quality of the isolated DNA was checked by visualizing the DNA samples on a 1% agarose gel or a 0.8% E-Gel (Invitrogen Corporation, Carlsbad, Calif.). The DNA was confirmed to be intact by the presence of an approximately 20 kb size band with little or no visible degradation. The purified genomic DNA samples were sent to SeqWright Technologies, (Houston, Tex.) for nucleotide sequence analysis. The B-Raf, N-Ras and K-Ras genes were obtained by amplifying genomic DNA samples with the Repli-G Mini Kit (Qiagen, Valencia, Calif.) followed by PCR amplification and purification. The nucleotide sequences of the B-Raf, N-Ras, and K-Ras genes from each tumor were obtained using an ABI 3730xL DNA Sequencer (Applied Biosystems, Foster City, Calif.).

Of the seven melanoma tumors evaluated, 4 had a wild type B-Raf (OMP-M3, OMP-M4, OMP-OMP-M7, and OMP-M10) and 3 had a mutant B-Raf (OMP-M2, OMP-M5 and OMP-M8) as compared to the human B-Raf sequence (see e.g., Accession No. NP_{—}004324.2). The three melanoma tumors had a mutation in amino acid 600, a valine to glutamate mutation (V600E). The B-Raf valine to glutamate mutation is a known activating mutation. Of the seven melanoma tumors evaluated, 5 had a wild type N-Ras (OMP-M2, OMP-M4, OMP-M5, OMP-M8, and OMP-M10) and 2 had a mutant N-Ras (OMP-M3 and OMP-M7) as compared to the human N-Ras sequence (see e.g., Accession No. NM_{—}002524.4). Both OMP-M3 and OMP-M7 had a mutation in amino acid 61, although OMP-M3 had a glutamine to lysine mutation (Q61K) and OMP-M7 had a glutamine to arginine mutation (Q61R). Mutations at amino acid 61 of the human N-Ras gene are known to be activating mutations. Of the seven melanoma tumors evaluated, 5 had a wild type K-Ras (OMP-M2, OMP-M3, OMP-M4, OMP-M7, and OMP-M8), and 2 had a mutation in K-Ras (OMP-M5 and OMP-M10) as compared to the human K-Ras sequence (see e.g., Accession No. NP_{—}004976). OMP-M10 had a mutation in amino acid 12, a glycine to valine mutation (G12V) that is known to be an activating mutation. OMP-M5 had a leucine to phenylalanine substitution in amino acid 6, which is believed to be a polymorphism and is not known to be an activating mutation in K-Ras.

TABLE 1

| Tumor | B-Raf | N-Ras | K-Ras |
|---|---|---|---|
| OMP-M2 | V600E | WT | WT |
| OMP-M3 | WT | Q61K | WT |
| OMP-M4 | WT | WT | WT |
| OMP-M5 | V600E | WT | L6F |
| OMP-M7 | WT | Q61R | WT |
| OMP-M8 | V600E | WT | WT |
| OMP-M10 | WT | WT | G12V |

### Example 2

### Inhibition of OMP-M3, OMP-M7, and OMP-M10 Tumor Growth In Vivo

Single cell suspensions of OMP-M3, OMP-M7 and OMP-M10 melanoma tumor xenografts (20,000 cells) were injected subcutaneously into 6-8 week old NOD/SCID mice in a 1:1 suspension with Matrigel. Tumors were allowed to grow until they reached an average volume of 200 mm³. The mice were randomized (n=10 per group) and treated with anti-FZD antibody 18R5, control antibody 1B7.11, MEK inhibitor BAY 86-9766, or a combination of antibody 18R5 and BAY 86-9766. Mice were treated once a week with control antibody 1B7.11 or anti-FZD antibody 18R5 at a dose of 20 mg/kg, administered intraperitoneally. Mice were treated with BAY 86-9766 at a dose of 15 mg/kg daily for 5 days each week, administered orally. For combination treatment, mice were administered anti-FZD antibody 18R5 (20 mg/kg) once a week and BAY 86-9766 (15 mg/kg) daily for 5 days each week. Tumor growth was monitored and tumor volumes were measured with electronic calipers at the indicated time points. Data are expressed as mean±S.E.M.

As shown in FIG. 1, single agent treatment with anti-FZD antibody 18R5 at 20 mg/kg once a week had no appreciable effect on OMP-M3, OMP-M7, or OMP-M10 tumor growth as compared to control antibody (FIGS. 1A, 1B and 1C, respectively). Treatment with the MEK inhibitor BAY 86-9766 at 15 mg/kg daily for 5 days each week resulted in reduced tumor growth in both OMP-M3 (FIG. 1A) and OMP-M7 (FIG. 1B) as compared to control antibody. Treatment with BAY 86-9766 resulted in minimal reduced tumor growth in OMP-M10 (FIG. 1C). Surprisingly, the combination of anti-FZD antibody 18R5 and BAY 86-9766 reduced tumor growth to a significantly greater extent than BAY 86-9766 alone, despite the fact that anti-FZD antibody 18R5 had no or minimal effect as a single agent. These results support the hypothesis that targeting more than one signaling pathway will enhance anti-tumor effects and that combination therapy may increase sensitivity of a tumor to an agent that otherwise was not efficacious.

As reported in Example 1, OMP-M3, OMP-M7, and OMP-M10 tumors all contain a wild-type B-Raf gene, but have acquired mutant Ras genes (N-Ras and K-Ras respectively) that may result in increased MAPK signaling. These results show that combination treatment with a Wnt pathway inhibitor and a MEK inhibitor has a strong anti-tumor effect on N-Ras and K-Ras mutant melanoma tumors, and thus may provide a therapy for patients who are not considered for treatment with B-Raf inhibitors.

### Example 3

### Inhibition of OMP-M8 Tumor Growth In Vivo and Tumorigenicity of Treated Tumor Cells

As shown in Example 1, the OMP-M8 melanoma tumor contains the mutation B-Raf^{V600E}. The OMP-M8 tumor was originally obtained from a patient who initially responded to B-Raf inhibitor therapy but who subsequently developed resistance to the B-Raf inhibitor. OMP-M8 melanoma tumor cells (20,000 cells) were injected subcutaneously into 6-8 week old NOD/SCID mice. Tumors were allowed to grow until the average tumor size was approximately 150 mm³. The animals were randomized into four groups (n=10 per group) and treated with B-Raf inhibitor PLX-4720 at doses of 5 mg/kg, 15 mg/kg, and 45 mg/kg, and methyl cellulose vehicle control (1% v/v). PLX-4720 was administered orally for 5 days each week. Tumor growth was measured with electronic calipers on the indicated days after treatment.

As shown in FIG. 2, the OMP-M8 tumor in a xenograft model was resistant to B-Raf inhibitor PLX-4720 at all doses tested, accurately reproducing the resistance acquired in the treated patient. OMP-M8 was also shown to be resistant to sister compound and FDA-approved PLX-4032 (vemurafenib; ZELBORAF).

Next, single cell suspensions of OMP-M8 tumor xenografts (20,000 cells) were injected subcutaneously into 6-8 week old NOD/SCID mice in a 1:1 suspension with Matrigel. Tumors were allowed to grow until they reached an average volume of approximately 150 mm³. The mice were randomized (n=10 per group) and treated with anti-FZD antibody 18R5, control antibody 1B7.11, MEK inhibitor BAY 86-9766, or a combination of anti-FZD antibody 18R5 and BAY 86-9766. Mice were treated once a week with control antibody 1B7.11 or anti-FZD antibody 18R5 at a dose of 20 mg/kg, administered intraperitoneally. Mice were treated with BAY 86-9766 at a dose of 30 mg/kg daily for 5 days each week administered orally. For combination treatment, mice were administered anti-FZD antibody 18R5 (20 mg/kg) once a week and BAY 86-9766 (30 mg/kg) daily for 5 days each week. Tumor growth was monitored and tumor volumes were measured with electronic calipers at the indicated time points.

As shown in FIG. 3A, treatment with the anti-FZD antibody 18R5 had no effect on the growth of the OMP-M8 melanoma tumor in this experiment, while MEK inhibitor BAY 86-9766 significantly reduced growth of the OMP-M8 tumor. The combination of anti-FZD antibody 18R5 and BAY 86-9766 reduced tumor growth beyond the amount observed with BAY 86-9766 alone. These results demonstrated that the B-Raf inhibitor resistant OMP-M8 tumor was sensitive to treatment with an inhibitor directed to a different target in the MAPK pathway. Furthermore, these results demonstrate that a combination of inhibitors targeting the MAPK pathway and the Wnt pathway offers greater efficacy than just targeting the MAPK pathway.

The OMP-M8 tumors described above were processed to yield single cell suspensions. Mouse cells were depleted from the cell mixtures using biotinylated anti-H2K^{d} and anti-CD45 antibodies and streptavidin-conjugated magnetic beads. The remaining human tumor cells were serially transplanted into a new cohort of mice. 10 or 100 tumor cells from each treatment group were injected into the flanks of NOD-SCID mice (n=10 mice per group). Tumors were allowed to grow for 51 days with no treatment and tumor volumes were measured with electronic calipers.

FIG. 3B shows the tumor volume from individual mice in each group. Cells isolated from mice treated with anti-FZD antibody 18R5 had greatly decreased tumorigenicity as compared to cells isolated from mice treated with control antibody. This was surprising, because during the treatment phase anti-FZD antibody 18R5-treated mice did not demonstrate any reduced tumor growth. Cells isolated from mice treated with anti-MEK inhibitor BAY 86-9766 also had significantly decreased tumorigenicity as compared to cells isolated from mice treated with control antibody. Importantly, cells isolated from mice treated with a combination of the anti-FZD antibody 18R5 and BAY 86-9766 demonstrated a significant and striking lack of tumor growth, greater than either agent alone. These results show that inhibiting both the Wnt pathway and the MAPK pathway has a clear and additive effect in reducing tumorigenicity and cancer stem cells.

### Example 4

### Inhibition of OMP-LU33 Lung Tumor Growth In Vivo

Single cell suspensions of OMP-LU33 lung tumor xenografts (10,000 cells) were injected subcutaneously into 6-8 week old NOD/SCID mice in a 1:1 suspension with Matrigel. Tumors were allowed to grow until they reached an average volume of 300 mm³. The mice were randomized (n=9 per group) and treated with anti-FZD antibody 18R5, control antibody 1B7.11, MEK inhibitor BAY 86-9766, or a combination of antibody 18R5 and BAY 86-9766. Mice were treated once a week with control antibody 1B7.11 or anti-FZD antibody 18R5 at a dose of 25 mg/kg, administered intraperitoneally. Mice were treated with BAY 86-9766 at a dose of 50 mg/kg daily for 5 days each week, administered orally. For combination treatment, mice were administered anti-FZD antibody 18R5 (25 mg/kg) once a week and BAY 86-9766 (50 mg/kg) daily for 5 days each week. Tumor growth was monitored and tumor volumes were measured with electronic calipers at the indicated time points. Data are expressed as mean±S.E.M.

As shown in FIG. 4A, single agent treatment with anti-FZD antibody 18R5 at 25 mg/kg once a week had only a minimal effect on OMP-LU33 lung tumor growth as compared to control antibody. Similarly, treatment with the MEK inhibitor BAY 86-9766 at 50 mg/kg daily for 5 days each week resulted in only minimal reduction in tumor growth of OMP-LU33 as compared to control antibody. Surprisingly, the combination of anti-FZD antibody 18R5 and MEK inhibitor BAY 86-9766 reduced tumor growth significantly, despite the fact that anti-FZD antibody 18R5 and BAY 86-9766 both had minimal effect as single agents. These results support the hypothesis that targeting more than one signaling pathway will enhance anti-tumor effects and that combination therapy may increase sensitivity of a tumor to an agent that otherwise was not efficacious. Importantly, these results with a lung tumor xenograft show that the effectiveness of the combination treatment was not limited to only melanoma tumors.

The OMP-LU33 tumor contains a wild-type B-Raf, a wild-type N-Ras, and a mutated K-Ras (G12V). Similar to the results observed in Ras mutant OMP-M3, OMP-M7, and OMP-M10 melanoma tumors, the K-Ras G12V mutant OMP-LU33 tumor growth is significantly inhibited by the combination of anti-FZD antibody 18R5 with MEK inhibitor BAY 86-9766. Therefore these results suggest that co-targeting the Wnt and MAPK pathways in Ras mutant melanoma and lung tumors enhances anti-tumor efficacy.

### Example 5

### Reduction of Active and Total β-Catenin in OMP-M7 and OMP-M10 Melanoma Tumors

Single cell suspensions of OMP-M7 or OMP-M10 melanoma tumor xenografts (20,000 cells) were injected subcutaneously into 6-8 week old NOD/SCID mice in a 1:1 suspension with Matrigel. Tumors were allowed to grow until they reached an average volume of 200 mm³. The mice were randomized and treated with anti-FZD antibody 18R5, control antibody 1B7.11, MEK inhibitor BAY 86-9766, or a combination of antibody 18R5 and BAY 86-9766. Mice were treated once a week with control antibody 1B7.11 or anti-FZD antibody 18R5 at a dose of 20 mg/kg, administered intraperitoneally. Mice were treated with BAY 86-9766 at a dose of 15 mg/kg daily for 5 days each week, administered orally. For combination treatment, mice were administered anti-FZD antibody 18R5 (20 mg/kg) once a week and BAY 86-9766 (15 mg/kg) daily for 5 days each week.

After day 34 for OMP-M7 and day 31 for OMP-M10, tumors were processed into cell lysates (OMP-M7 n=4 and OMP-M10 n=3) using Invitrogen Tissue Extraction Reagent I (Invitrogen/Life Technologies, Grand Island, N.Y.). Approximately 20 ug of total protein was resolved by SDS-PAGE using Invitrogen Novex 4-12% Bis-Tris gels. The proteins were transferred to a nitrocellulose membrane using an Invitrogen iBlot dry blotting system. Western blot analyses were performed using primary antibodies against total β-catenin, active form β-catenin, total ERK, active phosphorylated ERK, and actin. Blots were developed using enhanced chemiluminescence (ECL Plus Western Blotting Detection Reagents, GE Healthcare Life Sciences, Piscataway, N.J.). Western blot results were scanned and quantified by densitometry using NIH ImageJ software.

As shown in FIGS. 5A and SB, OMP-M7 melanoma tumors treated with MEK inhibitor BAY 86-9766 alone showed decreased amounts of total and active β-catenin as well as decreased amounts of phosphorylated ERK. In addition, the combination of MEK inhibitor BAY 86-9766 with anti-FZD antibody 18R5 reduced total β-catenin to a greater extent than the MEK inhibitor alone despite the fact that 18R5 appeared to have no effect as a single agent. As shown in FIGS. 5C and 5D, OMP-M10 melanoma tumors treated with MEK inhibitor BAY 86-9766 alone showed little to no effect on total and active β-catenin, but showed a significant reduction in active phosphorylated ERK. In contrast to OMP-M7, the combination of MEK inhibitor BAY 86-9766 with anti-FZD antibody 18R5 did not reduce active or total β-catenin.

### Example 6

### RNA Analysis of Treated OMP-M3, OMP-M7 and OMP-M10 Tumors

Single cell suspensions of OMP-M3, OMP-M7 or OMP-M10 melanoma tumor xenografts were injected subcutaneously into 6-8 week old NOD/SCID mice in a 1:1 suspension with Matrigel. Tumors were allowed to grow until they reached an average volume of 200 mm. The mice were randomized and treated with anti-FZD antibody 18R5, control antibody 1B7.11, MEK inhibitor BAY 86-9766, or a combination of 18R5 and BAY 86-9766. Mice were treated once a week with control antibody 1B7.11 or anti-FZD antibody 18R5 at a dose of 20 mg/kg, administered intraperitoneally. Mice were treated with BAY 86-9766 at a dose of 15 mg/kg daily for 5 days each week, administered orally. For combination treatment, mice were administered anti-FZD antibody 18R5 (20 mg/kg) once a week and BAY 86-9766 (15 mg/kg) daily for 5 days each week. After approximately 4 weeks, total RNA from OMP-M3, OMP-M7, and OMP-M10 tumors was isolated using a RNeasy Isolation Kit (Qiagen, Valencia, Calif.). RNA was quantified and 25 ng was used for Taqman gene expression analyses (Life Technologies, Grand Island, N.Y.). In addition, OMP-M3, OMP-M7 and OMP-M10 formalin-fixed, paraffin-embedded tumor sections from each treatment group were analyzed by immunohistochemistry (IHC) using antibodies against E-cadherin (Cell Signaling Technology, Danvers, Mass.). After antibody staining, slides were scanned using an Aperio ScanScope instrument (Aperio, Vista, Calif.) and the human cell populations were analyzed for positive staining using Aperio software.

As shown in FIG. 6A, OMP-M3, OMP-M7, and OMP-M10 tumors all demonstrated increased expression of melanocyte lineage genes, DCT, MITF and TYRP1 after treatment with the combination of anti-FZD antibody 18R5 and MEK inhibitor BAY 86-9766. These results suggest that combined inhibition of the Wnt and MAPK pathways may enhance differentiation of melanoma cells into a less tumorigenic state.

IHC analysis showed that treatment with the combination of anti-FZD antibody 18R5 and MEK inhibitor BAY 86-9766 significantly increased the percentage of cells staining positive for E-cadherin over treatment with either 18R5 or BAY 86-9766 alone (FIG. 6B). In many cancer types, loss of E-cadherin expression coincides with acquisition of an invasive phenotype and development of metastatic disease. In normal melanocytes. E-cadherin mediates melanocyte-keratinocyte interactions and loss of E-cadherin expression or a change in its cellular distribution is associated with early phases of melanoma. Thus, these results suggest that combined inhibition of the Wnt and MAPK pathways may increase E-cadherin expression and decrease the invasiveness of melanoma cells.

### Example 7

### Inhibition of OMP-LU56 Lung Tumor Growth In Vivo

Single cell suspensions of OMP-LU56 lung tumor xenografts (50,000 cells) were injected subcutaneously into 6-8 week old NOD/SCID mice in a 1:1 suspension with Matrigel. Tumors were allowed to grow until they reached an average volume of 150 mm³. The mice were randomized (n=9 per group) and treated with anti-FZD antibody 18R5, control antibody 1B7.11, MEK inhibitor BAY 86-9766, or a combination of antibody 18R5 and BAY 86-9766. Mice were treated every two weeks (Q2W) with control antibody 1B7.11 or anti-FZD antibody 18R5 at a dose of 25 mg/kg, administered intraperitoneally. Mice were treated with BAY 86-9766 at a dose of 30 mg/kg daily for 5 days each week, administered orally. For combination treatment, mice were administered anti-FZD antibody 18R5 (25 mg/kg) once a week and BAY 86-9766 (30 mg/kg) daily for 5 days each week. Tumor growth was monitored and tumor volumes were measured with electronic calipers at the indicated time points. Data are expressed as mean±S.E.M.

As shown in FIG. 4B, single agent treatment with anti-FZD antibody 18R5 at 25 mg/kg once a week had no detectable effect on OMP-LU56 lung tumor growth as compared to control antibody. Treatment with the MEK inhibitor BAY 86-9766 at 30 mg/kg daily for 5 days each week resulted in significant reduction (56%) in tumor growth of OMP-LU56 as compared to control antibody. Surprisingly, the combination of anti-FZD antibody 18R5 and MEK inhibitor BAY 86-9766 reduced tumor growth to a greater extent than BAY 86-9766 alone (78%), despite the fact that anti-FZD antibody 18R5 had no effect as a single agent. As seen with OMP-LU33 (see Example 4), these results support the hypothesis that targeting more than one signaling pathway will enhance anti-tumor effects and that combination therapy may increase sensitivity of a tumor to an agent that otherwise is not efficacious.

The OMP-LU56 tumor contains a wild-type B-Raf, a wild-type N-Ras, and a mutated K-Ras (G12C). Similar to the results observed in Ras mutant OMP-M3, OMP-M7, and OMP-M10 melanoma tumors and OMP-LU33 lung tumor, the K-Ras G12C mutant OMP-LU56 tumor growth is significantly inhibited by the combination of anti-FZD antibody 18R5 with MEK inhibitor BAY 86-9766. Therefore these results further support the suggestion that co-targeting the Wnt and MAPK pathways in Ras mutant melanoma and lung tumors enhances anti-tumor efficacy.

It is understood that the examples and embodiments described herein are for illustrative purposes only and that various modifications or changes in light thereof will be suggested to person skilled in the art and are to be included within the spirit and purview of this application.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

SEQUENCES

SEQ ID NO: 1 18R5 Heavy chain amino acid sequence with predicted
signal sequence underlined
MKHLWFFLLLVAAPRWVLSEVQLVESGGGLVQPGGSLRLSCAASGFTFSHYTLSWVRQAP
GKGLEWVSVISGDGSYTYYADSVKGRFTISSDNSKNTLYLQMNSLRAEDTAVYYCARNFI
KYVFANWGQGTLVTVSSASTKGPSVFPLAPCSRSTSESTAALGCLVKDYFPEPVTVSWNS
GALTSGVHTFPAVLQSSGLYSLSSVVTVPSSNFGTQTYTCNVDHKPSNTKVDKTVERKCC
VECPPCPAPPVAGPSVFLFPPKPKDTLMISRTPEVTCVVVDVSHEDPEVQFNWYVDGVEV
HNAKTKPREEQFNSTFRVVSVLTVVHQDWLNGKEYKCKVSNKGLPAPIEKTISKTKGQPR
EPQVYTLPPSREEMTKNQVSLTCLVKGFYPSDIAVEWESNGQPENNYKTTPPMLDSDGSF
FLYSKLTVDKSRWQQGNVFSCSVMHEALHNHYTQKSLSLSPGK
SEQ ID NO: 2 18R5 Light chain amino acid sequence with predicted
signal sequence underlined
MAWALLLLTLLTQGTGSWADIELTQPPSVSVAPGQTARISCSGDNIGSFYVHWYQQKPGQ
APVLVIYDKSNRPSGIPERFSGSNSGNTATLTISGTQAEDEADYYCQSYANTLSLVFGGG
TKLTVLGQPKAAPSVTLFPPSSEELQANKATLVCLISDFYPGAVTVAWKADSSPVKAGVE
TTTPSKQSNNKYAASSYLSLTPEQWKSHRSYSCQVTHEGSTVEKTVAPTECS
SEQ ID NO: 3 18R5 Heavy chain variable region amino acid sequence
EVQLVESGGGLVQPGGSLRLSCAASGFTFSHYTLSWVRQAPGKGLEWVSVISGDGSYTYY
ADSVKGRFTISSDNSKNTLYLQMNSLRAEDTAVYYCARNFIKYVFANWGQGTLVTVSS
SEQ ID NO: 4 18R5 Light chain variable region amino acid sequence
DIELTQPPSVSVAPGQTARISCSGDNIGSFYVHWYQQKPGQAPVLVIYDKSNRPSGIPER
FSGSNSGNTATLTISGTQAEDEADYYCQSYANTLSLVFGGGTKLTVLG
SEQ ID NO: 5 18R5 Heavy chain CDR1
GFTFSHYTLS
SEQ ID NO: 6 18R5 Heavy chain CDR2
VISGDGSYTYYADSVKG
SEQ ID NO: 7 18R5 Heavy chain CDR3
NFIKYVFAN
SEQ ID NO: 8 18R5 Light chain CDR1
SGDNIGSFYVH
SEQ ID NO: 9 18R5 Light chain CDR2
DKSNRPSG
SEQ ID NO: 10 18R5 Light chain CDR3
QSYANTLSL
SEQ ID NO: 11 Human FZD1 Fri domain amino acid sequence without
predicted signal sequence
QQPPPPPQQQQSGQQYNGERGISVPDHGYCQPISIPLCTDIAYNQTIMPNLLGHTNQEDA
GLEVHQFYPLVKVQCSAELKFFLCSMYAPVCTVLEQALPPCRSLCERARQGCEALMNKFG
FQWPDTLKCEKFPVHGAGELCVGQNTSDKGT
SEQ ID NO: 12 Human FZD2 Fri domain amino acid sequence without
predicted signal sequence
QFHGEKGISIPDHGFCQPISIPLCTDIAYNQTIMPNLLGHTNQEDAGLEVHQFYPLVKVQ
CSPELRFFLCSMYAPVCTVLEQAIPPCRSICERARQGCEALMNKFGFQWPERLRCEHFPR
HGAEQICVGQNHSEDG
SEQ ID NO: 13 Human FZD3 Fri domain amino acid sequence without
predicted signal sequence
HSLFSCEPITLRMCQDLPYNTTFMPNLLNHYDQQTAALAMEPFHPMVNLDCSRDF
RPFLCALYAPICMEYGRVTLPCRRLCQRAYSECSKLMEMFGVPWPEDMECSRFPDCDEPY
PRLVDL
SEQ ID NO: 14 Human FZD4 Fri domain amino acid sequence without
predicted signal sequence
FGDEEERRCDPIRISMCQNLGYNVTKMPNLVGHELQTDAELQLTTFTPLIQYGCSSQLQF
FLCSVYVPMCTEKINIPIGPCGGMCLSVKRRCEPVLKEFGFAWPESLNCSKFPPQNDHNH
MCMEGPGDEEV
SEQ ID NO: 15 Human FZD5 Fri domain amino acid sequence without
predicted signal sequence
ASKAPVCQEITVPMCRGIGYNLTHMPNQFNHDTQDEAGLEVHQFWPLVEIQCSPDLRFFL
CSMYTPICLPDYHKPLPPCRSVCERAKAGCSPLMRQYGFAWPERMSCDRLPVLGRDAEVL
CMDYNRSEATT
SEQ ID NO: 16 Human FZD6 Fri domain amino acid sequence without
predicted signal sequence
HSLFTCEPITVPRCMKMAYNMTFFPNLMGHYDQSIAAVEMEHFLPLANLECSPNIETFLC
KAFVPTCIEQIHVVPPCRKLCEKVYSDCKKLIDTFGIRWPEELECDRLQYCDETVPVTFD
PHTEFLG
SEQ ID NO: 17 Human FZD7 Fri domain amino acid sequence without
predicted signal sequence
QPYHGEKGISVPDHGFCQPISIPLCTDIAYNQTILPNLLGHTNQEDAGLEVHQFYPLVKV
QCSPELRFFLCSMYAPVCTVLDQAIPPCRSLCERARQGCEALMNKFGFQWPERLRCENFP
VHGAGEICVGQNTSDGSG
SEQ ID NO: 18 Human FZD8 Fri domain amino acid sequence without
predicted signal sequence
ASAKELACQEITVPLCKGIGYNYTYMPNQFNHDTQDEAGLEVHQFWPLVEIQCSPDLKFF
LCSMYTPICLEDYKKPLPPCRSVCERAKAGCAPLMRQYGFAWPDRMRCDRLPEQGNPDTL
CMDYNRTDLTT
SEQ ID NO: 19 Human FZD9 Fri domain amino acid sequence without
predicted signal sequence
LEIGRFDPERGRGAAPCQAVEIPMCRGIGYNLTRMPNLLGHTSQGEAAAELAEFAPLVQY
GCHSHLRFFLCSLYAPMCTDQVSTPIPACRPMCEQARLRCAPIMEQFNFGWPDSLDCARL
PTRNDPHALCMEAPENA
SEQ ID NO: 20 Human FZD10 Fri domain amino acid sequence without
predicted signal sequence
ISSMDMERPGDGKCQPIEIPMCKDIGYNMTRMPNLMGHENQREAAIQLHEFAPLVEYGCH
GHLRFFLCSLYAPMCTEQVSTPIPACRVMCEQARLKCSPIMEQFNFKWPDSLDCRKLPNK
NDPNYLCMEAPNNG
SEQ ID NO: 21 Human IgG₁ Fc region
DKTHTCPPCPAPELLGGPSVFLFPPKPKDTLMI$RTPEVTCVVVDVSHEDPEVKFNWYVD
GVEVHNAKTKPREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO: 22 Human IgG₁ Fce region
KSSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
SEQ ID NO: 23 Human IgG₁ Fc region
EPKSSDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSPGK
SEQ ID NO: 24 Human IgG₂ Fc region
CVECPPCPAPPVAGPSVFLFPPKPKDTLMISRTPEVTCVVVDVSHEDPEVQFNWYVDGVE
VHNAKTKPREEQFNSTFRVVSVLTVVHQDWLNGKEYKCKVSNKGLPAPIEKTISKTKGQP
REPQVYTLPPSREEMTKNQVSLTCLVKGFYPSDIAVEWESNGQPENNYKTTPPMLDSDGS
FFLYSKLTVDKSRWQQGNVFSCSVMHEALHNHYTQKSLSLSPGK
SEQ ID NO: 25 FZD8-Fc variant 54F03 amino acid sequence (without
predicted signal sequence)
ASAKELACQEITVPLCKGIGYNYTYMPNQFNHDTQDEAGLEVHQFWPLVEIQCSPDLKFF
LCSMYTPICLEDYKKPLPPCRSVCERAKAGCAPLMRQYGFAWPDRMRCDRLPEQGNPDTL
CMDYNRTDLTTGRADKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
K
SEQ ID NO: 26 FZD8-Fc variant 54F16, 54F17, 54F18, 54F23, 54F25, 54F27,
54F29, 54F31, and 54F34 amino acid sequence (without predicted signal
sequence)
ASAKELACQEITVPLCKGIGYNYTYMPNQFNHDTQDEAGLEVHQFWPLVEIQCSPDLKFF
LCSMYTPICLEDYKKPLPPCRSVCERAKAGCAPLMRQYGFAWPDRMRCDRLPEQGNPDTL
CMDYNRTDLTTKSSDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
K
SEQ ID NO: 27 FZD8-Fc variant 54F19, 54F20, 54F24, 54F26, 54F28, 54F30,
54F32, 54F34 and 54F35 amino acid sequence (without predicted signal
sequence)
ASAKELACQEITVPLCKGIGYNYTYMPNQFNHDTQDEAGLEVHQFWPLVEIQCSPDLKFF
LCSMYTPICLEDYKKPLPPCRSVCERAKAGCAPLMRQYGFAWPDRMRCDRLPEQGNPDTL
CMDYNRTDLTTEPKSSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLS
PGK
SEQ ID NO: 28 FZD8-Fc variant 54F03 amino acid sequence with signal
sequence
MEWGYLLEVTSLLAALALLQRSSGAAAASAKELACQEITVPLCKGIGYNYTYMPNQFNHD
TQDEAGLEVHQFWPLVEIQCSPDLKFFLCSMYTPICLEDYKKPLPPCRSVCERAKAGCAP
LMRQYGFAWPDRMRCDRLPEQGNPDTLCMDYNRTDLTTGRADKTHTCPPCPAPELLGGPS
VFLFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLYSKLTVDKSRWQQ
GNVFSCSVMHEALHNHYTQKSLSLSPGK
SEQ ID NO: 29 FZDa-Fc variant 54F16 amino acid sequence with signal
sequence
MEWGYLLEVTSLLAALALLQRSSGAAAASAKELACQEITVPLCKGIGYNYTYMPNQFNHD
TQDEAGLEVHQFWPLVEIQCSPDLKFFLCSMYTPICLEDYKKPLPPCRSVCERAKAGCAP
LMRQYGFAWPDRMRCDRLPEQGNPDTLCMDYNRTDLTTKSSDKTHTCPPCPAPELLGGPS
VFLFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLYSKLTVDKSRWQQ
GNVFSCSVMHEALHNHYTQKSLSLSPGK
SEQ ID NO: 30 FZD8-Fc variant 54E26 with signal sequence
MEWGYLLEVTSLLAALFLLQRSPIVHAASAKELACQEITVPLCKGIGYNYTYMPNQFNHD
TQDEAGLEVHQFWPLVEIQCSPDLKFFLCSMYTPICLEDYKKPLPPCRSVCERAKAGCAP
LMRQYGFAWPDRMRCDRLPEQGNPDTLCMDYNRTDLTTEPKSSDKTHTCPPCPAPELLGG
PSVFLFPPKPKDTLMISRTPEVTCVVVDVSHEDPEVKRNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPGK
SEQ ID NO: 31 FZD8-Fc variant 54F28 with signal sequence
MEWGYLLEVTSLLAALLLLQRSPFVHAASAKELACQEITVPLCKGIGYNYTYMPNQFNHD
TQDEAGLEVHQFWPLVEIQCSPDLKFFLCSMYTPICLEDYKKPLPPCRSVCERAKAGCAP
LMRQYGFAWPDRMRCDRLPEQGNPDTLCMDYNRTDLTTEPKSSDKTHTCPPCPAPELLGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPGK
SEQ ID NO: 32 Human Wnt1 C-terminal cysteine rich domain (aa 288-370)
DLVYFEKSPNFCTYSGRLGTAGTAGRACNSSSPALDGCELLCCGRGHRTRTQRVTERCNC
TFHWCCHVSCRNCTHTRVLHECL
SEQ ID NO: 33 Human Wnt2 C-temainal cysteine rich domain (aa 267-360)
DLVYFENSPDYCIRDREAGSLGTAGRVCNLTSRGMDSCEVMCCGRGYDTSHVTRMTKCGC
KFHWCCAVRCQDCLEALDVHTCKAPKNADWTTAT
SEQ ID NO: 34 Human Wnt2b C-terminal cysteine rich domain (aa 298-391)
DLVYFDNSPDYCVLDKAAGSLGTAGRVCSKTSKGTDGCEIMCCGRGYDTTRVTRVTQCEC
KFHWCCAVRCKECRNTVDVHTCKAPKKAEWLDQT
SEQ ID NO: 35 Human Wnt3 C-terminal cysteine rich domain (aa 273-355)
DLVYYENSPNFCEPNPETGSFGTRDRTCNVTSHGIDGCDLLCCGRGHNTRTEKRKEKCHC
IFHWCCYVSCQECIRIYDVHTCK
SEQ ID NO: 36 Human Wnt3a C-terminal cysteine rich domain (aa 270-352)
DLVYYEASPNFCEPNPETGSFGTRDRTCNVSSHGIDGCDLLCCGRGHNARAERRREKCRC
VFHWCCYVSCQECTRVYDVHTCK
SEQ ID NO: 37 Human Wnt7a C-terminal cysteine rich domain (aa 267-359)
DLVYIEKSPNYCEEDPVTGSVGTQGRACNKTAPQASGCDLMCCGRGYNTHQYARVWQCNC
KFHWCCYVKCNTCSERTEMYTCK
SEQ ID NO: 38 Human Wnt7b C-terminal cysteine rich domain (aa 267-349)
DLVYIEKSPNYCEEDAATGSVGTQGRLCNRTSPGADGCDTMCCGRGYNTHQYTKVWQCNC
KFHWCCFVKCNTCSERTEVFTCK
SEQ ID NO: 39 Human Wnt8a C-terminal cysteine rich domain (aa 248-355)
ELIFLEESPDYCTCNSSLGIYGTEGRECLQNSHNTSRWERRSCGRLCTECGLQVEERKTE
VISSCNCKFQWCCTVKCDQCRHVVSKYYCARSPGSAQSLGRVWFGVYI
SEQ ID NO: 40 Human Wnt8b C-terminal cysteine rich domain (aa 245-351)
ELVHLEDSPDYCLENKTLGLLGTEGRECLRRGRALGRWELRSCRRLCGDCGLAVEERRAE
TVSSCNCKFHWCCAVRCEQCRRRVTKYFCSRAERPRGGAAHKPGRKP
SEQ ID NO: 41 Human Wntl Oa C-terminal cysteine rich domain (aa 335-417)
DLVYFEKSPDFCEREPRLDSAGTVGRLCNKSSAGSDGCGSMCCGRGHNILRQTRSERCHC
RFHWCCFVVCEECRITEWVSVCK
SEQ ID NO: 42 Human Wntl Ob C-terminal cysteine rich domain (aa 307-389)
ELVYFEKSPDFCERDPTMGSPGTRGRACNKTSRLLDGCGSLCCGRGHNVLRQTRVERCHC
RFHWCCYVLCDECKVTEWVNVCK
SEQ ID NO: 43 Linker
ESGGGGVT
SEQ ID NO: 44 Linker
LESGGGGVT
SEQ ID NO: 45 Linker
GRAQVT
SEQ ID NO: 46 Linker
WRAQVT
SEQ ID NO: 47 Linker
ARGRAQVT
SEQ ID NO: 48 Human FZD1 amino acids 116-227
CQPISIPLCTDIAYNQTIMPNLLGHTNQEDAGLEVHQFYPLVKVQCSAELKFFLCSMYAP
VCTVLEQALPPCRSLCERARQGCEALMNKFGFQWPDTLKCEKEPVHGAGELC
SEQ ID NO: 49 Human FZD2 amino acids 39-150
CQPISIPLCTDIAYNQTIMPNLLGHTNQEDAGLEVHQFYPLVKVQCSPELRFFLCSMYAP
VCTVLEQAIPPCRSICERARQGCEALMNKFGFQWPERLRCEHFPRHGAEQIC
SEQ ID NO: 50 Human FZD3 amino acids 28-133
CEPITLRMCQDLPYNTTFMPNLLNHYDQQTAALAMEPFHPMVNLDCSRDFRPFLCALYAP
ICMEYGRVTLPCRRLCQRAYSECSKLMEMEGVPWPEDMECSRFPDC
SEQ ID NO: 51 Human FZD4 amino acids 48-161
CDPIRISMCQNLGYNVTKMPNLVGHELQTDAELQLTTFTPLIQYGCSSQLQFFLCSVYVP
MCTEKINIPIGPCGGMCLSVKRRCEPVLKEFGFAWPESLNCSKFPPQNDHNHMC
SEQ ID NO: 52 Human FZD5 amino acids 33-147
CQEITVPMCRGIGYNLTHMPNQFNHDTQDEAGLEVHQFWPLVEIQCSPDLRFFLCSMYTP
ICLPDYHKPLPPCRSVCERAKAGCSPLMRQYGFAWPERMSCDRLPVLGRDAEVLC
SEQ ID NO: 53 Human FZD6 amino acids 24-129
CEPITVPRCMKMAYNMTFFPNLMGHYDQSIAAVEMEHFLPLANLECSPNIETFLCKAFVP
TCIEQIHVVPPCRKLCEKVYSDCKKLIDTFGIRWPEELECDRLQYC
SEQ ID NO: 54 Human FZD7 amino acids 49-160
CQPISIPLCTDIAYNQTILPNLLGHTNQEDAGLEVHQFYPLVKVQCSPELRFFLCSMYAP
VCTVLDQAIPPCRSLCERARQGCEALMNKFGFQWPERLRCENFPVHGAGEIC
SEQ ID NO: 55 Human FZD8 amino acids 35-148
CQEITVPLCKGIGYNYTYMPNQFNHDTQDEAGLEVHQFWPLVEIQCSPDLKFFLCSMYTP
ICLEDYKKPLPPCRSVCERAKAGCAPLMRQYGFAWPDRMRCDRLPEQGNPDTLC
SEQ ID NO: 56 Human FZD9 amino acids 39-152
CQAVEIPMCRGIGYNLTRMPNLLGHTSQGEAAAELAEFAPLVQYGCHSHLRFFLCSLYAP
MCTDQVSTPIPACRPMCEQARLRCAPIMEQFNFGWPDSLDCARLPTRNDPHALC
SEQ ID NO: 57 Human FZD10 amino acids 34-147
CQPIEIPMCKDIGYNMTRMPNLMGHENQREAAIQLHEFAPLVEYGCHGHLRFFLCSLYAP
MCTEQVSTPIPACRVMCEQARLKCSPIMEQFNFKWPDSLDCRKLPNKNDPNYLC
SEQ ID NO: 58 Human FZD8 Fri domain amino acid sequence without
predicted signal sequence
ASAKELACQEITVPLCKGIGYNYTYMPNQFNHDTQDEAGLEVHQFWPLVEIQCSPDLKFF
LCSMYTPICLEDYKKPLPPCRSVCERAKAGCAPLMRQYGFAWPDRMRCDRLPEQGNPDTL
CMDYNRTDL
SEQ ID NO: 59 Human IgG1 Fc region
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
SEQ ID NO: 60 18R5 Heavy chain amino acid sequence without predicted
signal sequence
EVQLVESGGGLVQPGGSLRLSCAASGFTFSHYTLSWVRQAPGKGLEWVSVISGDGSYTYY
ADSVKGRFTISSDNSKNTLYLQMNSLRAEDTAVYYCARNFIKYVFANWGQGTLVTVSSAS
TKGPSVFPLAPCSRSTSESTAALGCLVKDYFPEPVTVSWNSGALTSGVHTFPAVLQSSGL
YSLSSVVTVPSSNFGTQTYTCNVDHKPSNTKVDKTVERKCCVECPPCPAPPVAGPSVFLF
PPKPKDTLMISRTPEVTCVVVDVSHEDPEVQFNWYVDGVEVHNAKTKPREEQFNSTFRVV
SVLTVVHQDWLNGKEYKCKVSNKGLPAPIEKTISKTKGQPREPQVYTLPPSREEMTKNQV
SLTCLVKGFYPSDIAVEWESNGQPENNYKTTPPMLDSDGSFFLYSKLTVDKSRWQQGNVF
SCSVMHEALHNHYTQKSLSLSPGK
SEQ ID NO: 61 18R5 Light chain amino acid sequence without predicted
signal sequence
DIELTQPPSVSVAPGQTARISCSGDNIGSFYVHWYQQKPGQAPVLVIYDKSNRPSGIPER
FSGSNSGNTATLTISGTQAEDEADYYCQSYANTLSLVFGGGTKLTVLGQPKAAPSVTLFP
PSSEELQANKATLVCLISDFYPGAVTVAWKADSSPVKAGVETTTPSKQSNNKYAASSYLS
LTPEQWKSHRSYSCQVTHEGSTVEKTVAPTECS

1-64. (canceled)

65. A method of treating cancer in a subject comprising: administering to the subject a therapeutically effective amount of a Wnt pathway inhibitor in combination with a therapeutically effective amount of a mitogen-activated protein kinase (MAPK) pathway inhibitor.

66. The method of claim 65, wherein the Wnt pathway inhibitor is an antibody or a soluble receptor.

67. The method of claim 65, wherein the Wnt pathway inhibitor is an antibody that specifically binds at least one FZD protein or portion thereof and the antibody comprises:
a) a heavy chain CDR1 comprising GFTFSHYTLS (SEQ ID NO:5), a heavy chain CDR2 comprising VISGDGSYTYYADSVKG (SEQ ID NO:6), and a heavy chain CDR3 comprising NFIKYVFAN (SEQ ID NO:7), and
b) a light chain CDR1 comprising SGDNIGSFYVH (SEQ ID NO:8), a light chain CDR2 comprising DKSNRPSG (SEQ ID NO:9), and a light chain CDR3 comprising QSYANTLSL (SEQ ID NO:10).

68. The method of claim 65, wherein the Wnt pathway inhibitor is an antibody that specifically binds at least one FZD protein or portion thereof and the antibody comprises:
a) a heavy chain variable region having at least 90% sequence identity to SEQ ID NO:3; and
b) a light chain variable region having at least 90% sequence identity to SEQ ID NO:4.

69. The method of claim 65, wherein the Wnt pathway inhibitor is antibody OMP-18R5.

70. The method of claim 67, wherein the antibody is a monoclonal antibody, a recombinant antibody, a chimeric antibody, a humanized antibody, a human antibody, a antibody fragment comprising an antigen-binding site, a bispecific antibody, an IgG1 antibody, or an IgG2 antibody.

71. The method of claim 65, wherein the Wnt pathway inhibitor is soluble receptor that specifically binds at least one Wnt protein or portion thereof and the soluble receptor comprises the Fri domain of human FZD8.

72. The method of claim 71, wherein the Fri domain of human FZD8 consists essentially of SEQ ID NO:18 or SEQ ID NO:58.

73. The method of claim 71, wherein the soluble receptor comprises a human Fc region.

74. The method of claim 66, wherein the soluble receptor comprises SEQ ID NO:27.

75. The method of claim 65, wherein the Wnt pathway inhibitor is soluble receptor 54F28.

76. The method of claim 65, wherein the MAPK pathway inhibitor is selected from a group consisting of: a MEK inhibitor, a Ras inhibitor, a Raf inhibitor, and a ERK inhibitor.

77. The method of claim 76, wherein the MAPK pathway inhibitor is a MEK inhibitor selected from the group consisting of: BAY 86-9766 (RDEA119), PD0325901, CI-1040, PD98059, PD318088, GSK 120212 (JTP-74057), AZD8330 (ARRY-424704), AZD6244 (ARRY-142886), ARRY-162, ARRY-300, AS703026, U0126, CH4987655, and TAK-733.

78. The method of claim 76, wherein the MAPK pathway inhibitor is a Raf inhibitor selected from the group consisting of: GDC-0879, PLX-4720, PLX-4032 (vemurafenib), RAF265, BAY 73-4506, BAY 43-9006 (sorafenib), SB590885, XL281 (BMS-908662), and GSK 2118436436.

79. The method of claim 65, which further comprises administering an additional therapeutic agent.

80. The method of claim 65, wherein the cancer/tumor:
(a) comprises a N-Ras mutation or a K-Ras mutation;
(b) comprises a B-Raf mutation;
(c) comprises a wild-type B-Raf; and/or
(d) is substantially nonresponsive to at least one B-Raf inhibitor.

81. A method of treating a human subject who has a tumor or cancer which is substantially non-responsive to at least one B-Raf inhibitor, comprising administering to the subject a therapeutically effective amount of a Wnt pathway inhibitor in combination with a therapeutically effective amount of a MAPK pathway inhibitor.

82. A method of inhibiting growth of a melanoma tumor in a subject, comprising administering to the subject a therapeutically effective amount of an anti-FZD antibody or a FZD-Fc soluble receptor in combination with a MEK inhibitor.

83. The method of claim 82, wherein the anti-FZD antibody is 18R5 and the MEK inhibitor is BAY 86-9766.

84. The method of claim 82, wherein the FZD-Fc soluble receptor is 54F28 and the MEK inhibitor is BAY 86-9766.


SEQUENCE LISTING (61 sequences)

SEQ ID NO: 1 - protein, 463 aa, Artificial sequence
  18R5 Heavy chain
MKHLWFFLLLVAAPRWVLSEVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 2 - protein, 232 aa, Artificial sequence
  18R5 Light chain
MAWALLLLTLLTQGTGSWADIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 118 aa, Artificial sequence
  18R5 Heavy chain variable region
EVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSS

SEQ ID NO: 4 - protein, 108 aa, Artificial sequence
  18R5 Light chain variable region
DIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLG

SEQ ID NO: 5 - protein, 10 aa, Artificial sequence
  18R5 Heavy chain CDR1
GFTFSHYTLS

SEQ ID NO: 6 - protein, 17 aa, Artificial sequence
  18R5 Heavy chain CDR2
VISGDGSYTYYADSVKG

SEQ ID NO: 7 - protein, 9 aa, Artificial sequence
  18R5 Heavy chain CDR3
NFIKYVFAN

SEQ ID NO: 8 - protein, 11 aa, Artificial sequence
  18R5 Light chain CDR1
SGDNIGSFYVH

SEQ ID NO: 9 - protein, 8 aa, Artificial sequence
  18R5 Light chain CDR2
DKSNRPSG

SEQ ID NO: 10 - protein, 9 aa, Artificial sequence
  18R5 Light chain CDR3
QSYANTLSL

SEQ ID NO: 11 - protein, 151 aa, Homo sapiens
QQPPPPPQQQQSGQQYNGERGISVPDHGYCQPISIPLCTDIAYNQTIMPNLLGHTNQEDAGLEVHQFYPLVKVQCSAELKFFLCSMYAPVCTVLEQALPPCRSLCERARQGCEALMNKFGFQWPDTLKCEKFPVHGAGELCVGQNTSDKGT

SEQ ID NO: 12 - protein, 136 aa, Homo sapiens
QFHGEKGISIPDHGFCQPISIPLCTDIAYNQTIMPNLLGHTNQEDAGLEVHQFYPLVKVQCSPELRFFLCSMYAPVCTVLEQAIPPCRSICERARQGCEALMNKFGFQWPERLRCEHFPRHGAEQICVGQNHSEDG

SEQ ID NO: 13 - protein, 121 aa, Homo sapiens
HSLFSCEPITLRMCQDLPYNTTFMPNLLNHYDQQTAALAMEPFHPMVNLDCSRDFRPFLCALYAPICMEYGRVTLPCRRLCQRAYSECSKLMEMFGVPWPEDMECSRFPDCDEPYPRLVDL

SEQ ID NO: 14 - protein, 131 aa, Homo sapiens
FGDEEERRCDPIRISMCQNLGYNVTKMPNLVGHELQTDAELQLTTFTPLIQYGCSSQLQFFLCSVYVPMCTEKINIPIGPCGGMCLSVKRRCEPVLKEFGFAWPESLNCSKFPPQNDHNHMCMEGPGDEEV

SEQ ID NO: 15 - protein, 131 aa, Homo sapiens
ASKAPVCQEITVPMCRGIGYNLTHMPNQFNHDTQDEAGLEVHQFWPLVEIQCSPDLRFFLCSMYTPICLPDYHKPLPPCRSVCERAKAGCSPLMRQYGFAWPERMSCDRLPVLGRDAEVLCMDYNRSEATT

SEQ ID NO: 16 - protein, 127 aa, Homo sapiens
HSLFTCEPITVPRCMKMAYNMTFFPNLMGHYDQSIAAVEMEHFLPLANLECSPNIETFLCKAFVPTCIEQIHVVPPCRKLCEKVYSDCKKLIDTFGIRWPEELECDRLQYCDETVPVTFDPHTEFLG

SEQ ID NO: 17 - protein, 138 aa, Homo sapiens
QPYHGEKGISVPDHGFCQPISIPLCTDIAYNQTILPNLLGHTNQEDAGLEVHQFYPLVKVQCSPELRFFLCSMYAPVCTVLDQAIPPCRSLCERARQGCEALMNKFGFQWPERLRCENFPVHGAGEICVGQNTSDGSG

SEQ ID NO: 18 - protein, 131 aa, Homo sapiens
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTT

SEQ ID NO: 19 - protein, 137 aa, Homo sapiens
LEIGRFDPERGRGAAPCQAVEIPMCRGIGYNLTRMPNLLGHTSQGEAAAELAEFAPLVQYGCHSHLRFFLCSLYAPMCTDQVSTPIPACRPMCEQARLRCAPIMEQFNFGWPDSLDCARLPTRNDPHALCMEAPENA

SEQ ID NO: 20 - protein, 134 aa, Homo sapiens
ISSMDMERPGDGKCQPIEIPMCKDIGYNMTRMPNLMGHENQREAAIQLHEFAPLVEYGCHGHLRFFLCSLYAPMCTEQVSTPIPACRVMCEQARLKCSPIMEQFNFKWPDSLDCRKLPNKNDPNYLCMEAPNNG

SEQ ID NO: 21 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 230 aa, Homo sapiens
KSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 232 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 224 aa, Homo sapiens
CVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 25 - protein, 361 aa, Artificial sequence
  FZD8-Fc variant 54F03
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTGRADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 361 aa, Artificial sequence
  FZD8-Fc variant 54F16, 54F17, 54F18, 54F23, 54F25, 54F27, 54F29, 54F31, and 54F34
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 363 aa, Artificial sequence
  FZD8-Fc variant 54F19, 54F20, 54F24, 54F26, 54F28, 54F30, 54F32, 54F34 and 54F35
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 388 aa, Artificial sequence
  FZD8-Fc variant 54F03
MEWGYLLEVTSLLAALALLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTGRADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 388 aa, Artificial sequence
  FZD8-Fc variant 54F16
MEWGYLLEVTSLLAALALLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 390 aa, Artificial sequence
  FZD8-Fc variant 54F26
MEWGYLLEVTSLLAALFLLQRSPIVHAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 390 aa, Artificial sequence
  FZD8-Fc variant 54F28
MEWGYLLEVTSLLAALLLLQRSPFVHAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 83 aa, Homo sapiens
DLVYFEKSPNFCTYSGRLGTAGTAGRACNSSSPALDGCELLCCGRGHRTRTQRVTERCNCTFHWCCHVSCRNCTHTRVLHECL

SEQ ID NO: 33 - protein, 94 aa, Homo sapiens
DLVYFENSPDYCIRDREAGSLGTAGRVCNLTSRGMDSCEVMCCGRGYDTSHVTRMTKCGCKFHWCCAVRCQDCLEALDVHTCKAPKNADWTTAT

SEQ ID NO: 34 - protein, 94 aa, Homo sapiens
DLVYFDNSPDYCVLDKAAGSLGTAGRVCSKTSKGTDGCEIMCCGRGYDTTRVTRVTQCECKFHWCCAVRCKECRNTVDVHTCKAPKKAEWLDQT

SEQ ID NO: 35 - protein, 83 aa, Homo sapiens
DLVYYENSPNFCEPNPETGSFGTRDRTCNVTSHGIDGCDLLCCGRGHNTRTEKRKEKCHCIFHWCCYVSCQECIRIYDVHTCK

SEQ ID NO: 36 - protein, 83 aa, Homo sapiens
DLVYYEASPNFCEPNPETGSFGTRDRTCNVSSHGIDGCDLLCCGRGHNARAERRREKCRCVFHWCCYVSCQECTRVYDVHTCK

SEQ ID NO: 37 - protein, 83 aa, Homo sapiens
DLVYIEKSPNYCEEDPVTGSVGTQGRACNKTAPQASGCDLMCCGRGYNTHQYARVWQCNCKFHWCCYVKCNTCSERTEMYTCK

SEQ ID NO: 38 - protein, 83 aa, Homo sapiens
DLVYIEKSPNYCEEDAATGSVGTQGRLCNRTSPGADGCDTMCCGRGYNTHQYTKVWQCNCKFHWCCFVKCNTCSERTEVFTCK

SEQ ID NO: 39 - protein, 108 aa, Homo sapiens
ELIFLEESPDYCTCNSSLGIYGTEGRECLQNSHNTSRWERRSCGRLCTECGLQVEERKTEVISSCNCKFQWCCTVKCDQCRHVVSKYYCARSPGSAQSLGRVWFGVYI

SEQ ID NO: 40 - protein, 107 aa, Homo sapiens
ELVHLEDSPDYCLENKTLGLLGTEGRECLRRGRALGRWELRSCRRLCGDCGLAVEERRAETVSSCNCKFHWCCAVRCEQCRRRVTKYFCSRAERPRGGAAHKPGRKP

SEQ ID NO: 41 - protein, 83 aa, Homo sapiens
DLVYFEKSPDFCEREPRLDSAGTVGRLCNKSSAGSDGCGSMCCGRGHNILRQTRSERCHCRFHWCCFVVCEECRITEWVSVCK

SEQ ID NO: 42 - protein, 83 aa, Homo sapiens
ELVYFEKSPDFCERDPTMGSPGTRGRACNKTSRLLDGCGSLCCGRGHNVLRQTRVERCHCRFHWCCYVLCDECKVTEWVNVCK

SEQ ID NO: 43 - protein, 8 aa, Artificial sequence
  Linker
ESGGGGVT

SEQ ID NO: 44 - protein, 9 aa, Artificial sequence
  Linker
LESGGGGVT

SEQ ID NO: 45 - protein, 6 aa, Artificial sequence
  Linker
GRAQVT

SEQ ID NO: 46 - protein, 6 aa, Artificial sequence
  Linker
WRAQVT

SEQ ID NO: 47 - protein, 8 aa, Artificial sequence
  Linker
ARGRAQVT

SEQ ID NO: 48 - protein, 112 aa, Homo sapiens
CQPISIPLCTDIAYNQTIMPNLLGHTNQEDAGLEVHQFYPLVKVQCSAELKFFLCSMYAPVCTVLEQALPPCRSLCERARQGCEALMNKFGFQWPDTLKCEKFPVHGAGELC

SEQ ID NO: 49 - protein, 112 aa, Homo sapiens
CQPISIPLCTDIAYNQTIMPNLLGHTNQEDAGLEVHQFYPLVKVQCSPELRFFLCSMYAPVCTVLEQAIPPCRSICERARQGCEALMNKFGFQWPERLRCEHFPRHGAEQIC

SEQ ID NO: 50 - protein, 106 aa, Homo sapiens
CEPITLRMCQDLPYNTTFMPNLLNHYDQQTAALAMEPFHPMVNLDCSRDFRPFLCALYAPICMEYGRVTLPCRRLCQRAYSECSKLMEMFGVPWPEDMECSRFPDC

SEQ ID NO: 51 - protein, 114 aa, Homo sapiens
CDPIRISMCQNLGYNVTKMPNLVGHELQTDAELQLTTFTPLIQYGCSSQLQFFLCSVYVPMCTEKINIPIGPCGGMCLSVKRRCEPVLKEFGFAWPESLNCSKFPPQNDHNHMC

SEQ ID NO: 52 - protein, 115 aa, Homo sapiens
CQEITVPMCRGIGYNLTHMPNQFNHDTQDEAGLEVHQFWPLVEIQCSPDLRFFLCSMYTPICLPDYHKPLPPCRSVCERAKAGCSPLMRQYGFAWPERMSCDRLPVLGRDAEVLC

SEQ ID NO: 53 - protein, 106 aa, Homo sapiens
CEPITVPRCMKMAYNMTFFPNLMGHYDQSIAAVEMEHFLPLANLECSPNIETFLCKAFVPTCIEQIHVVPPCRKLCEKVYSDCKKLIDTFGIRWPEELECDRLQYC

SEQ ID NO: 54 - protein, 112 aa, Homo sapiens
CQPISIPLCTDIAYNQTILPNLLGHTNQEDAGLEVHQFYPLVKVQCSPELRFFLCSMYAPVCTVLDQAIPPCRSLCERARQGCEALMNKFGFQWPERLRCENFPVHGAGEIC

SEQ ID NO: 55 - protein, 114 aa, Homo sapiens
CQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLC

SEQ ID NO: 56 - protein, 114 aa, Homo sapiens
CQAVEIPMCRGIGYNLTRMPNLLGHTSQGEAAAELAEFAPLVQYGCHSHLRFFLCSLYAPMCTDQVSTPIPACRPMCEQARLRCAPIMEQFNFGWPDSLDCARLPTRNDPHALC

SEQ ID NO: 57 - protein, 114 aa, Homo sapiens
CQPIEIPMCKDIGYNMTRMPNLMGHENQREAAIQLHEFAPLVEYGCHGHLRFFLCSLYAPMCTEQVSTPIPACRVMCEQARLKCSPIMEQFNFKWPDSLDCRKLPNKNDPNYLC

SEQ ID NO: 58 - protein, 129 aa, Homo sapiens
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDL

SEQ ID NO: 59 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 444 aa, Artificial sequence
  18R5 Heavy chain
EVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 61 - protein, 213 aa, Artificial sequence
  18R5 Light chain
DIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS